(19) United States
(12) Patent Application Publication
(10) Pub. No.: US 2021/0038589 A1
(43) Pub. Date: Feb. 11, 2021

(54) USES, COMPOSITIONS AND METHODS

(71) Applicants: Andre Fisahn, Stockholm (SE); Mikael Altun, Stockholm (SE)

(72) Inventors: Andre Fisahn, Stockholm (SE); Mikael Altun, Stockholm (SE)

(21) Appl. No.: 16/979,756

(22) PCT Filed: Mar. 15, 2019
(86) PCT No.: PCT/EP2019/056559

§ 371 Date: Sep. 10, 2020

(30) Foreign Application Priority Data
Mar. 15, 2018 (GB) 1804163.2

(51) Int. Cl. A61K 31/454; A61P 25/28; A61K 31/451; A61K 49/00

(52) U.S. Cl. CPC A61K 31/454; A61K 49/0008; A61K 31/451; A61P 25/28

(57) ABSTRACT

The present invention relates generally to calcium channel inhibitors for use in treating and/or preventing an amyloid disease of the nervous system. The invention also relates to related pharmaceutical compositions, kits and screening methods.

The present invention relates generally to calcium channel inhibitors for use in treating and/or preventing an amyloid disease of the nervous system. The invention also relates to related pharmaceutical compositions, kits and screening methods.

Amyloid diseases of the nervous system place an increasing social and economic burden on society. Several such diseases such as Alzheimer's disease (AD), Parkinson's disease (PD), Huntington's disease (HD), amyotrophic lateral sclerosis (ALS) and prion diseases manifest the hallmark of protein misfolding and aggregation, and formation of inclusion bodies.

The most prevalent of these diseases is the neurodegenerative disease, Alzheimer's disease, which affects about 10 percent of the adult population over sixty-five years old in North America. Parkinson's disease and Huntington's disease have similar amyloid origins. These diseases can be sporadic (occurring without any family history) or familial (inherited). Regardless of the type, the risk of getting any of these diseases increases dramatically with age. One mechanistic explanation for this correlation is that, as individuals age (or as a result of mutations), the delicate balance of the synthesis, folding, and degradation of proteins is perturbed, resulting in the production and accumulation of misfolded proteins that form aggregates (Reynaud, E. (2010) Nature Education 3(9):28).

The amyloid-β hypothesis of Alzheimer's disease points toward the accumulation of amyloid-β peptide (Aβ) as one of the main culprits for the physiological changes seen during progression of AD. These include the desynchronization of action potentials, the consequent development of aberrant brain rhythms relevant for cognition (gamma oscillations—a functional biomarker for cognitive decline), and the final emergence of cognitive deficits in patients. Similar mechanisms and patient outcomes are observed in other amyloid diseases such as PD (which is characterised by the aggregation-prone protein α-synuclein), and HD (which is characterised by the aggregation-prone protein huntingtin).

Today, no disease modifying treatment for AD, PD or HD exists, and symptomatic therapies are being used with limited effects.

Treatment strategies have so far focused on decreasing the amount of aggregating protein, inhibition of peptide misfolding, and elimination of the toxic forms of peptides by immunotherapies.

In the healthy brain, protein degradation mechanisms involve either the ubiquitin-proteasome system (UPS) or the lysosomal system (ALP). The UPS is a highly regulated mechanism of intracellular protein degradation and turnover. Through the concerted actions of a series of enzymes, proteins are marked for proteasomal degradation by being linked to the polypeptide co-factor, ubiquitin. Work over the last 30 years has established the importance of regulating protein ubiquitylation in a wide range of cellular functions including cell cycle control, transcriptional regulation, and diverse aspects of cell signalling. These processes are disturbed in many human diseases such as cancer, immunological disorders and neurodegeneration involving abnormal accumulations of proteins in neurons. Impairment of the UPS by mutations has been linked to late-onset neurodegenerative diseases such as AD, PD and polyglutamine diseases (polyQ's), as well as neurodegeneration in the context of normal aging.

However, the results of clinical trials focused on such approaches have so far been disappointing. Immunotherapies have failed at clinical trial stage because the employed antibodies are too large to cross cell membranes in beneficial numbers and therefore fail to reach their intended target.

Thus, new approaches for treating and/or preventing amyloid diseases of the nervous system and neurodegenerative diseases are needed.

Against this background, the present inventors have surprisingly discovered a class of compounds for use in treating and/or preventing an amyloid disease of the nervous system. That class of compounds modulates cellular calcium concentration by inhibiting the activity of calcium channels—specifically, voltage-gated T-type calcium channels—which the inventors have found is effective in reducing protein aggregation and/or protein misfolding, and therefore has utility in treating and/or preventing an amyloid disease of the nervous system. Some members of that class of compounds are FDA-approved drugs, which have been proposed and/or used in the treatment of disorders that are unrelated to amyloid diseases.

In a first aspect, the invention provides a calcium channel inhibitor for use in treating and/or preventing an amyloid disease of the nervous system in an individual.

In a second aspect, the invention provides use of a calcium channel inhibitor in the manufacture of a medicament for treating and/or preventing an amyloid disease of the nervous system in an individual.

In a third aspect, the invention provides a method for treating and/or preventing an amyloid disease of the nervous system in an individual comprising administering a calcium channel inhibitor to an individual.

As discussed further below and in the accompanying Examples, without being bound by theory the inventors believe that the calcium channel inhibitors of the invention can increase proteolysis through the basal mechanism of modulating cellular calcium concentration by inhibiting the voltage-gated T-type calcium channels.

By “calcium channel” we include the meaning of a plasma membrane protein containing calcium-selective pores that are opened by depolarisation of the membrane voltage. Such channels may produce depolarisation-induced calcium entry in neurons, muscle and other excitable cells, as well as some non-excitable cells. Functions mediated by calcium channels include contraction of muscle, release of neurotransmitters and hormones by neurons and neuroendocrine cells, and control of gene transcription. Calcium channels are multi-subunit proteins encoded by many separate genes, and the resulting proteins often govern distinct functional roles within a given cell type. They are targets for modulation by many intracellular signalling pathways including G proteins and phosphorylation. Calcium channels play pivotal roles in many human diseases, particularly of the cardiac and nervous systems, including pain, seizure, hypertension and migraine (Bean, B. P. and McDonough, S. I. 2010. Calcium Channels. eLS).

By the term “inhibitor” we include a natural or synthetic agent that blocks or reduces the activity of a target protein (for example, a calcium channel). An inhibitor may also be termed an “antagonist”, and may act with competitive, uncompetitive, or non-competitive inhibition. An inhibitor can bind reversibly or irreversibly to the target protein.

Thus, by “calcium channel inhibitor” we include the meaning of any synthetic or natural agent, that directly and/or indirectly reduces (wholly or partially) or prevents calcium ion flux through the calcium channel by a measurable level. Such inhibitors may modify one or more site on or near the pore of a calcium channel, or cause a conformational change on the calcium channel.

It will be appreciated that a calcium channel inhibitor that directly inhibits and/or prevents calcium ion flux through the calcium channel may bind to, and act directly on, the calcium channel. It will also be appreciated that a calcium channel inhibitor that indirectly inhibits and/or prevents calcium ion flux through the calcium channel may modulate the activity of another protein that itself directly inhibits the calcium channel, and so effects the calcium channel through a pathway of cellular events.

The calcium channel inhibitor may decrease the level of calcium flux through the channel by at least 10%, or 20%, or 30%, or 40%, or 50%, or 60%, or 70%, or 80%, or 90%, or 95%, or 99%, or more.

Thus, in one embodiment, the calcium channel inhibitor may be a calcium channel blocker (which inhibits calcium channel flux through the channel by 100%). By “calcium channel blocker” we include the meaning of any synthetic or natural agent, that directly and/or indirectly blocks calcium ion flux through the calcium channel.

Methods for measuring calcium flux are known in the art and include the use of calcium ion-selective electrodes, or calcium imaging using a calcium indicator. Calcium indicators are fluorescent molecules that can respond to the binding of Ca2⁺ ions by changing their fluorescence properties, and such indicators include chemical indicators or genetically encoded calcium indicators (GECI) (Busche M. A. (2018) In Vivo Two-Photon Calcium Imaging of Hippocampal Neurons in Alzheimer Mouse Models. In: Perneczky R. (eds) Biomarkers for Alzheimer's Disease Drug Development. Methods in Molecular Biology, vol 1750. Humana Press, New York, N.Y. Vőfély, Berecz T, SzabóE, Szebényi K, Hathy E, Orbán T I, Sarkadi B, Homolya L, Marchetto M C, Réthelyi J M, Apáti Á. Mol Cell Neurosci. 2018 Feb. 6; 88:222-230 and Kopljar I, Hermans A N, Teisman A, Gallacher D J, Lu HR. J Pharmacol Toxicol Methods. 2018 Feb. 6; 91:80-86).

By “amyloid disease” we include a pathological condition characterised by the presence of amyloid fibrils. The term “amyloid” is intended as a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra.

Local deposition of amyloid is common in the brain, particularly in elderly individuals. The most frequent type of amyloid in the brain is composed primarily of Aβ peptide fibrils, resulting in dementia associated with sporadic (non-hereditary) Alzheimer's disease. In fact, the incidence of sporadic Alzheimer's disease greatly exceeds forms shown to be hereditary. Nevertheless, fibril peptides forming plaques are very similar in both types.

By “amyloid disease” we also include a neurodegenerative disease and/or condition, such as those characterised by the presence of amyloid fibrils. The terms “amyloid disease” and “neurodegenerative disease and/or condition” may be used interchangeably herein. Neurodegenerative diseases are a heterogeneous group of disorders that are characterized by the progressive degeneration of the structure and function of the central nervous system or peripheral nervous system, and/or characterised by an impairment or absence of a normal neurological function, or presence of an abnormal neurological function in an individual, or group of individuals. As used herein, neurodegenerative disease also includes neurodegeneration which causes a morphological and/or functional abnormality of a neural cell or a population of neural cells.

Diseases and/or conditions of the invention may be chronic or acute. Examples of such diseases comprise or consist of: Alexander disease, Alper's disease, Alzheimer's Disease (AD), Amyotrophic lateral sclerosis (ALS), Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease (CJD), Frontotemporal Dementia, Huntington's disease (HD), HIV associated dementia, Kennedy's disease, Krabbe disease, Lewy body dementia, Machado-Joseph disease (Spinocerebellar ataxia type 3), Multiple sclerosis (MS), Multiple System Atrophy, Neuroborreliosis, Parkinson disease (PD), Pelizaeus-Merzbacher Disease, Pick's disease, Primary lateral sclerosis, Polyglutamine Disease, Prion diseases, Refsum's disease, Sandhoff disease, Schilder's disease, Sub-Acute Combined Degeneration of the Cord Secondary to Pernicious Anaemia, Schizophrenia, Spinocerebellar ataxia (multiple types with varying characteristics), Spinal muscular atrophy, Steele-Richardson-Olszewski disease, Tabes dorsalis, Transmissible Spongiform Encephalopathy, and Charcot-Marie-Tooth disease.

It will be appreciated that the disease and/or condition may be caused and/or increased by a brain injury (such as stroke or an injury caused by a brain surgery) or may occur as part of the aging process (which is associated with a significant reduction in the number of neural stem cells).

Amyloid disease diagnosis may be based on an individual's history, physical examination, and cognitive testing, using methods known in the art. Cognitive testing may include neuropsychological tests of global cognitive function, memory, language, visuospatial ability, processing speed and attention/working memory/executive function. Physical examination may include positron emission tomography (PET) which provides regional and pathophysiological information non-invasively in living human subjects. Using PET tau and amyloid β ligands can be visualised. Structural imaging such as Computed Tomography (CT) or Magnetic Resonance Imaging (MRI) which produces a picture of the brain that allows identification of such features as volume loss or abnormal structural features such as white matter disease, masses, or vascular abnormalities, which are considered indications of neuronal degeneration, may also be used. Cerebrospinal Fluid (CSF) tests are also possible, in which the relationship between cerebrospinal fluid in which markers of disease in the CSF (for example, in the case of Alzheimer's Disease, amyloid beta (Aβ) and tau proteins) are quantified. Genetic testing is also available. Symptomatic treatments are available. Prevention may include a healthy diet and keeping the brain active.

It will be appreciated that the amyloid disease of the nervous system described herein may also be termed a proteopathy. By “proteopathy” we include diseases caused by misfolded proteins and characterised by the presence of aberrant protein aggregates in the cell. Accordingly, the calcium channel inhibitor of the first, second and third aspects of the invention, may have utility in the treatment and/or prevention of proteopathies, such as proteopathies of the nervous system.

By “amyloid disease of the nervous system”, we include diseases, such as those defined above, which occur in the nervous system—the network of nerves and cells that carry messages to and from the brain and spinal cord to various parts of the body. The nervous system includes both the Central nervous system (“CNS”) and Peripheral nervous system (“PNS”). The central nervous system is made up of the brain, spinal cord and nerves. The peripheral nervous system consists of sensory neurons, ganglia and nerves that connect to one another and to the central nervous system.

By “treating” or “treatment” we include administering therapy to reverse, reduce, alleviate, arrest or cure the symptoms, clinical signs, and/or underlying pathology of a disorder, disease, injury or condition, in a manner to improve or stabilize an individual's condition. Thus, in the context of the present invention, “treatment” refers to administration of the calcium channel inhibitor of the invention to an individual in need thereof, with the expectation that they will obtain a therapeutic benefit. For example, treating an amyloid disease of the nervous system and/or a neurodegenerative disease and/or condition, includes the full or partial restoration of cognitive function and/or rhythmic electrical activity (such as in the gamma-frequency range) which underlies higher brain processes such as learning, memory and cognition.

The meaning of the term “preventing” or “prevention” will be appreciated to those skilled in the art, and when used in relation to a condition (such as a neurodegenerative disease or any other medical condition), it includes administration of a therapy which reduces the frequency of, or delays the onset of, symptoms, clinical signs, and/or underlying pathology of a specific disorder, disease, injury or medical condition in an individual relative to an individual who does not receive that therapy. The term “prophylactic” treatment may be used interchangeably with “preventing” and “prevention”. “Prophylactic treatment” includes administration of a therapy prior to clinical manifestation of the condition (e.g., neurogenerative disease or other unwanted state of the individual) (i.e., it protects the individual against developing the unwanted condition) conversely, if the therapy is administered after manifestation of the unwanted condition, the treatment is therapeutic, (i.e., it is intended to diminish, ameliorate, or stabilize the existing unwanted condition or side effects thereof).

By “an individual” we include the meaning of a subject in need of treatment and/or prevention of a disease or condition as described herein. The individual may be a vertebrate, such as a vertebrate mammal, for example a human, or a non-human mammal, such as a domestic animal (for example, cat, dog, rabbit, cow, sheep, pig, mouse or other rodent).

Preferably, the amyloid disease of the nervous system is characterised by protein aggregation and/or protein misfolding.

By “protein” we include an amino-acid based polymer (i.e. two or more amino acids joined to each other by peptide bonds or modified peptide bonds, i.e., peptide isosteres), such as a peptide or polypeptide. The terms “protein”, “peptide” and “polypeptide” may be used interchangeably herein. Polypeptides may contain amino acids other than the 20 gene-encoded amino acids and/or amino acid sequences modified either by natural processes (such as post-translational processing), or by chemical modification, as is known in the art.

By “protein aggregation” we include the process by which misfolded proteins adopt a conformation that cause its polymerization into aggregates and organized fibrils. For example, the aggregation of β-amyloid (termed “Aβ”) (as seen in Alzheimer's disease), α-synuclein (as seen in synucleinopathies, such as Parkinson's disease), tau protein (as seen in Alzheimer's), and prion protein (for prion diseases, such as CJD).

By “protein misfolding” we include a situation in which normal protein folding is disrupted. This may be either a deviation from the native folded state or the induced folding of a disordered protein into a pathogenic conformation, such as an amyloid deposit. As a result, misfolded proteins can display “sticky” surfaces and aggregate through several stages eventually assembling into fibrils, such as amyloid fibrils, and such non-functional protein aggregates can be toxic. Misfolding increases the tendency of the protein to self-assemble into stable, structured aggregates, and frequently renders it resistant to normal cellular clearance mechanisms.

Preferably, protein aggregation and/or protein misfolding causes the formation of one or more amyloid deposits.

By “amyloid deposit” we include amyloid bodies and/or aggregates and/or assemblies. The terms amyloid “deposits”, “bodies”, “aggregates” “fibrils” and “assemblies” may be used interchangeably herein. Amyloid deposits are proteinaceous extracellular aggregates associated with a diverse range of disease states, and are composed predominantly of amyloid fibrils (the unbranched, β-sheet rich structures that result from the misfolding and subsequent aggregation of the protein and/or polypeptide). Amyloid deposits may consist of aggregation-protein proteins such as, but not limited to, Aβ peptide, Tau, α-synuclein, Huntingtin, superoxide dismutase (SOD1), FUS, TDP-43, and Prion Protein. Accordingly, it will be appreciated that an amyloid deposit as defined herein may be a neurofibrillary tangle comprising Tau, or an inclusion body comprising a synuclein protein.

In an embodiment, the amyloid deposits are formed in cells of the nervous system. For example, in the brain, spinal cord, nerves, sensory neurons and ganglia.

Accordingly, the calcium channel inhibitor according to the invention may inhibit and/or reduce formation of protein aggregates and misfolded proteins, which are characteristic of neurodegenerative disease.

Preferably, the amyloid disease of the nervous system is selected from the group comprising: Alzheimer's disease (AD), Parkinson's disease (PD), Huntington's disease (HD), amyotrophic lateral sclerosis (ALS), Lewy-body dementia, Frontotemporal Dementia, spongiform encephalopathies (such as Creutzfeldt-Jakob's disease (CJD)) and polyglutamine diseases (polyQ's).

Amyloid diseases and/or neurodegenerative diseases to which the invention is related include, without limitation: Alexander disease, Alper's disease, Alzheimer's Disease (AD), Amyotrophic lateral sclerosis (ALS), Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease (CJD), Frontotemporal Dementia, Huntington's disease (HD), HIV-associated dementia, Kennedy's disease, Krabbe disease, Lewy body dementia, Machado-Joseph disease (Spinocerebellar ataxia type 3), Multiple sclerosis (MS), Multiple System Atrophy, Neuroborreliosis, Parkinson disease (PD), Pelizaeus-Merzbacher Disease, Pick's disease, Primary lateral sclerosis, Polyglutamine Disease, Prion diseases, Refsum's disease, Sandhoff disease, Schilder's disease, Sub-Acute Combined Degeneration of the Cord Secondary to Pernicious Anaemia, Schizophrenia, Spinocerebellar ataxia (multiple types with varying characteristics), Spinal muscular atrophy, Steele-Richardson-Olszewski disease, Tabes dorsalis, Transmissible Spongiform Encephalopathy, and Charcot-Marie-Tooth disease.

Particularly preferred diseases of the invention include (with their associated proteins involved in aggregation): Alzheimer's Disease (which involves Aβ peptide and/or Tau), Parkinson's Disease (which involves α-synuclein), Huntington's Disease (which involves Huntingtin), Amyotrophic Lateral Sclerosis (which involves SOD, FUS, and/or TDP-43), Lewy-body dementia (which involves α-synuclein), Frontotemporal Dementia (which involves Tau), Transmissible Spongiform Encephalopathies such as Creutzfeldt-Jakob's disease (which involves Prion Protein) and polyglutamine diseases (which involves polyQ's).

“Alzheimer's disease”, also referred to as “AD”, is a syndrome with several monogenetic autosomal dominant causes, each related to metabolism of the amyloid precursor protein (APP) and its cleavage to form amyloid β (Aβ) peptides. However, the much more common form(s) of AD does not have a single genetic cause. This last group, referred to as sporadic AD, likely derives from the processes of aging, inherited susceptibilities such as the Epsilon-4 allele of the apolipoprotein (apo) E gene (APOE4), and environmental factors (BMJ 2009; 338:b158). A dominant hypothesis is that all forms of AD share in common increased production or accumulation of Aβ peptides, especially Aβ42.

Amyloid-β peptide (Aβ) is a 39-43 amino acid peptide derived by proteolysis from a large protein known as Beta Amyloid Precursor Protein (“βAPP”). Mutations in βAPP result in familial forms of Alzheimer's disease, Down's syndrome, cerebral amyloid angiopathy, and senile dementia, characterized by cerebral deposition of plaques composed of Aβ fibrils and other components. Known mutations in APP associated with Alzheimer's disease occur proximate to the cleavage sites of β or γ-secretase, or within Aβ. The familial form of Alzheimer's disease represents only 10% of the subject population. Most occurrences of Alzheimer's disease are sporadic cases where APP and Aβ do not possess any mutation.

It will be appreciated by those skilled in the art that AD diagnosis may be based on an individual's history, physical examination, and cognitive testing. Cognitive testing may include neuropsychological tests of global cognitive function, memory, language, visuospatial ability, processing speed and attention/working memory/executive function. Physical examination may include positron emission tomography (PET) which provides regional and pathophysiological information non-invasively in living human subjects. Structural imaging such as Computed Tomography (CT) or Magnetic Resonance Imaging (MRI) which produces a picture of the brain that allows identification of such features as volume loss or abnormal structural features such as white matter disease, masses, or vascular abnormalities, which are considered indications of neuronal degeneration, may also be used. Cerebrospinal Fluid (CSF) Tests are also possible, in which the relationship between cerebrospinal fluid amyloid beta (Aβ) and tau, proteins associated with Alzheimer's disease, are quantified. Genetic testing is also available.

“Parkinson's disease”, also referred to as “PD”, is a chronic disease of the central nervous system. Parkinson's disease is characterized by the presence of Lewy bodies and the loss of dopamine-producing neurons in substantia nigra that controls muscle movement. The Lewy body is an abnormal structure found in certain areas of the brain. It contains a protein called α-synuclein, which plays the central role in Parkinson's disease and other diseases involving Lewy bodies, such as dementia with Lewy bodies, multiple system atrophy, and Hallervorden-Spatz disease (The Editors of Encyclopaedia Britannica—Parkinson disease).

Synucleins are small, soluble proteins expressed primarily in neural tissue and in certain tumors (Lavedan C., Genome Res. 8: 871-80, 1998). The family includes three known proteins: alpha-synuclein (α-synuclein), beta-synuclein (β-synuclein), and gamma-synuclein (γ-synuclein). All synucleins have in common a highly conserved alpha-helical lipid-binding motif with similarity to the class-A2 lipid-binding domains of the exchangeable apolipoproteins (Perrin R. J. et al., J. Biol. Chem. 275: 34393-8, 2000).

The alpha- and beta-synuclein proteins are found primarily in brain tissue, where they are seen mainly in presynaptic terminals (Iwai et al., Neuron 14: 467-75, 1995). The gamma-synuclein protein is found primarily in the peripheral nervous system and retina. Mutations in alpha-synuclein are associated with rare familial cases of early-onset Parkinson's disease, and the protein accumulates abnormally in Parkinson's disease, Alzheimer's disease, and several other neurodegenerative illnesses.

“Huntington's Disease”, also referred to as “HD” (HD), also known as Huntington's Chorea, is a progressive disorder of motor, cognitive and psychiatric disturbances. The mean age of onset for this disease is age 35-44 years, although in about 10% of cases, onset occurs prior to age 21, and the average lifespan post-diagnosis of the disease is 15-18 years. Prevalence is about 3 to 7 among 100,000 people of western European descent. HD is an example of a trinucleotide repeat expansion disorder which involves the localized expansion of unstable repeats of sets of three nucleotides and can result in loss of function of the gene in which the expanded repeat resides, a gain of toxic function, or both. Trinucleotide repeats can be located in any part of the gene, including non-coding and coding gene regions. Repeats located within the coding regions typically involve either a repeated glutamine encoding triplet (CAG) or an alanine encoding triplet (CGA). Expanded repeat regions within non-coding sequences can lead to aberrant expression of the gene while expanded repeats within coding regions may cause misfolding and protein aggregation. The exact cause of the pathophysiology associated with the aberrant proteins is often not known. In HD, repeats are inserted within the N terminal coding region of the large cytosolic protein Huntingtin (Htt). Normal Htt alleles contain 15-20 CAG repeats, while alleles containing 35 or more repeats can be considered potentially HD causing alleles and confer risk for developing the disease. In HD, the mutant Htt allele is usually inherited from one parent as a dominant trait.

A genetic test which analyses DNA for the HD mutation by counting the number of CAG repeats in the huntingtin gene are available for diagnosis. Cognitive test and family history may also be considered.

“Amyotrophic lateral sclerosis” (ALS) is a fatal neuromuscular disease which afflicts about 30,000 patients in North America, with 5,000 new cases per year. In ALS, also known as “Lou Gehrig's disease,” muscles of the limbs, speech and swallowing, and respiration weaken and atrophy, due to degeneration of motor nerve cells that supply them from the spinal cord and brain. Half of affected patients are dead within 3 years, with survival over 5 years being less than 20%.

In ALS, the aggregated misfolded proteins are thought to cause progressive killing of brain cells. About 20% of familial (inherited) ALS is associated with mutations in the gene encoding superoxide dismutase 1 (SOD1), an intracellular free radical defence enzyme. Intracellular deposits of aggregated misfolded SOD1 have been observed in familial ALS, and also in the more common non-familial (sporadic) ALS, suggesting that SOD1 aggregation may underlie all ALS.

It will be appreciated by those skilled in the art that the disease can be diagnosed using a genetic test which screens for known mutations. Cognitive test and family history may also be considered.

“Lewy-body dementia” also known as dementia with Lewy bodies, is a type of progressive dementia estimated to affect more than 100,000 people in the UK. Protein deposits, called Lewy bodies, develop in nerve cells in the brain regions involved in thinking, memory and movement (motor control). Symptoms can include cognitive impairment, neurological signs, sleep disorder, and autonomic failure. Lewy bodies are formed from phosphorylated and non-phosphorylated neurofilament proteins; they contain the synaptic protein alpha-synuclein as well as ubiquitin, which is involved in the elimination of damaged or abnormal proteins. In addition to Lewy Bodies, Lewy neurites, which are inclusion bodies in the cell processes of the nerve cells, may also be present.

It will be appreciated by those skilled in the art that the disease can be diagnosed using a genetic test which screens for alpha synuclein mutations. Cognitive/memory tests may also be used.

“Frontotemporal Dementia” (FTD) is a clinical syndrome caused by degeneration of the frontal lobe of the brain and may extend back to the temporal lobe. It is one of three syndromes caused by frontotemporal lobar degeneration, and the second most common early-onset dementia after Alzheimer's disease (BMJ 2013; 347:f4827). Symptoms can be classified (roughly) into two groups which underlie the functions of the frontal lobe: behavioural symptoms (and/or personality change) and symptoms related to problems with executive function. Behavioural symptoms include lethargy and aspontaneity or oppositely disinhibition. Executive function is the cognitive skill of planning and organizing and as such, patients become unable to perform skills that require complex planning or sequencing. In addition, there are specific clinical manifestations of FTD, including: Primary Progressive Aphasia (PPA) and Semantic Dementia (SD). The age of onset is around 40 to 50 years old and the median survival time is seven years. There is currently no treatment for FTD.

By “Transmissible Spongiform Encephalopathies” (TSE's), also termed “prion diseases”, we include a group of progressive, invariably fatal, conditions that affect the brain (“encephalopathies”) and nervous system of many animals, including humans. In humans these diseases include Creutzfeldt-Jakob disease (CJD), Gerstmann-Straussler-Scheinker syndrome (GSS), Fatal Familial Insomnia, and Kuru in humans. In animals the TSE's include sheep scrapie, bovine spongiform encephalopathy (BSE), transmissible mink encephalopathy, and chronic wasting disease of captive mule deer and elk. Prions are the infectious pathogen that causes spongiform encephalopathies. Prions differ significantly from bacteria, viruses and viroids. The dominating hypothesis is that, unlike all other infectious pathogens, infection is caused by an abnormal conformation of the prion protein, which acts as a template and converts normal prion conformations into abnormal conformations. The key characteristic of prion diseases is the formation of an abnormally shaped protein (PrP^{Sc}) from the normal (cellular or non-pathogenic) form of prion protein (PrP^{C}).

It will be appreciated by those skilled in the art that the disease can be diagnosed by a genetic screen and symptomatic diagnosis.

By “Polyglutamine diseases” (polyQ) we include a group of neurodegenerative disorders caused by expanded cytosine-adenine-guanine (CAG) repeats encoding a long polyQ tract in the respective proteins. Proteins containing expanded polyglutamine repeats appear to self-aggregate and, as a result, cause neuronal cell death or degeneration. Non-limiting examples of these diseases are the following: spinobulbar muscular atrophy (SBMA) or Kennedy's disease, caused by expanded polyglutamine repeats in the gene encoding the androgen receptor (AR); Huntington's disease (HD), caused by expanded polyglutamine repeats in the huntingtin gene; spinocerebellar ataxia type 1 (SCA1) caused by increased polyglutamine repeats in the ataxin-1 gene; serpinopathies caused by mutations in serpin genes (serine protease inhibitors); spinocerebellar ataxia type 2 (SCA2) caused by increased polyglutamine repeats in the ataxin-3 gene; Machado-Joseph disease (MJD or SCA3) caused by increased polyglutamine repeats in the ataxin-3 gene; spinocerebellar ataxia type 6 (SCA6) caused by increased polyglutamine repeats in the ataxin-6 gene; spinocerebellar ataxia type 7 (SCA7) caused by increased polyglutamine repeats in the ataxin-7 gene; spinocerebellar ataxia type 17 (SCA17) caused by increased polyglutamine repeats in the ataxin-17 gene; dentatorubral-pallidolusian atrophy (DRPLA); and serpinopathies caused by mutations in the serpin genes. While each of these diseases can be caused by mutations in a distinct protein, they all share a common characteristic, namely aggregation through self-association. The translated polyQ is aggregated in the degenerated neurons leading to the dysfunction and degeneration of specific neuronal subpopulations.

It will be appreciated by those skilled in the art that the disease can be diagnosed by a genetic screen.

Preferably, the calcium channel inhibitor decreases cellular calcium in one or more cell.

By “cellular calcium” we include the concentration of calcium ions inside a given cell. In a preferred embodiment, the cellular calcium is intracellular calcium. Intracellular calcium is generally stored in the mitochondria and the endoplasmic reticulum, and intracellular calcium levels may be relatively low with respect to the extracellular fluid. At rest, most healthy neurons have an intracellular calcium concentration of about 50-100 nM, although this concentration may be higher in pathological situations.

By “decreases cellular calcium” we include that the calcium channel inhibitor may decrease the level of intracellular calcium by a measurable level, for example, by at least 10%, or 20%, or 30%, or 40%, or 50%, or 60%, or 70%, or 80%, or 90%, or 95%, or 99%, or more (i.e. 100%).

In one embodiment, the cell is a cell of the nervous system. In an embodiment, the one or more cell is a cell of the nervous system of an individual—for example, a cell of the central or peripheral nervous system such as a cell of the brain, spinal cord, nerves, sensory neurons and/or ganglia.

Without wishing to be bound by theory, the present inventors hypothesise that the pathology of an amyloid disease of the nervous system and/or neurodegenerative disease or condition, increases intracellular calcium. Thus, in one embodiment, the calcium channel inhibitor according to the invention, leads to a normalization of this increased intracellular calcium to physiological levels, i.e. a decrease from the excess calcium influx existing in the pathological situation.

Preferably, the calcium channel inhibitor is capable of increasing proteolysis. Without wishing to be bound by theory, the inventors hypothesise that proteolysis is increased through the basal mechanism of modulating intracellular calcium concentration.

By “proteolysis” we include the enzymatic process by which proteins are degraded into their component polypeptide or amino acid parts. This generally occurs through protease-mediated hydrolysis of peptide bonds, but can also occur through non-enzymatic methods such as by action of pH, mineral acids and heat. It will be appreciated that proteolysis can be carried out by the proteasome. The proteasome is a sophisticated protease complex designed to carry out selective, efficient and processive hydrolysis of target proteins; it is known to cooperate with ubiquitin, which polymerizes to form a marker for regulated proteolysis in eukaryotic cells. By “proteolysis” we also include autophagy, a process involved in the proteolytic degradation of cellular macromolecules in lysosomes.

By “increasing proteolysis” we include an enhanced rate of proteolysis of damaged/aggregated/misfolded proteins in a cell, group of cells, tissue, animal model or in an individual. For example, proteolysis may have increased following the administration of a calcium channel inhibitor, relative to the level of proteolysis in a non-treated cell, group of cells, tissue, animal model or individual, or in a cell, group of cells, tissue, animal model or individual administered a compound which does not have an effect on calcium channels, such as a control compound, for example Dimethyl sulfoxide (DMSO). We include that proteolysis has been increased by a measurable level, for example, by at least 10%, or 20%, or 30%, or 40%, or 50%, or 60%, or 70%, or 80%, or 90%, or 95%, or 99% or more (i.e. 100%). Proteolysis may be increased through activation of the proteasome.

In a preferred embodiment, the calcium channel inhibitor is capable of increasing proteolysis of proteins involved in the formation of amyloid deposits. For example, following administration with the calcium channel inhibitor of the invention, proteolysis of an amyloidogenic polypeptide may be increased relative to the proteolysis of a non-amyloidogenic polypeptide. Examples of amyloidogenic peptides include, but are not limited to, Aβ peptide, Tau, α-synuclein, Huntingtin, SOD, FUS, TDP-43, and Prion Protein.

Levels of proteolysis can be assessed using methods known in the art, such as by measuring the turnover of a specific substrate and/or activity-based probe incorporation.

Preferably, the calcium channel inhibitor is capable of activating the proteasome. By activating the proteasome, the calcium channel inhibitor may increase the rate at which the proteasome (such as the multi-subunit 26S proteasome) recognizes, unfolds, and/or degrades ubiquitinated substrates into small peptides.

Methods for measuring the activity of the proteasome are known in the art, for example, a common assessment of proteasome activity is done by measuring the hydrolysis of a fluorogenic peptidyl substrate.

Preferably, the calcium channel inhibitor is capable of reducing protein aggregation and/or protein misfolding. For example, by increasing proteolysis, the calcium channel inhibitor may be capable of decreasing protein aggregation and/or misfolding of the aggregation-prone proteins selected from the group comprising: Aβ peptide, Tau, α-synuclein, Huntingtin, SOD1, FUS, TDP-43, and Prion Protein.

In an embodiment, the protein aggregation and/or misfolding is present in a cell of the nervous system. For example, a cell of the central or peripheral nervous system, such as a cell of the brain, spinal cord, nerves, sensory neurons and/or ganglia. In a further embodiment, the protein aggregation and/or misfolding is present in the brain, or in a brain cell.

The calcium channel inhibitor may act directly on the calcium channel in order to cause a reduction in protein aggregation and/or protein misfolding. Alternatively, the calcium channel inhibitor may have an indirect effect of the calcium channel and therefore on protein aggregation and/or misfolding.

Preferably the calcium channel inhibitor is capable of reducing the number and/or size of amyloid deposits in the nervous system of the individual. In one embodiment, the amyloid deposit are amyloid plaques, such as Aβ plaques.

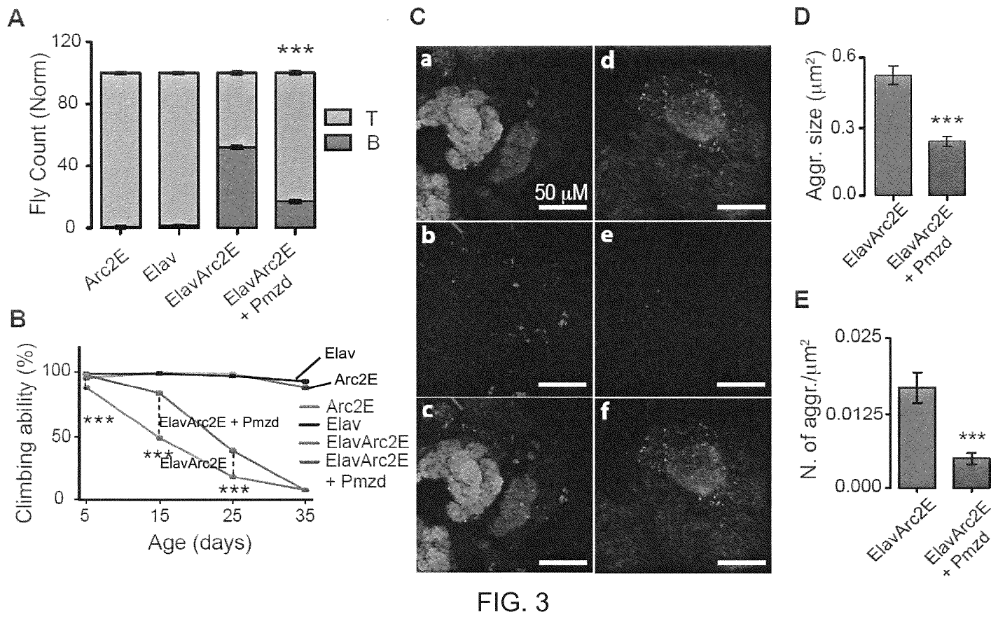

As can be seen in the accompanying Examples, the inventors demonstrated that a calcium channel inhibitor was capable of causing a reduction of Aβ plaque burden in an animal model of Alzheimer's Disease. Both a Drosophila model overexpressing Aβ, and a control strain was treated with a calcium channel inhibitor (pimozide) and Aβ histology (plaque burden) was evaluated. After dissecting out the brains and staining for Aβ, a clear reduction was seen in the frequency, and size of Aβ aggregates in the treated group of flies (as shown, for example, in FIG. 3).

In an alternative embodiment, the amyloid deposits are neurofibrillary tangles comprising the protein Tau.

In an alternative embodiment, the amyloid deposits are intracellular inclusions comprising the protein Huntingtin.

In an alternative embodiment, the amyloid deposits comprise the prion protein.

In an alternative embodiment, the amyloid deposits comprise intracellular deposits of aggregated misfolded SOD1 protein.

In an alternative embodiment, the amyloid deposits are Lewy bodies or protein aggregates comprising the protein alpha-synuclein.

Preferably, the calcium channel inhibitor is capable of preventing the loss of and/or restoring cognitive function.

As shown in the Examples, the inventors have observed that the restoration in cognition, observed through measurement of gamma oscillations, in mouse in vitro occurs within minutes of administration of the calcium channel inhibitor. Without being bound by theory, the inventors hypothesise that the gain-of-function of cognition-relevant network activity (gamma oscillations), the rescue of the underlying cellular synchronization, and the re-establishment of correct excitatory/inhibitory balance in the network observed are therefore independent of proteasome activation.

By “cognitive function” we include the ability to perform mental tasks, such as thinking, learning, judging, remembering, computing, controlling motor functions, and the like. Cognitive function may be assessed using neuropsychological tests of global cognitive function, such as memory, language, visuospatial ability, processing speed and attention/working memory/executive function.

Preferably, cognitive function is assessed by determining neuronal oscillations in the brain of the individual. It will be appreciated that these are cognition-relevant neuronal oscillations.

By “neuronal oscillations” we include the rhythmic electrical activity in neuronal networks. We include the rhythmic and/or repetitive electrical activity generated spontaneously and in response to stimuli by neural tissue in the nervous system. Also known as “neural oscillations”, this electrical activity can be characterized by frequency, amplitude and phase. The first-discovered and best-known frequency band is alpha activity that can be detected from the occipital lobe during relaxed wakefulness and which increases when the eyes are closed. Other frequency bands are: delta, theta, beta and gamma. In the healthy brain, electrical oscillations in the gamma-frequency band (20-80 Hz) in hippocampal and neocortical networks, play an important role in learning, memory and cognition.

Neuronal oscillations can be measured by electrophysiological recording techniques such as electroencephalography (EEG). EEG is a non-invasive electrophysiological monitoring method which can record electrical activity of the brain. Typically, electrodes are placed along the scalp, although invasive electrodes are sometimes used such as in electrocorticography. EEG measures voltage fluctuations resulting from ionic current within the neurons of the brain. In clinical contexts, EEG refers to the recording of the brain's spontaneous electrical activity over a period of time, as recorded from multiple electrodes placed on the scalp. Magnetoencephalography (MEG) is a further technique for mapping brain activity, which works by recording magnetic fields produced by electrical currents occurring naturally in the brain, using very sensitive magnetometers.

Oscillatory activity is observed throughout the central nervous system at all levels of organization. Three different levels have been widely recognized: the micro-scale (activity of a single neuron), the meso-scale (activity of a local group of neurons) and the macro-scale (activity of different brain regions). Neurons generate action potentials resulting from changes in the electric membrane potential. Oscillatory activity in single neurons can also be observed in sub-threshold fluctuations in membrane potential. These rhythmic changes in membrane potential do not reach the critical threshold and therefore do not result in an action potential.

Preferably, the neuronal oscillations are in the gamma-frequency and/or theta-frequency range. As a rhythmic electrical brain activity, the generation and maintenance of gamma oscillations is dependent on the synchronization of action potential firing of different cell classes and the tightly regulated balance of excitation and inhibition in the neuronal circuitry. Gamma oscillations have been suggested to underlie higher cognitive functions, such as sensory perception, attention, and memory, and are known to be significantly degraded in Alzheimer Disease (AD) patients, who suffer from deficiencies in their cognitive faculties. It has been shown that increased Aβ levels in AD mouse models result in disrupted timing of evoked action potentials (Kurudenkandy et al., Journal of Neuroscience 20 Aug. 2014, 34 (34) 11416-11425). Indeed, clinical data shows that there is a strong correlation between gamma oscillations measured in patient EEG or MEG, and the progression of AD and impairment of cognition. Therefore, gamma oscillations can serve as a functional biomarker for diagnosis in the clinic.

It will be appreciated by those skilled in the art that gamma oscillations in acute brain slice preparations can be induced pharmacologically and serve as a model system to study the underlying circuitry and pathological mechanisms.

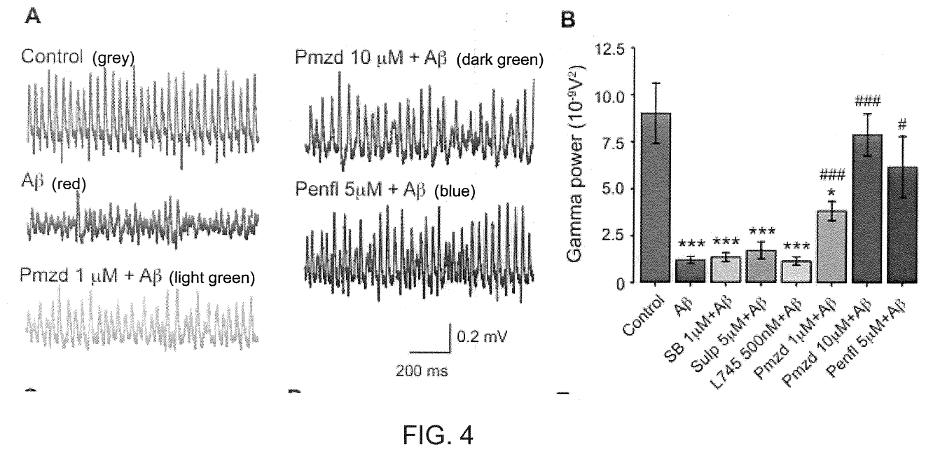

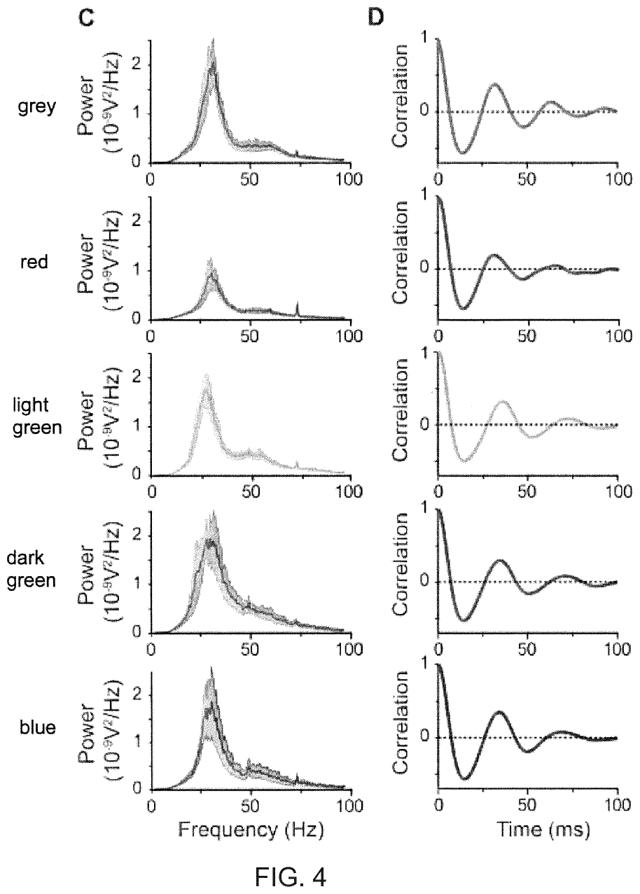

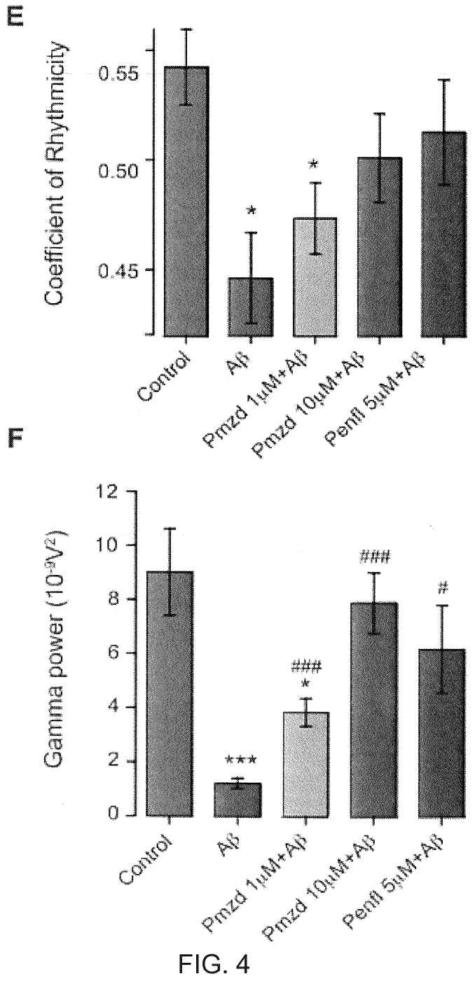

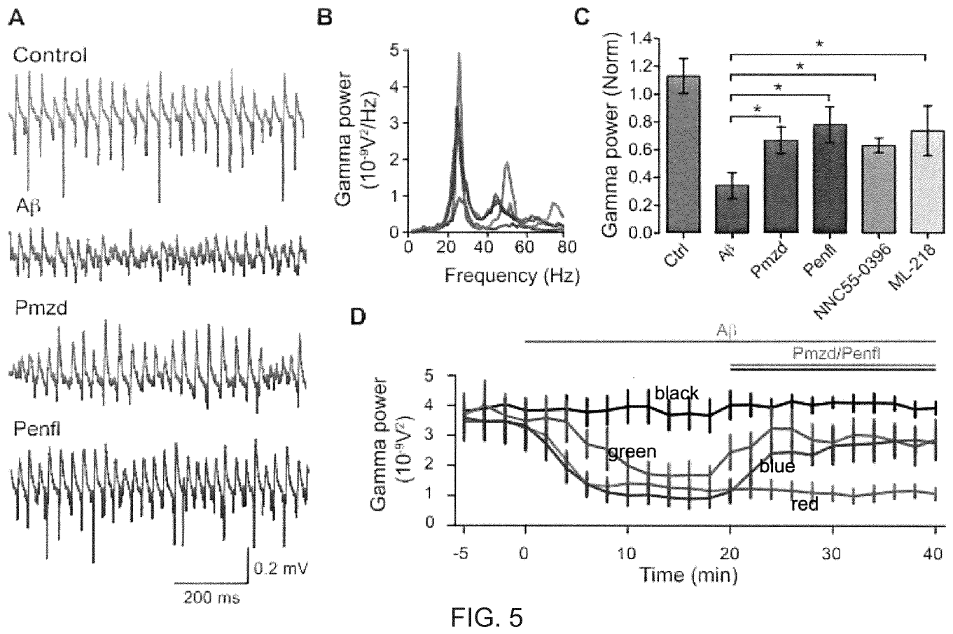

As demonstrated in the accompanying Examples, calcium channel inhibitors (pimozide and penfluridol) are able to prevent the Aβ-induced degradation of gamma oscillations observed in local field recordings in the mouse hippocampal network (see, for example, FIG. 4). Further, the results show that calcium channel inhibitors (pimozide and penfluridol) are able to rescue gamma oscillations previously degraded by Aβ (see, for example, FIG. 5).

Accordingly, in an embodiment, the calcium channel inhibitor is capable of reducing and/or rescuing gamma oscillations previously degraded by protein aggregates of misfolded proteins.

Preferably, the calcium channel inhibitor is capable of fully or partially restoring action potential synchronization in the nervous system of the individual.

By “action potential synchronization” we include a change in a neuron's membrane potential caused by ions flowing in and out of the neuron which occurs at substantially the same time as it occurs in surrounding neurons.

By “restoring action potential synchronization” we include fully or partially restoring action potential synchronization to physiological levels or normal levels. It will be appreciated that action potential desynchronization is a consequence of the pathology of an amyloid disease of the nervous system, or a neurodegenerative disease and/or condition.

Preferably, action potential desynchronization is caused by protein aggregation and/or protein misfolding. As discussed above, it has been shown previously that accumulation protein aggregates or misfolded proteins, such as Aβ peptides, can induce the degradation of gamma oscillations.

Preferably, the calcium channel inhibitor comprises a voltage gated calcium channel (VGCC) inhibitor.

By “voltage gated calcium channel (VGCCs)” we include a family of molecules that allow cells to couple electrical activity to intracellular calcium signalling. The opening and closing of these channels by depolarizing stimuli, such as action potentials, allows calcium ions to enter neurons down a steep electrochemical gradient, producing transient intracellular calcium signals. Calcium channels can be divided into ligand bound or voltage gated calcium channels (VGCC). VGCCs can be further divided into two groups of channels: high-voltage activated calcium channels (termed L, N, P/Q, and R-types) and low-voltage activated calcium channels (termed T-types). Voltage-gated calcium channels are made up of several subunits. The alpha (α)1 subunit forms the pore for the import of extracellular calcium ions and, though regulated by the other subunits, is primarily responsible for the pharmacological properties of the channel. The Alpha-1 subunit also determines the type of calcium channel. The Beta-, Alpha-2-Delta, and Gamma subunits, present in only some types of calcium channels, are auxiliary subunits that play secondary roles in the channel (Simms B A, Zamponi G W. Neuron. 2014 Apr. 2; 82(1):24-45).

VGCCs are defined by their α subunits sub-categorized as the L-types CaV1.1 (α1S), 1.2 (α1C), 1.3 (α1D), 1.4 (α1F), the P/Q-type CaV2.1 (α1A), the N-type CaV2.2 (α1B), the R-type (α1E) and the T-types as CaV3.1 (α1G), CaV3.2 (α1H) and CaV3.3 (α1I). L-type calcium channels possess at least two additional subunits that may help differentiate them from the T-type calcium channel (Simms B A, Zamponi G W. Neuron. 2014 Apr. 2; 82(1):24-45).

By “voltage gated calcium channel (VGCC) inhibitor” we include the meaning of any agent which reduces and/or prevents the activity of a VGCC. In an embodiment, the calcium channel inhibitor of the invention is selective for VGCCs, meaning that the calcium channel inhibitor substantially reduces and/or prevents the activity of VGCCs to a greater extent than it inhibits the activity of other types of VGCCs, such as ligand bound calcium channels. In the context of the present invention a VGCC inhibitor can completely block, partially block or decrease the flux of calcium ions through the VGCC, for example by at least 10%, or 20%, or 30%, or 40%, or 50%, or 60%, or 70%, or 80%, or 90%, or 95%, or 99%, or 100%.

Preferably, the VGCC inhibitor comprises a T-type VGCC inhibitor.

By “T-type VGCC” we include low-voltage activated calcium channels that open during membrane depolarization. The primary function of the T-type voltage gated calcium channel is to allow passage of ions, in this case calcium, through the membrane when the channel is activated. T-type calcium channels have been identified in various mammals including humans. The T-type class is characterized by fast inactivation (transient) and small conductance (tiny) and is composed of three members due to the different main pore-forming al subunits: Cav3.1 (a1 G), Cav3.2 (a1H) and Cav3.3 (a1I). Cav3.1 and Cav3.3 are mainly expressed in the brain, while Cav3.2 is found in brain and peripheral tissues (e.g., heart, kidney, liver). As a member of the Cav3 subfamily of VGCCs, the function of the T-type channel is important for the repetitive firing of action potentials in cells with rhythmic firing patterns such as cardiac muscle cells and neurons in the thalamus of the brain. T-type calcium channels are predominantly found in neurons but have been found in other cells including cardiac myocytes, pacemaker cells, glial cells, fibroblasts, osteoblasts, retinal cells, and adrenocortical cells (Simms B A, Zamponi G W. Neuron. 2014 Apr. 2; 82(1):24-45).

By “T-type VGCC inhibitor” we include the meaning of any natural or synthetic agent which substantially inhibits the activity of a T-type VGCC. In an embodiment, the calcium channel inhibitor of the invention is selective for T-type VGCCs, meaning that the calcium channel inhibitor of the invention inhibits the activity of T-type VGCCs to a greater extent than it inhibits the activity of other types of VGCCs, such as L, N, P/Q, and R-type VGCCs. In the context of the present invention a T-type VGCC inhibitor can completely block, partially block or decrease the flux of calcium ions through the T-type VGCC by at least 10%, or 20%, or 30%, or 40%, or 50%, or 60%, or 70%, or 80%, or 90%, or 95%, or 99%, or 100%.

In an embodiment, the T-type VGCC inhibitor inhibits a T-type VGCC subtype selected from the group comprising: CaV3.1 (α1G), CaV3.2 (α1H) and CaV3.3 (α1I). In one embodiment, the T-type VGCC inhibitor inhibits a CaV3.1 (α1G) T-type VGCC. In one embodiment, the T-type VGCC inhibitor inhibits a CaV3.2 (α1H) T-type VGCC. In one embodiment, the T-type VGCC inhibitor inhibits a CaV3.3 (α1I) T-type VGCC.

In an embodiment, the VGCC inhibitor inhibits a T-type VGCC and an L-type VGCC.

By “L-type VGCC” we include the meaning of a long-opening high-voltage-gated calcium channel. L-type channels comprise several subunits including CaV1.1 (α1S), CaV1.2 (α1C), CaV1.3 (α1D), CaV1.4 (α1F).

In an embodiment, the calcium channel inhibitor of the invention is selective for L-type calcium channels, meaning that the calcium channel inhibitor of the invention inhibits the activity of L-type VGCCs to a greater extent than it inhibits the activity of any other type of VGCCs, such as N, P/Q, R and T-type VGCCs. In the context of the present invention, the inhibitor can completely block, partially block or decrease the flux of calcium ions through an L-type VGCC by at least 10%, or 20%, or 30%, or 40%, or 50%, or 60%, or 70%, or 80%, or 90%, or 95%, or 99%, or 100%.

In an alternative embodiment, the calcium channel inhibitor of the invention does not inhibit L-type calcium channels. As shown in the accompanying Examples, using the specific L-type calcium channel inhibitor Verapamil, the inventors have found that no reduction of size and frequency of amyloid-beta peptide plaques was observed in the mushroom body of fly brain, no gain-of-function regarding the ability shown in the climbing test is observed in fly, and no gain-of-function regarding a rescue of the amyloid-beta peptide-reduced cognition-relevant gamma oscillations are observed in mouse hippocampus.

In an embodiment, the calcium channel inhibitor of the invention is any one of a small molecule, a polypeptide, a peptide, an antibody, a polynucleotide, a peptidomimetic, a natural product, a carbohydrate or an aptamer.

In one embodiment, the calcium channel inhibitor is a small molecule.

The term “small molecule” includes small organic molecules. Suitable small molecules may be identified by methods such as screening large libraries of compounds (Beck-Sickinger & Weber (2001) Combinational Strategies in Biology and Chemistry (John Wiley & Sons, Chichester, Sussex); by structure-activity relationship by nuclear magnetic resonance (Shuker et al (1996) “Discovering high-affinity ligands for proteins: SAR by NMR. Science 274: 1531-1534); encoded self-assembling chemical libraries Melkko et al (2004) “Encoded self-assembling chemical libraries.” Nature Biotechnol. 22: 568-574); DNA-templated chemistry (Gartner et al (2004) “DNA-templated organic synthesis and selection of a library of macrocycles. Science 305: 1601-1605); dynamic combinatorial chemistry (Ramstrom & Lehn (2002) “Drug discovery by dynamic combinatorial libraries.” Nature Rev. Drug Discov, 1: 26-36); tethering (Arkin & Wells (2004) “Small-molecule inhibitors of protein-protein interactions: progressing towards the dream. Nature Rev. Drug Discov. 3: 301-317); and speed screen (Muckenschnabel et al (2004) “SpeedScreen: label-free liquid chromatography-mass spectrometry-based high-throughput screening for the discovery of orphan protein ligands.” Anal. Biochem. 324: 241-249). Typically, small organic molecules will have a dissociation constant for the polypeptide in the nanomolar range, particularly for antigens with cavities. The benefits of most small organic molecule binders include their ease of manufacture, lack of immunogenicity, tissue distribution properties, chemical modification strategies and oral bioavailability. Small molecules with molecular weights of less than 5000 daltons are preferred, for example less than 400, 3000,2000, or 1000 daltons, or less than 500 daltons.

In an embodiment, the calcium channel inhibitor is capable of crossing the blood-brain barrier (BBB). By “crossing the blood-brain barrier” we include the ability of a compound to transit to the brain in detectable amounts following systemic administration. The ability of a compound to cross the BBB can be assessed using methods known in the art. In general, compounds that cross the BBB will have molecular weights of less than 400 daltons and a degree of lipid solubility. Alternatively, the compound can be modified to improve its ability to cross the blood-brain barrier, and in an alternative embodiment, the compound can be co-administered with an additional agent that improves the ability of the compound to cross the BBB. Alternatively, precise delivery of the pharmacological agent into specific sites of the brain, can be conducted using stereotactic microinjection techniques.

It will be appreciated that a calcium channel inhibitor that is capable of crossing the BBB will do so to achieve a minimum effective concentration.

Preferably, the calcium channel inhibitor is selected from the group comprising: a diphenylbutylpiperidine, a benzimidazole, 3-azabicyclo hexane, a quinazolin-2-one, a piperidine, a pyridine, a pyrazine, a piperazine (for example, a di-tert-butylphenyl piperazine or a piperazinylalkylpyrazole), an amino acid (for example, a (1-H-indol-3y1) ethylamine amino acid, a 3-(phenyl)acrylate ethylamine amino acid or an α,α spirocyclic amino acid), an N-piperidinyl acetamide, a 4-aminomethyl-piperidine, a bicyclic pyrimidine (for example, a 1,4-bisaminomethyl-cyclohexyl or a 4-(aminomethyl)-cyclohexylamine), a dihydropyrimidine, a dihydropyrimidone, a sulfonamide derivative, a substituted thiazole, a spiroazetidine, a spiroazetidinone, an oxadiazole (for example, a 5-methyl-oxadiazole), a benzhydryl, a benzenesulfonamide, a 3,4-dihydroquinazoline, a 2,4-dioxo-tetrahydroquinazoline, a 4-oxo-2-thioxo-tetrahydroquinazoline, a 1,3-dioxoisoindole, a 3-oxo-isoindoline, a morpholin-2-one, a 2-guanidino-thiazole, a 2-imino-1,3-thiazoline; a pyrrolidine (and open chain analogues thereof), Ethosuximide, Trimethadione, Zonisamide, Amlodipine, Aranidipine, Azelnidipine, Barnidipine, Benidipine, Efonidipine, Mibefradil, Nicardipine, Nimodipine, Lomerizine, A1048400, KYS05044, ML218, NNC 55-0396, RQ-00311610, TTA-A2, TTA-P2, VH04 and Z941/944.

In particular embodiments, the calcium channel inhibitor is selected from those shown in the following table:

| | |
|---|---|
| 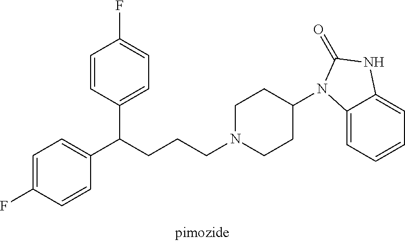 [SMILES: O=c1[nH]c2ccccc2n1C1CCN(CCCC(c2ccc(F)cc2)c2ccc(F)cc2)CC1] | |
| 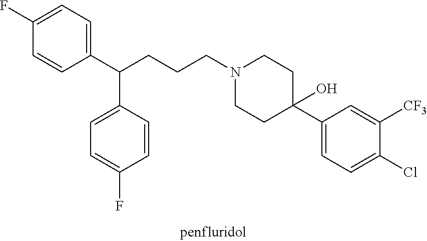 [SMILES: OC1(c2ccc(Cl)c(C(F)(F)F)c2)CCN(CCCC(c2ccc(F)cc2)c2ccc(F)cc2)CC1] | |
| 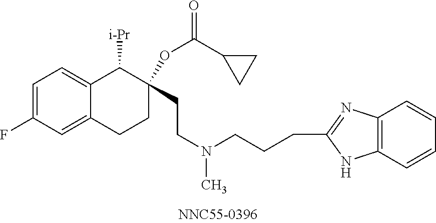 [SMILES: CC(C)[C@H]1c2ccc(F)cc2CC[C@@]1(CCN(C)CCCc1nc2ccccc2[nH]1)OC(=O)C1CC1] | |
| 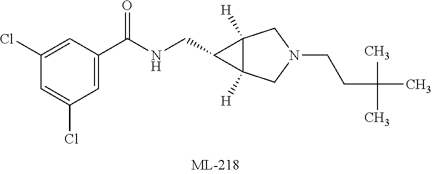 [SMILES: CC(C)(C)CCN1C[C@@H]2[C@@H](CNC(=O)c3cc(Cl)cc(Cl)c3)[C@@H]2C1] | |
| 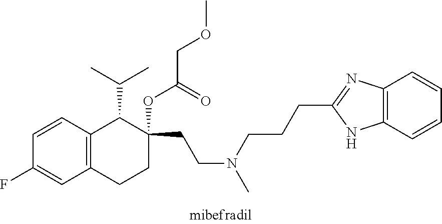 [SMILES: COCC(=O)O[C@]1(CCN(C)CCCc2nc3ccccc3[nH]2)CCc2cc(F)ccc2[C@@H]1C(C)C] | |
| 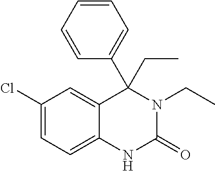 [SMILES: CCN1C(=O)Nc2ccc(Cl)cc2C1(CC)c1ccccc1] | |
| 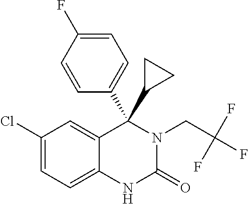 [SMILES: O=C1Nc2ccc(Cl)cc2[C@@](c2ccc(F)cc2)(C2CC2)N1CC(F)(F)F] | TTA-Q3 |
| 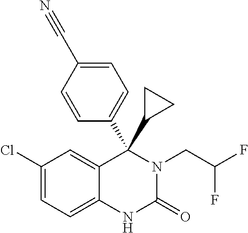 [SMILES: N#Cc1ccc([C@@]2(C3CC3)c3cc(Cl)ccc3NC(=O)N2CC(F)F)cc1] | TTA-Q4 |
| 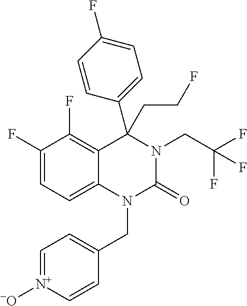 | |
| 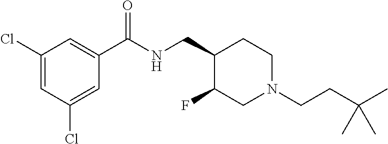 [SMILES: CC(C)(C)CCN1CC[C@H](CNC(=O)c2cc(Cl)cc(Cl)c2)[C@H](F)C1] | TTA-P1 |
| 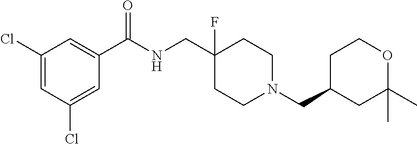 [SMILES: CC1(C)C[C@@H](CN2CCC(F)(CNC(=O)c3cc(Cl)cc(Cl)c3)CC2)CCO1] | TTA-P2 |
| 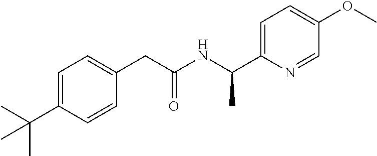 [SMILES: COc1ccc([C@@H](C)NC(=O)Cc2ccc(C(C)(C)C)cc2)nc1] | TTA-A1 |
| 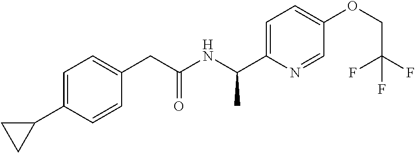 [SMILES: C[C@@H](NC(=O)Cc1ccc(C2CC2)cc1)c1ccc(OCC(F)(F)F)cn1] | TTA-A2 |
| 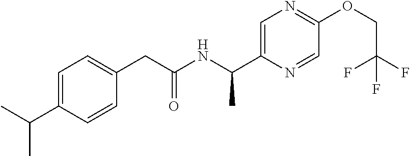 [SMILES: CC(C)c1ccc(CC(=O)N[C@H](C)c2cnc(OCC(F)(F)F)cn2)cc1] | |
| 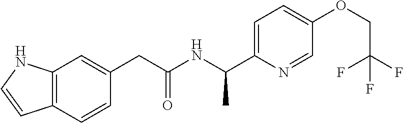 [SMILES: C[C@@H](NC(=O)Cc1ccc2cc[nH]c2c1)c1ccc(OCC(F)(F)F)cn1] | |
| 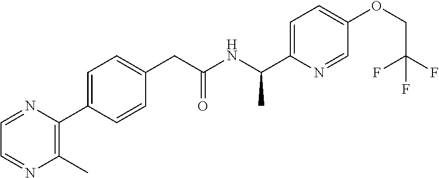 [SMILES: Cc1nccnc1-c1ccc(CC(=O)N[C@H](C)c2ccc(OCC(F)(F)F)cn2)cc1] | TTA-A8 |
| 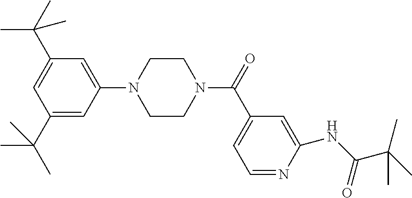 [SMILES: CC(C)(C)C(=O)Nc1cc(C(=O)N2CCN(c3cc(C(C)(C)C)cc(C(C)(C)C)c3)CC2)ccn1] | |
| 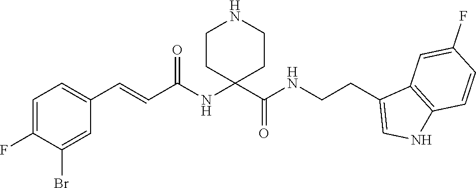 [SMILES: O=C(/C=C/c1ccc(F)c(Br)c1)NC1(C(=O)NCCc2c[nH]c3ccc(F)cc23)CCNCC1] | |
| 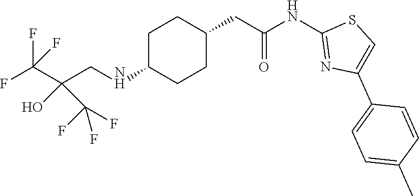 [SMILES: Cc1ccc(-c2csc(NC(=O)C[C@H]3CC[C@@H](NCC(O)(C(F)(F)F)C(F)(F)F)CC3)n2)cc1] | |
| 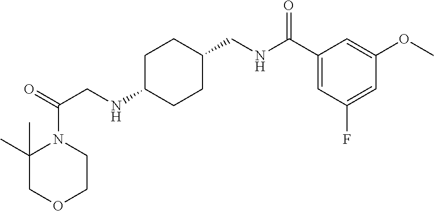 | |
| 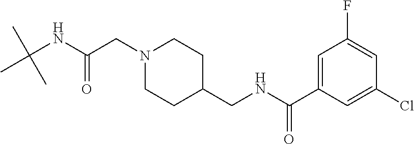 [SMILES: CC(C)(C)NC(=O)CN1CCC(CNC(=O)c2cc(F)cc(Cl)c2)CC1] | |
| 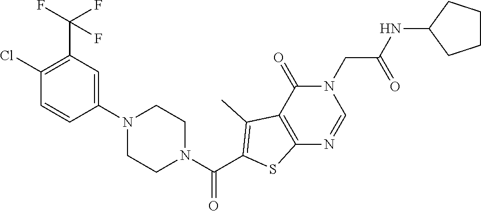 [SMILES: Cc1c(C(=O)N2CCN(c3ccc(Cl)c(C(F)(F)F)c3)CC2)sc2ncn(CC(=O)NC3CCCC3)c(=O)c12] | |
| 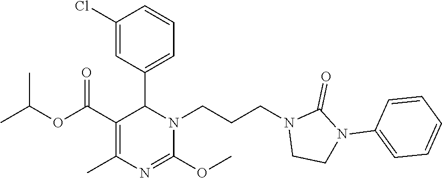 [SMILES: COC1=NC(C)=C(C(=O)OC(C)C)C(c2cccc(Cl)c2)N1CCCN1CCN(c2ccccc2)C1=O] | |
| 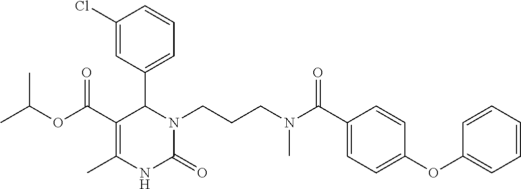 [SMILES: CC1=C(C(=O)OC(C)C)C(c2cccc(Cl)c2)N(CCCN(C)C(=O)c2ccc(Oc3ccccc3)cc2)C(=O)N1] | |
| 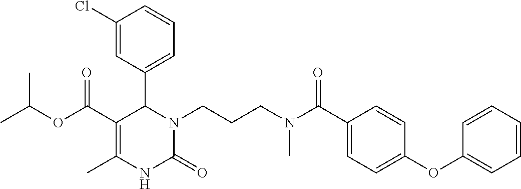 [SMILES: CC1=C(C(=O)OC(C)C)C(c2cccc(Cl)c2)N(CCCN(C)C(=O)c2ccc(Oc3ccccc3)cc2)C(=O)N1] | |
| 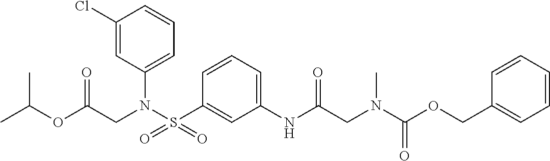 [SMILES: CC(C)OC(=O)CN(c1cccc(Cl)c1)S(=O)(=O)c1cccc(NC(=O)CN(C)C(=O)OCc2ccccc2)c1] | |
| 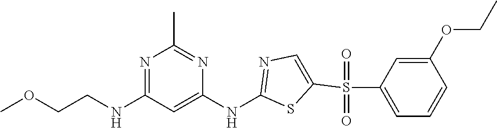 [SMILES: CCOc1cccc(S(=O)(=O)c2cnc(Nc3cc(NCCOC)nc(C)n3)s2)c1] | |
| 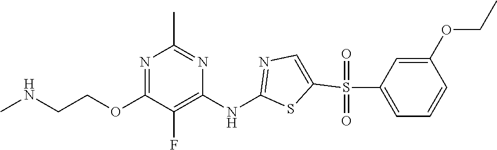 [SMILES: CCOc1cccc(S(=O)(=O)c2cnc(Nc3nc(C)nc(OCCNC)c3F)s2)c1] | |
| 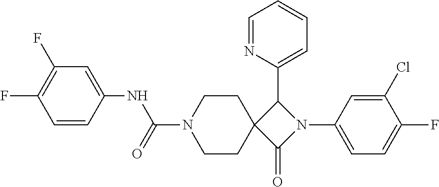 [SMILES: O=C(Nc1ccc(F)c(F)c1)N1CCC2(CC1)C(=O)N(c1ccc(F)c(Cl)c1)C2c1ccccn1] | |
| 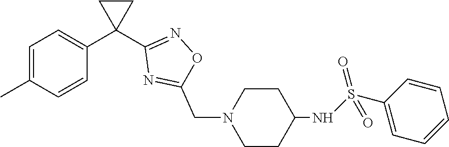 [SMILES: Cc1ccc(C2(c3noc(CN4CCC(NS(=O)(=O)c5ccccc5)CC4)n3)CC2)cc1] | |
| 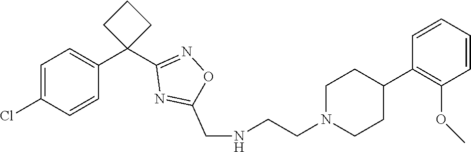 [SMILES: COc1ccccc1C1CCN(CCNCc2nc(C3(c4ccc(Cl)cc4)CCC3)no2)CC1] | |
| 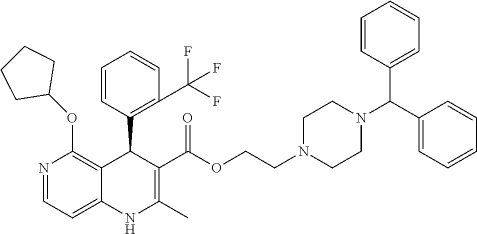 [SMILES: CC1=C(C(=O)OCCN2CCN(C(c3ccccc3)c3ccccc3)CC2)[C@H](c2ccccc2C(F)(F)F)c2c(ccnc2OC2CCCC2)N1] | |
| 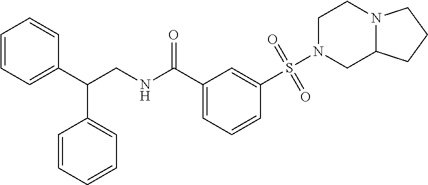 [SMILES: O=C(NCC(c1ccccc1)c1ccccc1)c1cccc(S(=O)(=O)N2CCN3CCCC3C2)c1] | |
| 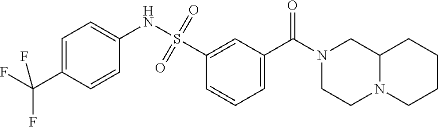 [SMILES: O=C(c1cccc(S(=O)(=O)Nc2ccc(C(F)(F)F)cc2)c1)N1CCN2CCCCC2C1] | |
| 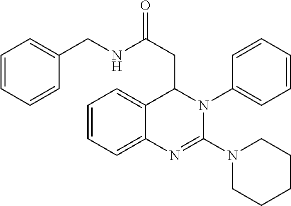 [SMILES: O=C(CC1c2ccccc2N=C(N2CCCCC2)N1c1ccccc1)NCc1ccccc1] | KYSO5001 |
| 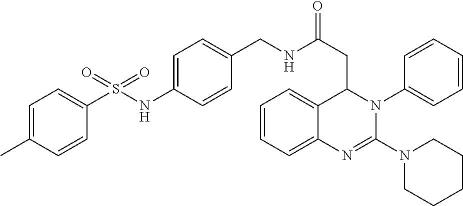 [SMILES: Cc1ccc(S(=O)(=O)Nc2ccc(CNC(=O)CC3c4ccccc4N=C(N4CCCCC4)N3c3ccccc3)cc2)cc1] | KYSO5041 |
| 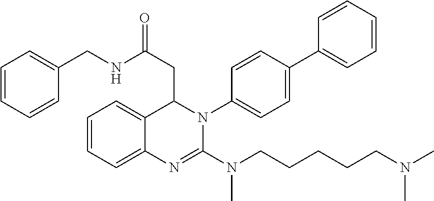 [SMILES: CN(C)CCCCCN(C)C1=Nc2ccccc2C(CC(=O)NCc2ccccc2)N1c1ccc(-c2ccccc2)cc1] | KYSO5090 |
| 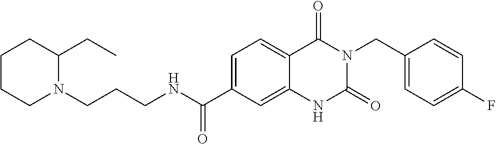 [SMILES: CCC1CCCCN1CCCNC(=O)c1ccc2c(=O)n(Cc3ccc(F)cc3)c(=O)[nH]c2c1] | |
| 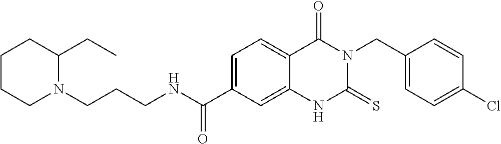 [SMILES: CCC1CCCCN1CCCNC(=O)c1ccc2c(=O)n(Cc3ccc(Cl)cc3)c(=S)[nH]c2c1] | |
| 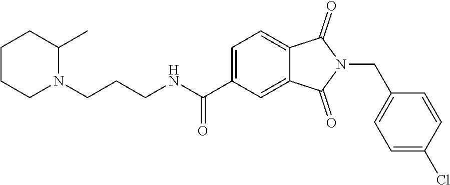 [SMILES: CC1CCCCN1CCCNC(=O)c1ccc2c(c1)C(=O)N(Cc1ccc(Cl)cc1)C2=O] | 24 |
| 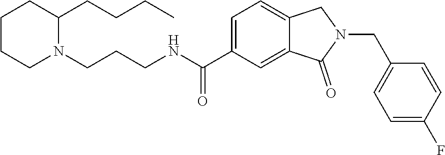 [SMILES: CCCCC1CCCCN1CCCNC(=O)c1ccc2c(c1)C(=O)N(Cc1ccc(F)cc1)C2] | |
| 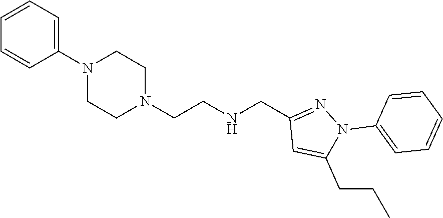 [SMILES: CCCc1cc(CNCCN2CCN(c3ccccc3)CC2)nn1-c1ccccc1] | |
| 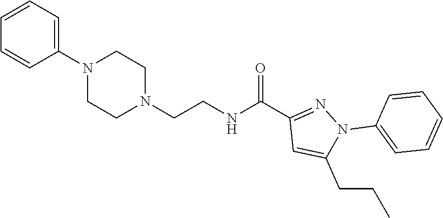 [SMILES: CCCc1cc(C(=O)NCCN2CCN(c3ccccc3)CC2)nn1-c1ccccc1] | |
| 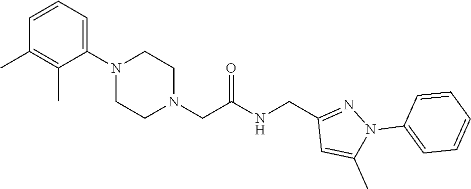 [SMILES: Cc1cccc(N2CCN(CC(=O)NCc3cc(C)n(-c4ccccc4)n3)CC2)c1C] | |
| 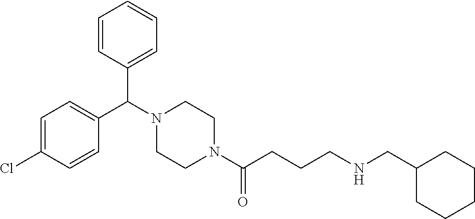 [SMILES: O=C(CCCNCC1CCCCC1)N1CCN(C(c2ccccc2)c2ccc(Cl)cc2)CC1] | |
| 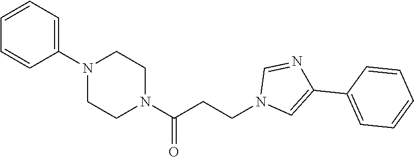 [SMILES: O=C(CCn1cnc(-c2ccccc2)c1)N1CCN(c2ccccc2)CC1] | |
| 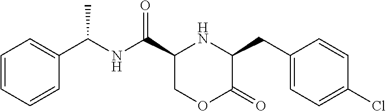 [SMILES: C[C@H](NC(=O)[C@@H]1COC(=O)[C@H](Cc2ccc(Cl)cc2)N1)c1ccccc1] | |
| 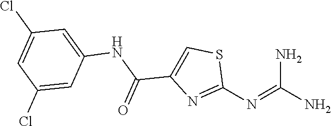 [SMILES: NC(N)=Nc1nc(C(=O)Nc2cc(Cl)cc(Cl)c2)cs1] | |
| 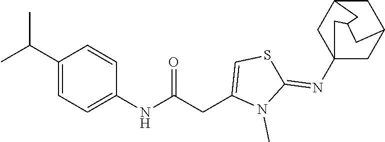 [SMILES: CC(C)c1ccc(NC(=O)Cc2cs/c(=N\C34CC5CC(CC(C5)C3)C4)n2C)cc1] | |
| 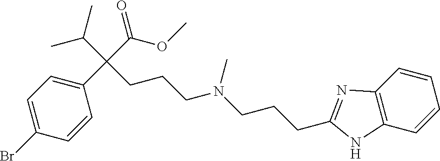 [SMILES: COC(=O)C(CCCN(C)CCCc1nc2ccccc2[nH]1)(c1ccc(Br)cc1)C(C)C] | |
| 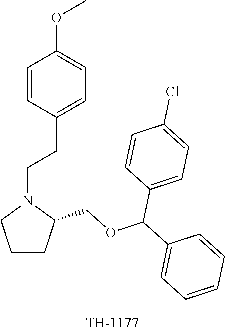 [SMILES: COc1ccc(CCN2CCC[C@H]2COC(c2ccccc2)c2ccc(Cl)cc2)cc1] | |
| 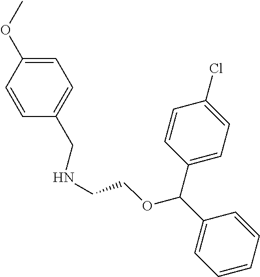 | |
| 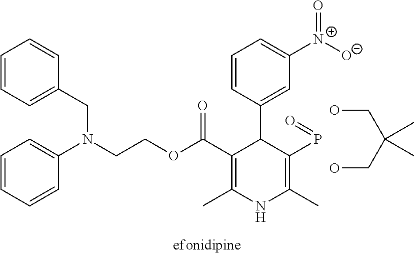 | |

In particular embodiments, the calcium channel inhibitor is selected from the group comprising: pimozide; penfluridol; NNC55-0396; ML-218; mibefradil; efonidipine; TTA-Q3; TTA-Q4; TTA-P1; TTA-P2; TTA-A1; TTA-A2; TTA-A8; KYSO5001; KYSO5041; KYSO5090 and TH-1177.

For the avoidance of doubt, in the case of a discrepancy between the name of the compound and the structure drawn in this specification, the structure should prevail.

Preferably, the calcium channel inhibitor is one or more selected from the group comprising: pimozide, penfluridol, NNC55-0396 and ML-218. In an alternative embodiment, the calcium channel inhibitor is not pimozide. In an alternative embodiment, the calcium channel inhibitor is not niguldipine. In an alternative embodiment, the calcium channel inhibitor is not nicardipine. In an alternative embodiment, the calcium channel inhibitor is not amiodarone. In an alternative embodiment, the calcium channel inhibitor is not loperamide.

As can be seen from the accompanying Examples, the inventors tested additional T-type calcium channel inhibitors, NNC55-0396 and ML-218, each with its own distinct chemical structure, unrelated to pimozide or penfluridol. Both additional T-type calcium channel inhibitors were able to rescue gamma oscillation power after degradation by Aβ (see, for example, FIG. 5).

Further examples of T-type calcium channel inhibitors of the invention include Ethosuximide, Trimethadione, Zonisamide, Amlodipine, Aranidipine, Azelnidipine, Barnidipine, Benidipine, Efonidipine, Mibefradil, Nicardipine, Nimodipine, Lomerizine, A1048400, KYS05044, ML218, NNC 55-0396, RQ-00311610, TTA-A2, TTA-P2, VH04 and Z9411944 (Kopecky et al., Pflugers Arch—Eur J Physiol (2014) 466:757-765, herein incorporated by reference).

The calcium channel inhibitors of the invention may be administered in a number of ways. Methods of administered include, but are not limited to, intradermal, intramuscular, intraperitoneal, intravenous, subcutaneous, intranasal, epidural, and oral routes. The inhibitor may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

For instance, systemic administration may be required in which case the inhibitor may be contained within a composition that may, for example, be administered by injection into the blood stream.

Injections may be intravenous (bolus or infusion), subcutaneous, intramuscular or a direct injection into the target tissue (e.g. an intraventricular injection-when used in the brain). The inhibitors may also be administered by inhalation (e.g. intranasally) or even orally (if appropriate).

The inhibitors of the invention may also be incorporated within a slow or delayed release device. Such devices may, for example, be inserted in the body of the individual, and the molecule may be released over weeks or months. Such devices may be particularly advantageous when long term treatment with an inhibitor is required and which would normally require frequent administration (e.g. at least daily injection).

It will be appreciated that the amount of an inhibitor that is required is determined by its biological activity and bioavailability which in turn depends on the mode of administration, the physicochemical properties of the molecule employed and whether it is being used as a monotherapy or in a combined therapy. The frequency of administration will also be influenced by the above-mentioned factors and particularly the half-life of the inhibitor within the individual being treated.

Optimal dosages to be administered may be determined by those skilled in the art, and will vary with the particular inhibitor in use, the strength of the preparation, and the mode of administration.

Additional factors depending on the particular individual being treated will result in a need to adjust dosages, including subject age, weight, gender, diet, and time of administration.

For example, pimozide is administered at an initial dose of 1 to 2 mg orally per day in divided doses. The maintenance dose is typically less than 0.2 mg/kg or 10 mg/day, whichever is less. The maximum dose is 10 mg/day.

In a further example, Penfluridol is administered orally at an initial dose of 20-60 mg/week, up to a maximum of 250 mg/week.

In one embodiment, in addition to the calcium channel inhibitor, the individual is also administered one or more further therapeutic agents for treating an amyloid disease of the nervous system and/or a neurodegenerative disease and/or condition in an individual. The further therapeutic agent may be a hormone, a small molecule, a polypeptide, a peptide, an antibody, a polynucleotide, a peptidomimetic, or a natural product.

In a fourth aspect, the invention provides a pharmaceutical composition comprising a calcium channel inhibitor of the invention, and a pharmaceutically acceptable diluent, carrier or excipient.

By “pharmaceutically acceptable” we include the meaning that the formulation is sterile and pyrogen free. Suitable pharmaceutical carriers, diluents and excipients are well known in the art of pharmacy. The carrier(s) must be “acceptable” in the sense of being compatible with the inhibitor and not deleterious to the recipients thereof. Typically, the carrier(s) includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof which will be sterile and pyrogen free. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the pharmacological agent.

In an embodiment, the pharmaceutical compositions or formulations of the invention are for parenteral administration, more particularly for intravenous administration. In a preferred embodiment, the pharmaceutical composition is suitable for intravenous administration to a patient, for example by injection.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

In an alternative preferred embodiment, the pharmaceutical composition is suitable for topical administration to a patient.

Preferably, the formulation is a unit dosage containing a daily dose or unit, daily sub-dose or an appropriate fraction thereof, of the active ingredient.

The inhibitor or active ingredient may be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the inhibitor or active ingredient will generally be administered in admixture with a suitable pharmaceutical excipient, diluent or carrier selected with regard to the intended route of administration and standard pharmaceutical practice.

For example, the inhibitor or active ingredient may be administered orally, buccally or sublingually in the form of tablets, capsules, ovules, elixirs, solutions or suspensions, which may contain flavouring or colouring agents, for immediate-, delayed- or controlled-release applications. The active ingredient may also be administered via intracavernosal injection.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The inhibitor or active ingredient can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of an agent, antibody or compound will usually be from 1 to 1,000 mg per adult (i.e. from about 0.015 to 15 mg/kg), administered in single or divided doses.

Thus, for example, the tablets or capsules of the agent or active ingredient may contain from 1 mg to 1,000 mg of agent or active agent for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The inhibitor or active ingredient can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A or 1,1,1,2,3,3,3-heptafluoropropane (HFA 227EA), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an active ingredient and a suitable powder base such as lactose or starch. Such formulations may be particularly useful for treating solid tumours of the lung, such as, for example, small cell lung carcinoma, non-small cell lung carcinoma, pleuropulmonary blastoma or carcinoid tumour.

Aerosol or dry powder formulations are preferably arranged so that each metered dose or “puff” contains at least 1 mg of the inhibitor for delivery to the patient. It will be appreciated that the overall daily dose with an aerosol will vary from patient to patient, and may be administered in a single dose or, more usually, in divided doses throughout the day.

Alternatively, the inhibitor or active ingredient can be administered in the form of a suppository or pessary, particularly for treating or targeting colon, rectal or prostate tumours.

The inhibitor or active ingredient may also be administered by the ocular route. For ophthalmic use, the inhibitor can be formulated as, e.g., micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum. Such formulations may be particularly useful for treating solid tumours of the eye, such as retinoblastoma, medulloepithelioma, uveal melanoma, rhabdomyosarcoma, intraocular lymphoma, or orbital lymphoma.

The inhibitor or active ingredient may be applied topically in the form of a lotion, solution, cream, ointment or dusting powder, or may be transdermally administered, for example, by the use of a skin patch. For application topically to the skin, the active ingredient can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. Such formulations may be particularly useful for treating solid tumours of the skin, such as, for example, basal cell cancer, squamous cell cancer or melanoma.

Formulations suitable for topical administration in the mouth include lozenges comprising the active ingredient in a flavoured basis, usually sucrose and acacia or tragacanth; pastilles comprising the agent or active ingredient in an inert basis such as gelatin and glycerin, or sucrose and acacia; and mouth-washes comprising the active ingredient in a suitable liquid carrier. Such formulations may be particularly useful for treating solid tumours of the mouth and throat.

In an embodiment, the inhibitor or active ingredient may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The agent or inhibitor ingredient can be administered by a surgically implanted device that releases the drug directly to the required site, for example, into the eye to treat ocular tumours. Such direct application to the site of disease achieves effective therapy without significant systemic side-effects.

An alternative method for delivery of the inhibitor or active ingredients is the Regel injectable system that is thermo-sensitive. Below body temperature, Regel is an injectable liquid while at body temperature it immediately forms a gel reservoir that slowly erodes and dissolves into known, safe, biodegradable polymers. The active drug is delivered over time as the biopolymers dissolve.

Polypeptide pharmaceuticals can also be delivered orally. The process employs a natural process for oral uptake of vitamin B12 in the body to co-deliver proteins and peptides. By riding the vitamin B12 uptake system, the protein or peptide can move through the intestinal wall. Complexes are synthesised between vitamin B12 analogues and the drug that retain both significant affinity for intrinsic factor (IF) in the vitamin B12 portion of'the complex and significant bioactivity of the drug portion of the complex.

Polynucleotides may be administered as a suitable genetic construct as described below and delivered to the patient where it is expressed. Typically, the polynucleotide in the genetic construct, is operatively linked to a promoter which can express the compound in the cell. The genetic constructs of the invention can be prepared using methods well known in the art, for example in Sambrook et al (2001).

Although genetic constructs for delivery of polynucleotides can be DNA or RNA, it is preferred if they are DNA.

Preferably, the genetic construct is adapted for delivery to a human cell. Means and methods of introducing a genetic construct into a cell are known in the art, and include the use of immunoliposomes, liposomes, viral vectors (including vaccinia, modified vaccinia, lentivurus, parvovirus, retroviruses, adenovirus and adeno-associated viral (AAV) vectors), and by direct delivery of DNA, e.g. using a gene-gun and electroporation. Furthermore, methods of delivering polynucleotides to a target tissue of a patient for treatment are also well known in the art. In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

The inhibitor or active ingredients of the invention (i.e. a calcium channel inhibitor for use) may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of protein activity loss and that use levels may have to be adjusted upward to compensate. In one embodiment, the lyophilised (freeze dried) active ingredient loses no more than about 20%, or no more than about 25%, or no more than about 30%, or no more than about 35%, or no more than about 40%, or no more than about 45%, or no more than about 50% of its activity (prior to lyophilisation) when re-hydrated.

Preferably, the pharmaceutical composition further comprises one or more therapeutic agent for treating and/or preventing an amyloid disease of the nervous system in an individual.

It is therefore appreciated that although the inhibitors of the invention described above may be clinically effective in the absence of any other therapeutic agent, it may be advantageous to administer the inhibitor of the invention in conjunction with a further therapeutic agent (e.g. anti-neurodegenerative agent).

By “anti-neurodegenerative agent” we include a therapeutic and/or prophylactic agent which can be a drug or other agent used to treat and/or prevent an amyloid disease and/or neurodegenerative disease as described herein. Any of the anti-neurodegenerative agents listed below, or any other such agent known or discovered to exhibit a therapeutic or diagnostic effect in the diseases described herein, may be formulated into a pharmaceutical composition as described.

Anti-neurodegenerative agents include, for example, anticholinergics, dopamine precursors (e.g., L-dopa (Sinemet, carbidopa)), COMT inhibitors, dopamine receptor agonists, MAO-B inhibitors, bromocriptine (Parlodel), pergolide (Permax), benztropine (Cogentin), amantadine (Symmetrel), trihexyphenidyl (Artane) and deprenyl (Eldepryl, selegiline), Huperzine A, acetylcholinesterase (AChE) inhibitors, N-methyl-D-aspartate (NMDA) receptor antagonists (e.g., Namenda (Memantine)), and cholinesterase inhibitors (e.g., Aricept (donepezil), Reminyl (Galantamine), Exelon (rivastigmine), Cognex (Tacrine).

Preferably, the pharmaceutical composition according to the fourth aspect of the invention is for use in treating and/or preventing an amyloid disease of the nervous system in an individual.

The invention also provides use of pharmaceutical composition according to the invention in the manufacture of a medicament for treating and/or preventing an amyloid disease of the nervous system in an individual.

The invention also provides a method for treating and/or preventing an amyloid disease of the nervous system in an individual comprising administering a pharmaceutical composition according to the invention to an individual in need thereof.

The “amyloid disease of the nervous system” is as defined herein.

In an embodiment of the invention, the medicament containing the inhibitor of the invention may also comprise at least one further therapeutic agent (e.g. anti-neurodegenerative agent).

In an embodiment of the methods of the invention, the method may also comprise administering to the individual at least one further therapeutic agent (e.g. an anti-neurodegenerative agent). The method may comprise administering to the individual a pharmaceutical composition containing the inhibitor of the invention (e.g. calcium channel inhibitor), and the further therapeutic agent (e.g. anti-neurodegenerative agent). However, it is appreciated that the inhibitor of the invention and further therapeutic agent (e.g. anti-neurodegenerative agent) may be administered separately, for instance by separate routes of administration. Thus, it is appreciated that the inhibitor and the at least one further therapeutic agent can be administered sequentially or (substantially) simultaneously. They may be administered within the same pharmaceutical formulation or medicament or they may be formulated and administered separately.

It will also be appreciated that the invention also provides a method of treatment, wherein a further therapeutic agent (e.g. anti-neurodegenerative agent) is administered to an individual in need thereof, wherein the individual is one who is administered an inhibitor of the invention. The administration of the further therapeutic agent and inhibitor of the invention may occur at the same time, although the individual may have been (or will be) administered the inhibitor of the invention before (or after) receiving the medicament containing the further therapeutic agent (e.g. anti-neurodegenerative agent).

When the further anti-neurodegenerative agent has been shown to be particularly effective for a specific neurodegenerative disease, it may be preferred that the inhibitor of the invention is used in combination with that further anti-neurodegenerative agent to treat that specific neurodegenerative disease.

In a fifth aspect, the invention provides a kit of parts comprising: (i) an inhibitor according to first, second, third or fourth aspect of the invention; and/or (ii) a pharmaceutically acceptable diluent, carrier or excipient; and/or (ii) at least one additional therapeutic agent.

Preferably, the kit according to the invention is for use in treating and/or preventing an amyloid disease of the nervous system and/or a neurodegenerative disease and/or condition in an individual, as defined herein.

The invention also provides use of a kit according to the fifth aspect of the invention in the manufacture of a medicament for treating and/or preventing an amyloid disease of the nervous system in an individual.

The invention also provides a method for treating and/or preventing an amyloid disease of the nervous system in an individual comprising administering a kit according to the fifth aspect of the invention to an individual in need thereof.

In a sixth aspect, the invention provides a method for identifying an agent for treating and/or preventing an amyloid disease of the nervous system in an individual, the method comprising the steps of:

(i) providing a candidate calcium channel inhibitor to be tested; and

(ii) testing the candidate inhibitor in a model of neurodegenerative disease.

Preferably, the method further comprises the step of testing whether the candidate calcium channel inhibitor is capable of increasing proteolysis and/or activating the proteasome.

Preferably, the method further comprises the step of testing whether the candidate calcium channel inhibitor is capable of reducing protein aggregation and/or protein misfolding.

Preferably, the method further comprises the step of testing whether the candidate calcium channel inhibitor is capable of reducing the number and/or size of amyloid aggregates in the nervous system of the individual.

Methods of measuring proteolysis and/or activating the proteasome are known in the art and are described herein.

In an embodiment of the method of the sixth aspect of the invention, a candidate inhibitor that provides cognitive gain of function in a neurodegenerative disease model may be identified as an agent that is useful in the treatment of an amyloid disease of the nervous system and/or a neurodegenerative disease and/or condition in an individual.

The methods of the sixth aspect of he invention, may be carried out in vitro, in vivo or ex vivo.

In an embodiment, the model of a neurodegenerative disease allows the identification of functional deficits in neurons. In an embodiment, the model of neurodegenerative disease is a Drosophila, neuronal cells derived from induced pluripotent stem (iPS) cells, or a mouse model. In an embodiment, the model of neurodegenerative disease is a model of Alzheimer's disease.

In an embodiment the model system is a Drosophila fly model overexpressing Aβ.

In another embodiment, the model system is neuronal cells derived from iPS cells from an individual with a neurodegenerative disease, such as Alzheimer's disease. For example, skin fibroblasts from an individual with familial Alzheimer's disease may be reprogrammed to iPS cells, then to neuroepithelial stem (NES) cells as previously described (Falk et al PLoS One 7, e29597 (2012)). Reprogrammed skin fibroblasts from a healthy individual may be used as a control.

In still another embodiment, the model system is a mouse model of neurodegenerative disease such as Alzheimer's disease. In a further embodiment, the model system is a mouse model of AD. In an embodiment, the mouse model is established by the introduction of recombinant Aβ1-42 into the wild-type mouse brain. This provides an in vivo model which replicates the neuronal death aspect of amyloid diseases. In a preferred embodiment, the mouse model is transgenic and overexpress proteins linked to familial AD (FAD), mutant amyloid precursor protein (APP), or APP and presenilin (PS). For example, the 5XFAD transgenic mice which carries five FAD mutations in APP and PS1 transgenes (APPK670N/M671L/I716V/V717I Tg and PSEN1M146L/L286V Tg) driven by the Thy-1 promoter could be used. These mice exhibit neuron loss and memory deficits that are associated with amyloid pathology (Oakley H, Cole S L, Logan S, Maus E, Shao P, Craft J, Guillozet-Bongaarts A, Ohno M, Disterhoft J, Van Eldik L, Berry R, Vassar R. J Neurosci. 2006 Oct. 4; 26(40)10129-40). It will be appreciated by those skilled in the art that the App knock-in mouse model could also be used (Saito T, Matsuba Y, Mihira N, Takano J, Nilsson P, Itohara S, Iwata N, Saido T C. Nat Neurosci. 2014 May; 17(5):661-3).

In an embodiment, of part (ii) of the sixth aspect of the invention, the test is an electrophysiological, morphological and/or behavioural test. In an embodiment, part (ii) of the sixth aspect of the invention further comprises, measuring in the model of neurogenerative disease at least one parameter selected from the group comprising: resting membrane potential; action potentials; gamma and/or theta oscillations; the frequency and/or size of amyloid deposits; and/or cognitive function. Cognitive function may be assessed using behavioural tests selected from the group comprising: negative geotaxis climbing assay; Morris water maze; and the Y-maze.

By “resting membrane potential” we include the meaning of the relatively static membrane potential of quiescent cells, also referred to as “passive membrane potential”. These two terms are used interchangeably herein.

In an embodiment the electrophysiological test is one or more from the group comprising: the assessment of passive/resting membrane properties; measurement of gamma and/or theta oscillations. In an embodiment, the morphological test is one or more from the group comprising: analysis of Aβ histology (plaque size and frequency); analysis of neuronal morphology, such as astrocyte number). In an embodiment the behavioural test is one or more from the group comprising: negative geotaxis climbing assay; Morris water maze; and the Y-maze. Such test are well known in the art.

For example, an assay for identifying a candidate calcium channel inhibitor that provides a gain of function in the Drosophila model may be performed as follows. Aβ overexpressing, and control Drosophila strains, may be treated with the candidate inhibitor. Behavioural testing, such as the negative geotaxis climbing assay, may be performed. The results may be analysed to evaluate whether the candidate inhibitor provided a gain of function in the Aβ overexpressing Drosophila.

In a further example, an assay for identifying a candidate calcium channel inhibitor that provides a gain of function in a Drosophila model may be performed as follows. Aβ overexpressing, and control Drosophila strains may be treated with the candidate inhibitor. Histology may be carried out on the Drosophila brain to evaluate the effect on plaque burden of the candidate inhibitor. The histology of the Drosophila brain may be carried out using techniques known in the art such as whole-mount immunohistochemistry followed by confocal microscopy.

As can be seen from Example 1, the inventors treated an Aβ overexpressing Drosophila model and control strain with one of the calcium channel inhibitors (pimozide) and evaluated the outcome with behavioural testing, using the negative geotaxis climbing assay, as well as Aβ histology (plaque burden). In the behavioural test a clear functional improvement could be seen of the treated animals over the sham treated flies, while the control strains given the drug showed no adverse effect (FIG. 3B). The inventors then evaluated the calcium channel inhibitor treatment by assessment of Aβ histology (plaque burden). After dissecting out the Drosophila brains and straining for Aβ, a clear reduction of the number as well as size of Aβ aggregates was seen in the pimozide-treated group of flies (FIG. 3C-E). This suggests that the calcium channel inhibitor provides cognitive gain of function in the Drosophila neurodegenerative disease model.

In a further example, an assay for identifying a candidate calcium channel inhibitor that provides a gain of function in the IPS model may be performed as follows. An iPS cell-derived neuronal culture from a patient with and without a neurodegenerative disease, such as Alzheimer's disease may be established. Control and diseased cells may be treated with the candidate inhibitor and electrophysiological parameters, such as passive membrane properties may be measured. Morphological characterization of human neurons from healthy wildtype and Alzheimer's disease patients may also be carried out. The morphological characterization may be carried out using techniques known in the art, such as brightfield microscopy, immunohistochemistry, immunofluorescence and confocal microscopy.

It will be appreciated that the inhibitor and assays of the invention lend themselves to personalised medicine in the clinic whereby the most appropriate inhibitor to be administered to the patient is determined, and either selected or prepared in the clinic. By using an iPS cell-derived neuronal culture from a patient with a neurodegenerative disease, candidate calcium channel inhibitors can be tested on the cells from that patient.

In this way, it will be possible to identify candidate inhibitors that will specifically work in an individual.

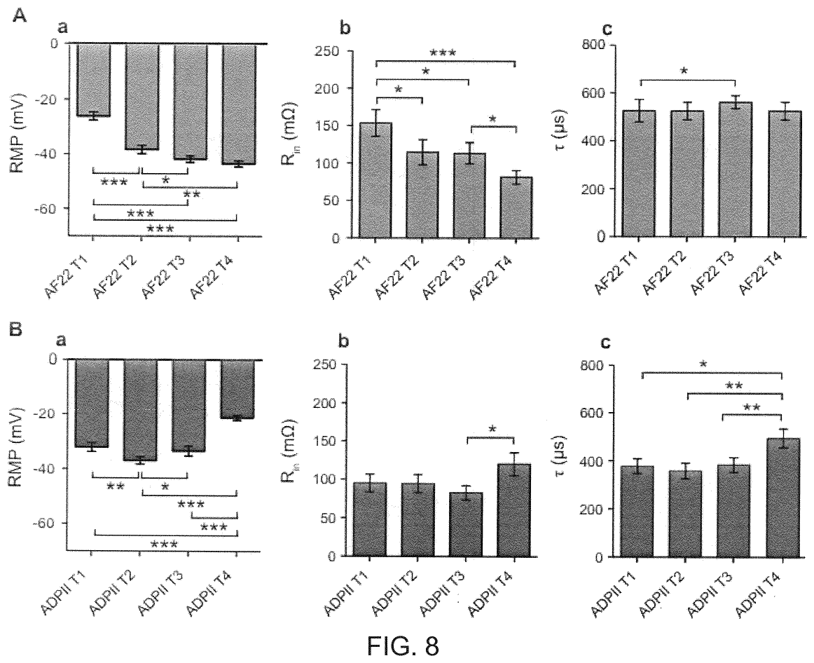

As can be seen from Example 1, the inventors established iPS cell-derived neuronal cultures in the laboratory and performed electrophysiological assays. The patient-derived AD neurons exhibited a lower resting membrane potential and a reduced ability to fire action potentials. After treating the cells with two different T-type calcium channel inhibitors (pimozide, penfluridol), both electrophysiological parameters were normalised to healthy control levels. The healthy patient control cultures when treated showed no effect from the drugs itself (FIGS. 8, 9, 10).

An assay for identifying a candidate calcium channel inhibitor that provides a gain of function in a mouse model of neurodegenerative disease may be performed as follows. A mouse model of neurodegenerative disease, such as those described above may be established. The mouse may be treated with a candidate inhibitor and electrophysiological parameters measured. For example, a patch clamp assay may be performed, or network rhythms such as gamma oscillations can be measured in mouse hippocampal slices. Gamma and/or theta oscillations may be measured by methods known in the art, such as EEG or MEG. Morphological characterization and the quantitation of plaque burden in wildtype and diseased mice may be performed. The morphological characterization may be carried out using techniques known in the art, such as brightfield microscopy, immunohistochemistry, immunofluorescence and confocal microscopy. Plaque burden may be quantitated. Behavioural testing may be performed on wildtype and diseased mice. Behavioural testing may include the Morris water maze and/or the Y-maze.

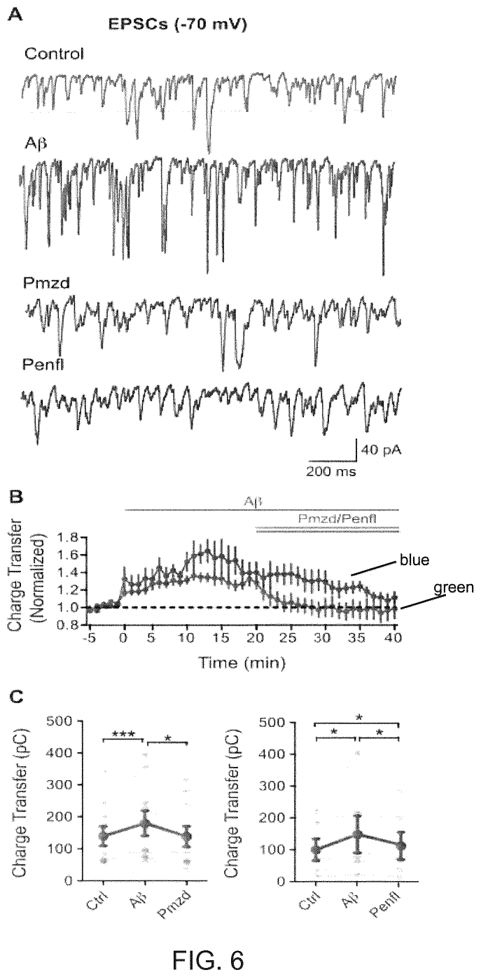

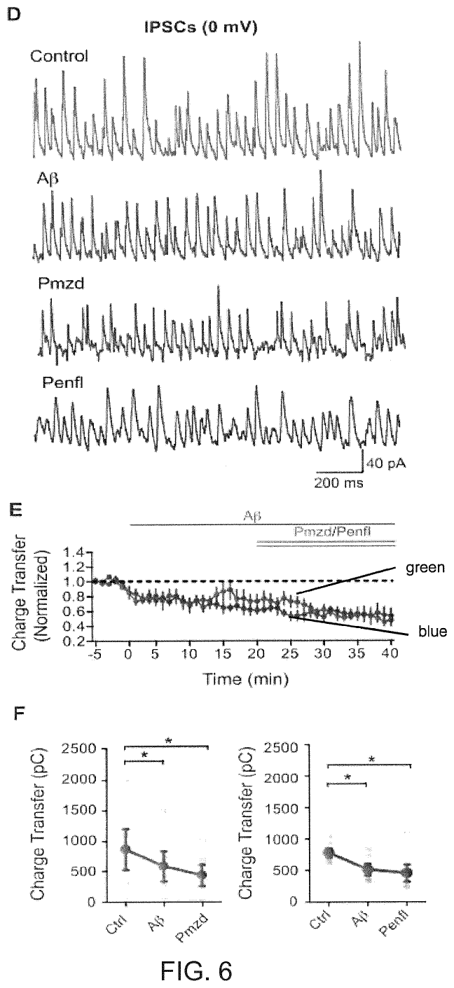

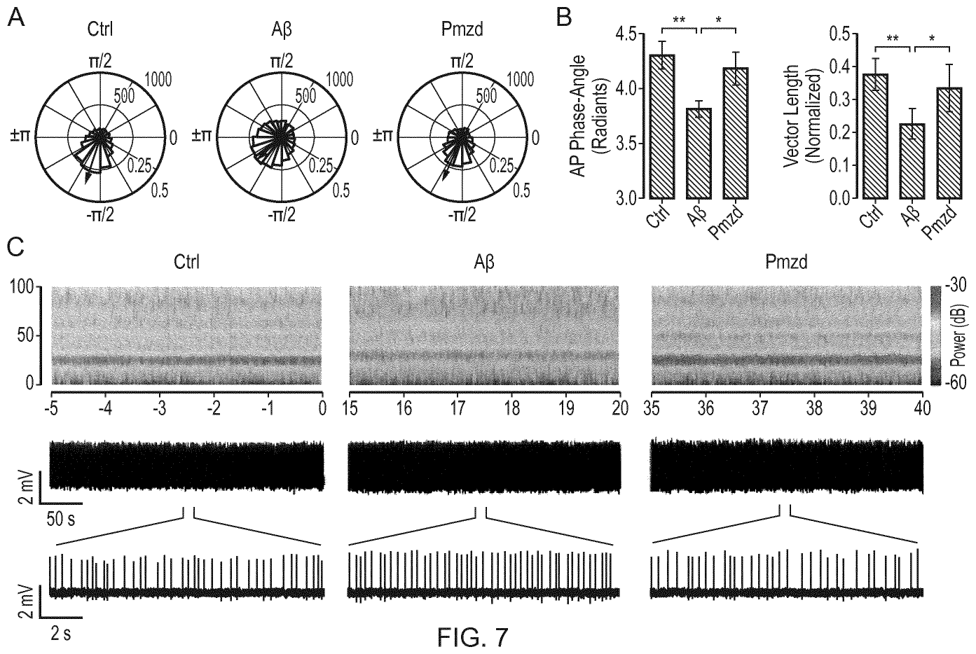

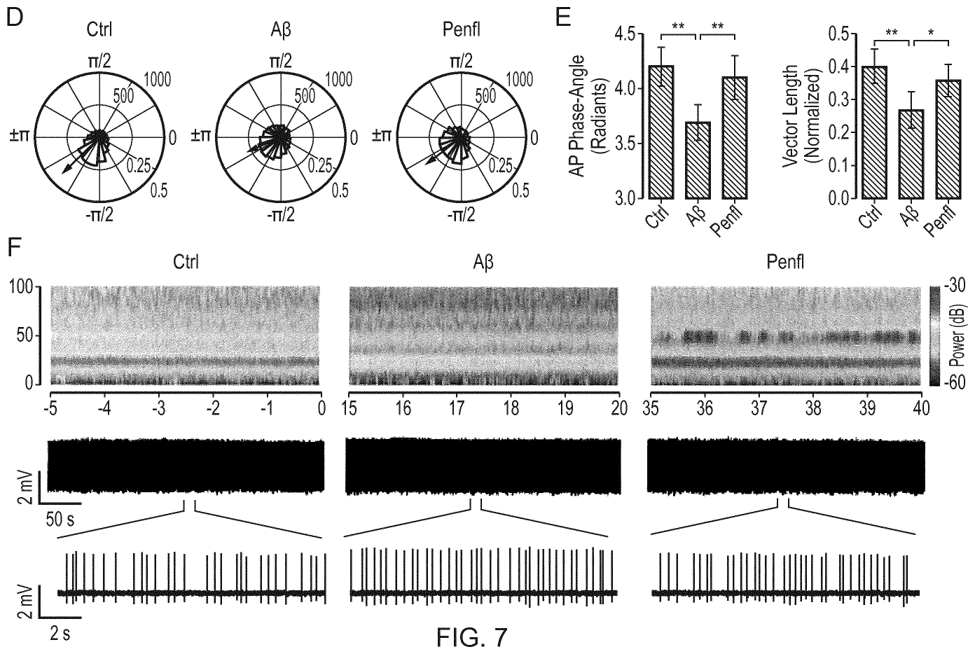

As can be seen from Example 1, the inventors demonstrated that two calcium channel inhibitors (pimozide and penfluridol) are able to prevent the Aβ-induced degradation of gamma oscillations observed in local field recordings in the mouse hippocampal network (FIG. 4). It was also demonstrated using pimozide and penfluridol that T-type calcium channel inhibitors also rescue the shift in excitatory/inhibitory balance in the neuronal network (FIG. 6) and action potential desynchronization caused by Aβ (FIG. 7).

It is appreciated that the screening methods can be used to identify agents that may be useful in preventing and/or treating an amyloid disease of the nervous system and/or a neurodegenerative disease and/or condition in an individual. Thus, the screening methods preferably also comprise the further step of testing the identified compound or the modified compound for efficacy in an animal model of a neurodegenerative disease. Suitable models are described above. The invention may comprise the further step of synthesising an/or purifying the identified compound or the modified compound. The invention may further comprise the step of formulating the compound into a pharmaceutically acceptable composition. Compounds may also be subjected to other tests, for example toxicology or metabolism tests, as is well known to those skilled in the art.

All of the documents referred to herein are incorporated herein, in their entirety, by reference. The listing or discussion of an apparently prior published document in this specification should not necessarily be taken as an acknowledgement that the document is part of the state of the art or is common general knowledge.

Preferred, non-limiting examples which embody certain aspects of the invention will now be described, with reference to the following figures:

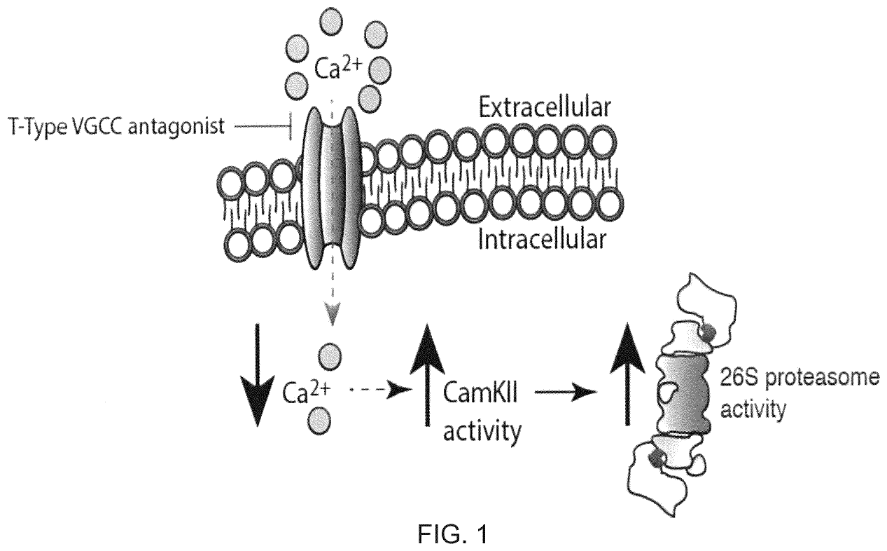

FIG. 1: Calcium-driven mechanisms underlying proteasome activation

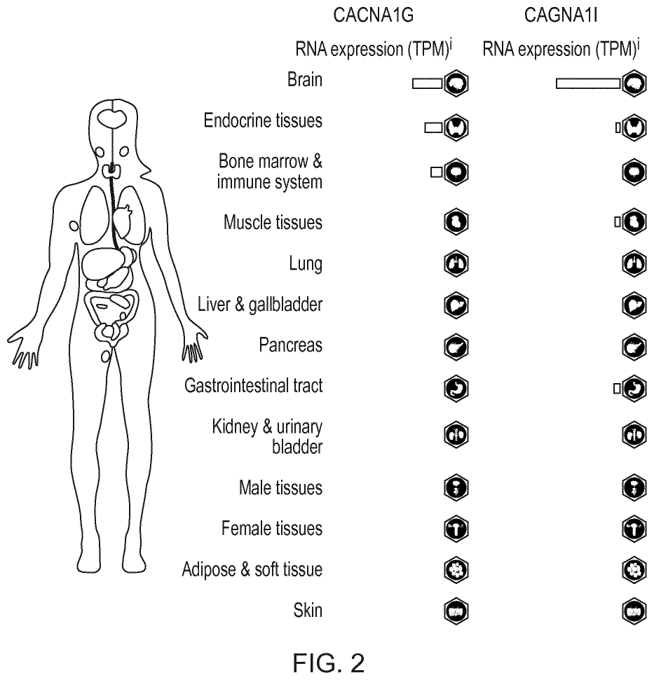

FIG. 2: Tissue distribution of T-type calcium channels

FIG. 3: Effects of pimozide on behaviour and Aβ aggregates in a Drosophila model of Alzheimer's disease. A. Wild Type flies (Arc2E and Elav) and Experimental flies (ElavArc2E) were tested for their ability to climb at day 15 after hatching. Pimozide improves the climbing test performance of the flies overexpressing the experimental flies (ElavArc2E+Pmzd) compared to the non-treated flies. B. Climbing ability over time. Flies treated with pimozide could climb significantly better than the non-treated for 25 days after hatching, while their climbing ability degenerated at 35 days of age. C. Mushroom bodies of 35 days old Drosophila with Aβ aggregates. On panels a and d (top) neurons expressing GFP (green), in panels b and e (middle) Amyloid-β aggregates, on panels c and f (bottom) merge of the previous two IF images. Panels on the left side are representative of mutated Drosophila (ElavArc2E) fed with food containing only vehicle, panels on the right mutated Drosophila (ElavArc2E+Pmzd) fed with food containing 5 μM pimozide, showing significantly less Aβ aggregates. D. Quantification of the aggregates size of mutated flies non-treated (red) or treated (green): flies treated with pimozide show a significant reduction in the aggregates size. E. Quantification of the number of aggregates in mutated flies non treated (red) or treated (green): treated flies show a significant reduction in the number of aggregates per μm². Data is presented as mean±SEM. *** indicates p<0.001.

FIG. 4: Inhibition of T-type Ca²⁺ channels prevents Aβ toxicity in mice hippocampal slices. A. Representative sample traces of kainate-induced gamma oscillations in hippocampal slices under control condition (100 nM KA, grey), after 15 min incubation with 50 nM Aβ (red) and after 1 h incubation with pimozide 1 μM (light green), pimozide 10 μM (dark green) or penfluridol 5 μM (blue), in the last 15 min of which Aβ (50 nM) was added. B. Summary bar graph of the integrated gamma power of several compounds tested, some with no effect in preventing the damage on gamma oscillations produced by Aβ; SB269970 (5-HT receptor antagonist, light blue); sulpiride (D₂, D₃ receptor antagonist, liliac); L745,870 (D₄ receptor antagonist, yellow); others able to rescue to effect of Aβ: pimozide (T-type Ca²⁺ channel blocker, two different concentration, green); penfluridol (T-type Ca²⁺ channel blocker, blue). C. Mean integrated power spectra of gamma oscillations for each condition: control (grey), Aβ (red), pimozide 1 μM (light green) pimozide 10 μM (dark green) and penfluridol 5 μM (blue). D. Quality of gamma oscillation (coefficient of rythmicity; Cr) reduction by Aβ and T-type Ca²⁺ channel blockers' protective effect with summary bar graph (E) of Cr from the experimental condition described in A and C. F. Summary bar graph of the integrated gamma power under control condition (grey), Aβ (red), two different concentrations of pimozide (green) and penfluridol (blue). Data is presented as mean±SEM. * indicates p<0.05 and *** p<0.001, compared to control. # indicates p<0.05 and ### p<0.001, compared to Aβ.

FIG. 5: Rescue of Aβ toxicity by different types of T-type Ca²⁺ channel inhibitors in mice hippocampal slices. A. Representative traces of gamma oscillation in hippocampal slices under control condition (grey), after 1 μM Aβ application (red), after 10 μM pimozide (green) or 5 μM penfluridol (blue). B. Integrated power of gamma oscillations (20-80 Hz) from the experimental conditions described in A. C. Summary bar graph of gamma oscillation power under control condition (grey), Aβ (red), pimozide (green), penfluridol (blue) and other two structurally different T-type Ca²⁺ channel blockers: 10 μM NNC55-0396 (pink) and 5 μM ML-218 (light blue). D. Time course and design of rescue experiments. Slices were treated for 20 min with KA 100 nM to induce oscillations (last 5 min represented here), 20 min application of Aβ followed by 30 min application of either pimozide or penfluridol (still in presence of Aβ 1 μM). □Both pimozide and penfluridol partially rescue gamma oscillation power (quantification represented in C). Data is presented as mean±SEM. * indicates p<0.05.

FIG. 6: T-type Ca²⁺ channel inhibition rescues Aβ-induced impairment of EPSCs but not IPSCs. A. Representative traces of EPSCs in control condition (grey), after Aβ application (red) and after either pimozide (green) or penfluridol (blue) application in slices activated with 100 nM KA. B. Time courses of the charge transfer of EPSCs. Slices were treated for 20 min with KA 100 nM (last 5 min represented here), 20 min application of Aβ 1 μM followed by 20 min application of either pimozide 10 μM (green) or penfluridol 5 μM (blue) (still in presence of Aβ 1 μM). C. Summary graph of charge transfer of EPSCs showing each experiment and their mean. D. Representative traces of IPSCs in control condition (grey), after Aβ application (red) and after either pimozide (green) or penfluridol (blue) application in slices activated with 100 nM KA. E. Time courses of the charge transfer of IPSCs. Slices were treated for 20 min with KA 100 nM (last 5 min represented here), 20 min application of Aβ 1 μM followed by 20 min application of either pimozide 10 μM (green) or penfluridol 5 μM (blue) (still in presence of Aβ 1 μM). F. Summary graph of charge transfer of IPSCs showing each experiment and their mean. Data is presented as mean±SEM. * indicates p<0.05 and *** p<0.001.

FIG. 7: T-type Ca²⁺ channel inhibition rescues the Aβ-induced desynchronization of AP firing. A. Polar-plots showing the distribution of AP phase-angles in control condition (grey), after Aβ application (red) and after pimozide application (green). Phase-angles and gamma oscillation-phases are represented in radians. The peak of the oscillation cycle corresponds to 0π and the valley corresponds to ±π. B. Summary bar graph of AP phase-angle and vector length (normalized) in control condition (grey), after Aβ application (red) and after pimozide application (green). C. Representative LFP spectrograms and corresponding single unit recordings (2 different magnification) in control condition, after Aβ□application and after pimozide application. The power of the oscillation is significantly reduced during Aβ application and restored after 20 min pimozide application. The AP firing rate is significantly increased by Aβ and rescued by pimozide. D. Polar-plots showing the distribution of AP phase-angles in control condition (grey), after Aβ application (red) and after penfluridol application (blue). E. Summary bar graph of AP Phase-angle and vector length (normalized) in control condition (grey), after Aβ application (red) and after penfluridol application (blue). F. Representative LFP spectrograms and corresponding single unit recordings (2 different magnification) in control condition, after Aβ□application and after penfluridol application. The power of the oscillation is significantly reduced during Aβ application and restored after 20 min penfluridol application. The AP firing rate is significantly increased by Aβ and rescued by penfluridol. Data is presented as mean±SEM. * indicates p<0.05 and ** p<0.01.

FIG. 8: Evolution of passive membrane properties over time of two lines of iPSC-derived neurons. A. AF22 control cell line (from healthy human patients, grey). a. The average resting membrane potential (RMP in mV) decreases over time. b. The membrane input resistance (Rᵢₙ in MΩ) decreased over time. c. The membrane time constant (τ in □s) changes in an unpredictable manner. B. ADPII cell-line (from AD patients, red). a. The average resting membrane potential decreases until day 55 and then degenerates over time. b. The membrane input resistance does not change until day 65 and then increases drastically. c. The membrane time constant does not change until day 65 and then increases drastically. T1 (36-45 days after differentiation), T2 (46-55 days after differentiation), T3 (56-65 days after differentiation), T4 (66-75 days after differentiation). Data is presented as mean±SEM. * indicates p<0.05, ** p<0.01 and *** p<0.001.

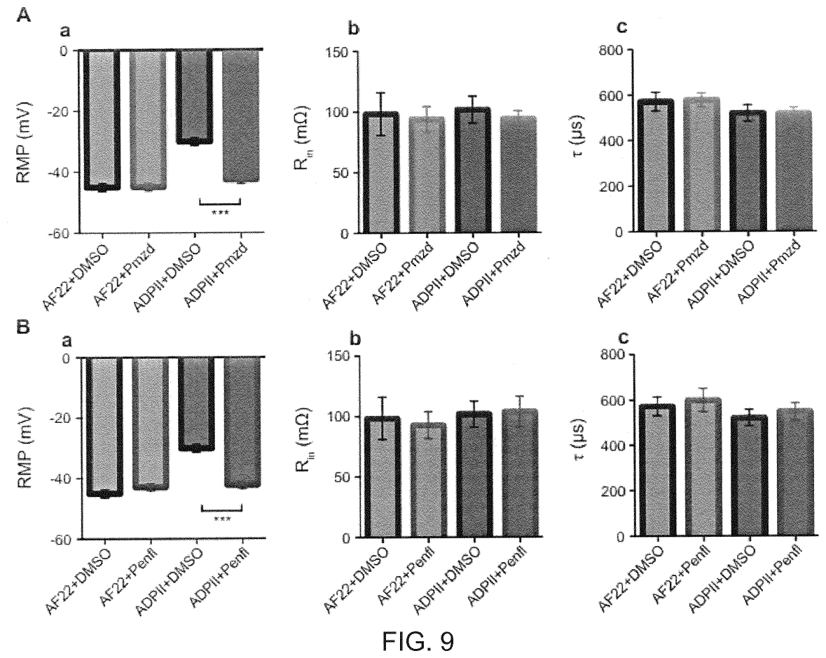

FIG. 9: Passive membrane properties changes in cells treated with pimozide or penfluridol at T3 (56-65 days after differentiation). A. AF22 and ADPII iPS cells treated for 48 hours with either just vehicle (DMSO) or pimozide 1 μM. a. Resting membrane potential does not change in AF22 cells in presence of DMSO (grey and black) or pimozide (grey and green). It drastically changes in ADPII cells bringing the resting membrane potential back to control levels after 48 hours treatment with pimozide (red and green). b. No significant changes in membrane input resistance in any condition. c. No significant changes in membrane time constant in any condition. B. AF22 and ADPII iPS cells treated for 48 hours with either just vehicle (DMSO) or penfluridol 1 μM. a. Resting membrane potential does not change in AF22 cells in presence of DMSO (grey and black) or penfluridol (grey and blue). It drastically changes in ADPII cells bringing the resting membrane potential back to control levels after 48 hours treatment with penfluridol (red and blue). b. No significant changes in membrane input resistance in any condition. c. No significant changes in membrane time constant in any condition. Data is presented as mean±SEM. *** indicates p<0.001.

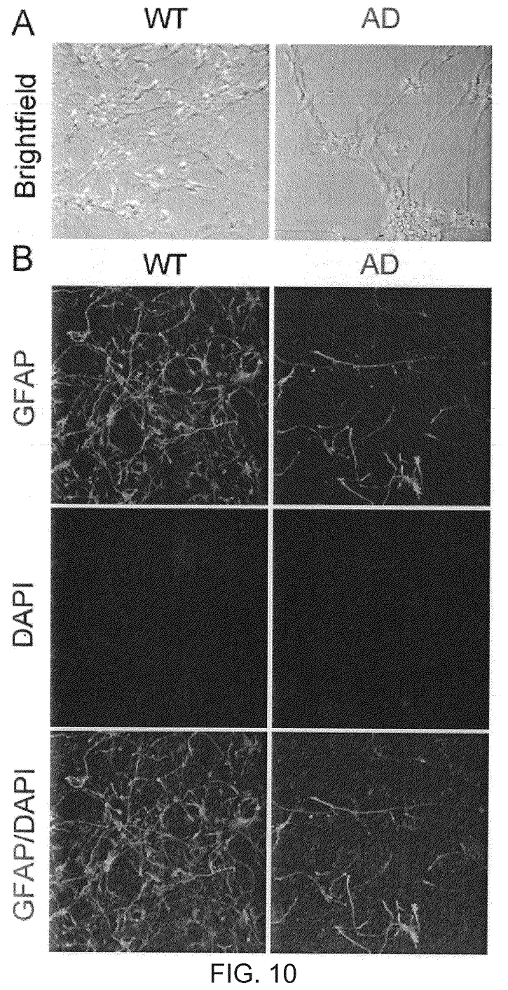

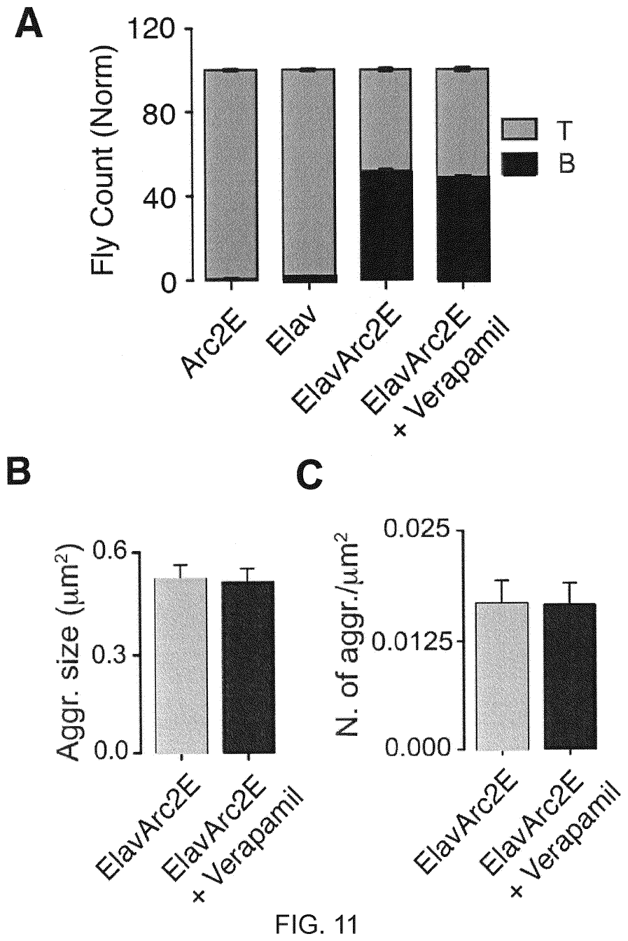

FIG. 10: Morphological characterization of human neurons from healthy wildtype and Alzheimer's disease patients. A. Brightheld images of differentiated iPS cell-derived neurons from wildtype (AF22, left) and Alzheimer's disease (ADPII, right) patients at the T3 time point when AD neurons are severely degenerating. B. Immunohistochemistry in degenerating neurons reveals significantly fewer astrocytes (GFAP, top green panels) and total number of cells (DAPI, middle blue panels) in the AD patient line compared to wildtype. The merge of GFAP and DAPI shows that there are many more DAPI labelled neurons that are negative for GFAP (GFAP/DAPI, bottom panels). This demonstrates that other neuronal cell types are lost in Alzheimer's disease iPS cells.

FIG. 11: Effects of the L-type Ca²⁺ channel inhibitor verapamil on behavior and Aβ aggregates in a Drosophila model of Alzheimer's disease. A. Wild Type flies (Arc2E and Elav) and Experimental flies (ElavArc2E) were tested for their ability to climb at day 15 after hatching. Verapamil failed to improve the climbing test performance of the flies overexpressing the experimental flies (ElavArc2E+Verapamil) compared to the non-treated flies. B. Quantification of the aggregates size of mutated flies non-treated or treated: flies treated with verapamil showed no reduction in the aggregates size. C. Quantification of the number of aggregates in mutated flies non treated or treated: treated flies showed no reduction in the number of aggregates per μm². Data is presented as mean±SEM. *** indicates p<0.001.

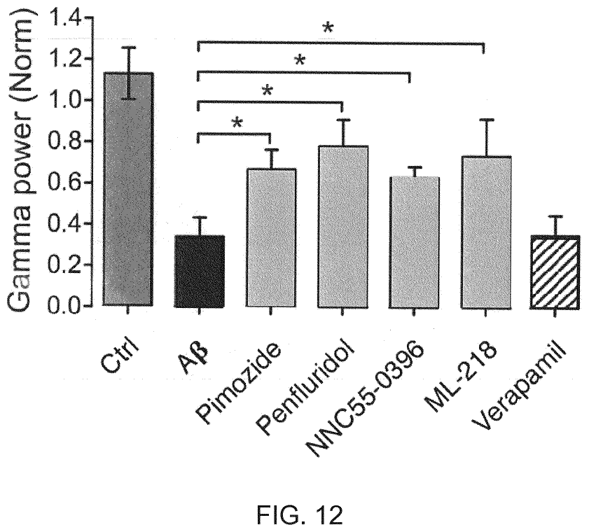

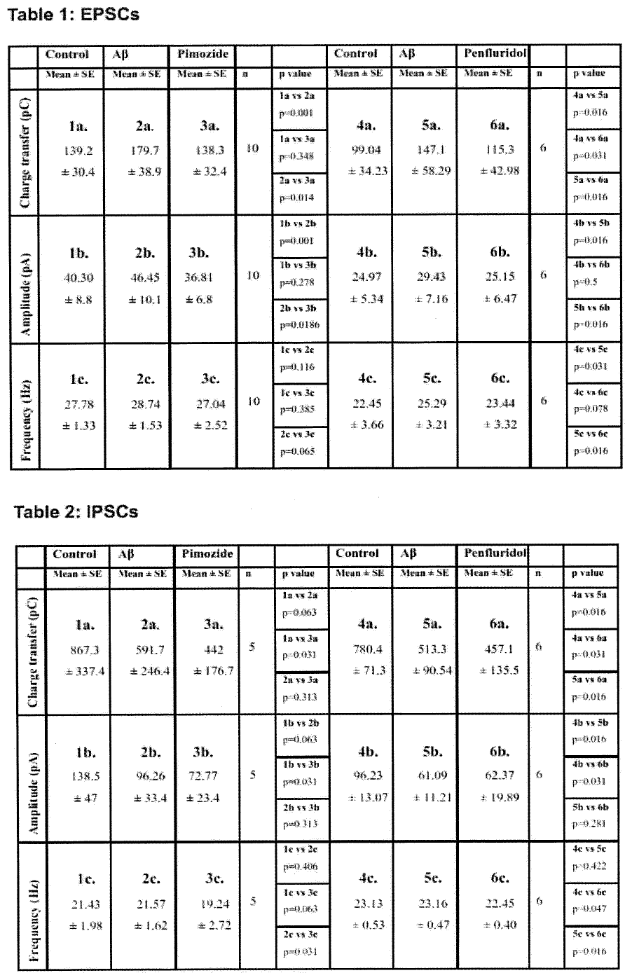

FIG. 12: Inhibition of L-type Ca²⁺ channels fails to rescue Aβ toxicity in mice hippocampal slices. A. Summary bar graph of gamma oscillation power under control condition, Aβ-only and after verapamil treatment showing that inhibition of L-type calcium cannels does not rescue Aβ-induced degradation of cognition-relevant gamma oscillations in mouse hippocampus. Data is presented as mean±SEM. * indicates p<0.05.

Table 1: Data values for EPSC recordings.

Table 2: Data values for IPSC recordings.

### EXAMPLE 1

### T-Type Calcium Channel Inhibition—A Novel Therapeutic Target for Amyloid Diseases in the Brain

Abstract

The amyloid-β (Aβ) cascade hypothesis of Alzheimer's disease (AD) focuses on Aβ peptide aggregation as one of the main culprits for the neuronal dysfunction, synaptic loss and cognitive decline seen in patients during AD development and progression. In the healthy brain excess of peptides are cleared by peptide degradation mechanisms involving the ubiquitin-proteasomal system (UPS). In this study we investigate whether the negative effects of Aβ peptide on cellular, network and cognitive performance can be either prevented or rescued by targeted activation of the proteasome, and if this could be a suitable treatment against amyloid-induced cognitive decline.

The proteasome activator Pimozide was tested on an in vivo Drosophila strain expressing human Aβ and was effective in restoring climbing behaviour and in reducing the number and size of Aβ aggregates in the brain. In electrophysiological in vitro assays in wild-type mice, Pimozide administration prevented and rescued hippocampal network and cellular function impaired by acute application of Aβ. Our experiments showed that Pimozide's beneficial effects are based on the modulation of cellular calcium concentration through the inhibition of voltage-dependent T-type calcium channels. The use of multiple T-type calcium channel inhibitors (Penfluridol, NNC55-0396, ML-218) confirmed the preventative and restorative effects in mice. Using Pimozide and Penfluridol on a novel iPS cell line from an AD patient, we described the developmental differences of this cell line compared to a healthy control line and observed a complete rescue of the passive membrane properties and a restoration of firing ability in the AD cells after treatment.

Our findings demonstrate that T-type calcium channel inhibition causes increased clearance of Aβ aggregates and rescues cellular and network functions important for cognition. The inhibition of these channels might therefore be an effective therapeutic approach for AD and potentially other amyloidogenic brain diseases.

Introduction

Alzheimer's disease (AD) is a progressive neurodegenerative disorder and the most common form of dementia in the elderly. Affecting more than 40 million people worldwide, it is a global health priority since it places an increasing social and economic burden on society (1, 2). Today no disease-modifying treatment for AD exists, and symptomatic therapies are being used with limited effects. Treatment strategies have so far focused on targeting processes of either Aβ production (3) or clearance (4), but the results of clinical trials have been discouraging.

The amyloid cascade-hypothesis dominates AD research: it proposes that toxic amyloid-β peptide (Aβ) is one of the main culprits for the physiological changes seen during progression of AD. These include the desynchronization of action potentials, the consequent development of aberrant brain rhythms relevant for cognition (gamma oscillations, 30-80 Hz) (5, 6, 7, 8), and the final emergence of cognitive deficits in patients (9, 10). The exact cellular mechanisms at the root of these aberrant network oscillations and the neuronal dysfunction in AD remain elusive. Such knowledge is crucial to identify suitable targets for therapeutic attempts at prevention of or rescue from the detrimental affects of amyloidogenic peptide misfolding and aggregation.

Under physiological conditions the brain either prevents excessive protein/peptide formation or removes excessive protein/peptide through the ubiquitin-proteasomal system (UPS) or the autophagy-lysosomal degradation system (APLS) (11). We have selected here a class of Federal Drug Administration (FDA)-approved drugs that can increase proteolysis (43). While the mechanisms of action of these drugs are not fully understood one fundamental cellular process of increasing cellular degradation in neurons is by affecting the calcium homeostasis within the cell (50) (FIG. 1). One specific class of channels that is highly enriched in the brain (12) and can modulate the basal cellar calcium concentration are the voltage-dependent T-type calcium channels (FIG. 2). Together these two features made this class of channels a desirable target for therapeutic strategies against neuropathic pain, ischemia and cancer (13).

Aβ has been shown to change calcium homeostasis, leading to heightened neuronal excitability. In particular it has been confirmed that Aβ peptides increase the cellular influx of calcium, causing excitation (14, 15) and resulting in pre- and postsynaptic modifications (16). To evaluate whether the activation of the proteasome would cause a reduction of Aβ burden we identified a small molecule, FDA-approved drug, Pimozide (17, 43). Pimozide is a drug used to treat psychiatric disorders (antipsychotic) via its action as a blocker of D2 dopaminergic receptors (18). In addition to its action on D2 receptor it inhibits T-type calcium channels in the same concentration range (19).

In this study three models of Alzheimer's disease were used to investigate whether the pharmacological inhibition of T-type calcium channels could reduce Aβ aggregate load and rescue neuronal function and network activity relevant for cognitive processes in the brain. Firstly, we evaluated gain-of-function in vivo by treating a Drosophila melanogaster model expressing pan-neuronally human Aβ₁₋₄₂ peptide (20) with Pimozide. Secondly, we tested Pimozide and three other T-type calcium channel inhibitors (Penfluridol, NNC55-0396, ML-218) on mouse hippocampal slices after acute exposure to Aβ, evaluating rescue of cognition-relevant gamma oscillations as well as cellular and synaptic properties. Thirdly, we electrophysiologically characterized a novel human induced pluripotent stem cell (iPSC) line derived from an AD patient and evaluated the rescue of aberrant parameters after treatment with Pimozide and Penfluridol. Together, our data describe a novel mechanism that when targeted leads to gain-of-function in three relevant models of Alzheimer's disease, suggesting a new therapeutic approach in the fight against cognitive decline in AD and, potentially, other amyloidogenic brain disorders.

Results

Pimozide Restores Behavioral Impairment and Reduces Aβ Aggregation in Flies Expressing Aβ

After initial screening of 14 proteasome-activator compounds based on ability to cross the blood-brain barrier, we selected four of them to be tested on a Drosophila model. The model used was a fly overexpressing pan-neuronally human Aβ₁₋₄₂ peptide carrying the Arctic mutation (Glu22Gly substitution, Arc2E), which is more aggregation-prone and toxic in vitro and accelerates the formation of amyloid deposits in the brain (20, 21). This mutation generates intracellular Aβ accumulation followed by aggregates resembling diffuse plaques. The accumulation is associated with progressive motor deficits and premature death of the flies (20).

We assessed differences in climbing ability performance between w:elavGAL4^{c155}UAS-GFP and w:UAS-Arc2E control flies, w:elavGAL4^{c155}UAS-GFP/w:UAS-Arc2E experimental flies (ElavArcE) and w:elavGAL4^{c155}UAS-GFP/:;UAS-Arc2E flies treated with Pimozide (ElavArc2E+Pmzd). Climbing test was performed on all flies every 5 days during a 35 days experimental period. Our data indicate that around 50% of the w:elavGAL4^{c155} UAS-GFP/w:UAS-Arc2E flies showed climbing deficits starting at 15 days after hatching and their ability to climb degenerated until day 35, when none of the flies was able to climb anymore (FIGS. 3A,B).

In contrast, w:elavGAL4^{c155}UAS-GFP/w:UAS-Arc2E flies treated with Pimozide showed a significantly better performance in the climbing test at days 5, 15 and 25 (FIGS. 3A,B). Thus, at 5 days, 97.4±0.3% of ElavArc2E+Pmzd individuals versus 87.5±0.2% of ElavArcE flies reached the top of the climbing column (p<0.0001); at 15 days, our data show 83.2±0.6% of ElavArc2E+Pmzd individuals against 48.3±0.4% of ElavArc2E flies reaching the top (p<0.0001) and finally, at 25 days, 38.3±0.5% of ElavArc2E+Pmzd flies reached the top in contrast to 18±0.4% in ElavArc2E flies (p<0.0001). Our data indicates a functional improvement in the climbing assay of Pimozide-treated flies expressing Aβ when compared to untreated flies.

In order to identify possible morphoanatomical differences between treated and untreated flies, we dissected 35 day-old brains from ElavArc2E and ElavArc2E+Pmzd flies to analyze the number and size of amyloid plaque deposits. We identified fly brain areas based on all neurons constitutively express the GFP reporter under the pan-neuronal elav Gal4 driver and detected the expression of the human form of Aβ in fly brains using the 6E10 antibody.

Our data show a clear reduction in the number as well as the size of Aβ aggregates in the treated group of animals in the mushroom body area of the Drosophila brain (FIG. 3C). Pimozide significantly reduced the number of aggregates per μm²: ElavArc2E+Pmzd: 0.0049±0.0009 compared to ElavArc2E: 0.017±0.003 (FIG. 1E, p=0.0006). In addition, the size of the aggregates was significantly reduced in animals treated with the drug: ElavArc2E+Pmzd: 0.23±0.02 μm² compared to ElavArc2E: 0.52±0.04 μm² (FIG. 1D, p=0.0006). In conclusion, our results demonstrate that Pimozide treatment reduces Aβ deposits and ameliorates behavioral dysfunction in a Drosophila model expressing human Aβ.

T-Type Ca²⁺ Channel Inhibition as a Mechanism for the Prevention of Aβ-Induced Impairment of Functional Network Dynamics in Mouse Brain Slices

Previously we have characterized the effects of acute Aβ on the hippocampal circuitry of WT mice. We reported that a physiological concentration of Aβ (50 nM) acutely administered degrades gamma oscillation in the hippocampal network (8). We investigated whether the use of the proteasome activator Pimozide could prevent this Aβ-induced disruption of gamma oscillations. Hippocampal slices were incubated for 1 h with Pimozide at 1 or 10 μM. During the last 15 min 50 nM Aβ was added to the incubation solution. After the incubation gamma oscillations were induced in an interface chamber by bath perfusion of Kainate (100 nM, KA, 20 min). Control experiments were also performed by incubating slice just with ACSF for 1 h (with Aβ present for the last 15 min) or incubating just with ACSF for 1 h. In the absence of Aβ and any other compound gamma oscillation power was 10.29±1.95×10⁻⁹V², n=16 (FIGS. 4A,B,C,F, grey). The incubation with only Aβ confirmed the results reported previously by our laboratory (power: 1.24±0.196×10⁻⁹V², n=13, p<0.0001 vs control, FIGS. 4A,B,C,F, red) (8). We found that after incubation with Pimozide the disruption of gamma oscillation induced by Aβ was drastically reduced and in a concentration-dependent fashion (Pmzd 1 μM: 4.22±0.59×10⁻⁹V², n=14, p=0.0003 vs Aβ; Pmzd 10 μM: 9.95±1.82×10⁻⁹V², n=10, p<0.0001 vs Aβ, FIGS. 4A,B,C,F, light and dark green, respectively).

Since Pimozide is known to act on multiple receptor types (dopaminergic and serotonergic in primis), we first performed control experiments to establish through which mechanism Pimozide was able to prevent Aβ-induced degradation. Incubation with the selective 5-HT7 receptor antagonist SB-269970 1 μM (22) did not prevent the gamma oscillation degradation induced by Aβ (1.47±0.24×10⁻⁹V², n=11, p=0.45 vs Aβ, FIG. 4B, light blue). Similar results were obtained with the selective D2/D3 receptor antagonist Sulpiride 5 μM (23) (2.23±0.52×10⁻⁹V², n=12, p=0.33 vs Aβ, FIG. 4B, purple) and with L745,870 (500 nM), a potent D4 receptor antagonist (1.61±0.52×10⁻⁹V², n=15, p=0.58 vs Aβ, FIG. 4B, yellow) (24).

Pimozide is also described as having an inhibiting action on T-type Ca²⁺ channels. When we incubated hippocampal slices with Penfluridol, a T-type Ca²⁺ channel inhibitor (25), we observed similar results to those obtained with Pimozide (6.41±1.68×10⁻⁹V², n=13, p=0.01 vs Aβ, FIG. 4E, blue). The successful use of the much more selective T-type Ca² channel inhibitor Penfluridol proves that the preventive effect of Pimozide on Aβ-induced degradation of gamma oscillations is due to its inhibition of T-type Ca²⁺ channels.

T-Type Ca²⁺ Channel Inhibition Prevents Aβ-Induced Reduction of Rhythmicity of Gamma Oscillations

To further characterize the preventive effect of Pimozide and Penfluridol on gamma oscillation disruption caused by Aβ we studied the coefficient of rhythmicity (Cr) as a measure o gamma oscillations quality (26). It has been reported previously that Aβ reduces the Cr compared to control conditions. This significant reduction in the rhythmicity suggests an alteration in the synchronization of hippocampal cellular activity that has been previously reported by our laboratory (8). In this study we found that the Cr reduction caused by the acute application of Aβ (from 0.54±0.01 in control condition to 0.45±0.02 after Aβ, p=0.001, FIGS. 4D,E, grey and red, respectively) was not completely prevented when slices are incubated with 1 μM Pimozide (0.47±0.016, p=0.65 vs Aβ, FIGS. 4D,E, light green), but it was when slices are incubated with 10 μM Pimozide (0.5±0.02, p=0.07 vs Aβ, FIGS. 4D,E, dark green) or 5 μM Penfluridol (0.51±0.02, p=0.07 vs Aβ, FIGS. 4D,E, blue). These data suggest that the mechanism through which Pimozide and Penfluridol act affects the synchrony of hippocampal cellular activity.

T-Type Ca²⁺ Channel Inhibition Rescues the the Aβ-Induced Degradation of Gamma Oscillations

With view to a potential future clinical application it is important to establish whether a given drug is able to just prevent damage from occurring (prevention) or can restore damage that has already occurred (rescue). From a treatment perspective the latter scenario is of course preferable. We therefore proceeded to investigate the neurorestorative capabilities of Pimozide and Penfluridol.

Hippocampal slices were placed in a submerged recording chamber where gamma oscillations were induced by bath perfusion of 100 nM KA and allowed to stabilize for 20-30 min. Continuous recordings of LFP gamma oscillations were performed in 4 different conditions (FIG. 5). All groups were initially activated with Kainate. In a control group of slices oscillations were recorded for 40 min after stabilization without any other compound addition (control, n=5, grey). All other groups were perfused with 1 μM Aβ after stabilization of the oscillations for a minimum of 20 min. For these experiments the concentration of Aβ applied was increased to 1 μM in order to overcome the method-dependent lower signal amplitude due to the submerged recording conditions (8). After 20 min one group was left for additional 20 min in the same perfusion solution containing only Aβ (Aβ, n=5, red); for the other 2 groups the perfusion solution was enriched with either 10 μM Pimozide (Aβ+Pmzd, n=5, green) or 5 μM Penfluridol (Aβ+Penfl, n=5, blue) for additional 20 min to study the potential rescue effects.

Compared to control gamma oscillation power (4.73±0.75×10⁻⁹V², n=5, FIG. 5A-D, grey), hippocampal slices perfused with Aβ had a constant and fast decay of gamma oscillations, resulting after 40 min in a significant decrease of gamma oscillation power (0.23±0.05 of control, n=5, p=0.004, FIG. 5A-D, red). Gamma power measurement after treatment application (either Pimozide or Penfluridol) showed a significant rescue of the impaired gamma power (Pmzd: 1.96±0.28 of Aβ, n=5, p=0.0476; Penfl: 2.29±0.38 of Aβ, n=5, p=0.0159, FIG. 5A-D, green and blue, respectively). Our results show that both Pimozide and Penfluridol are able to not just prevent but also rescue gamma oscillations previously degraded by Aβ.

T-type Ca²⁺ Channel Inhibition Rescues the Aβ-Induced Shift in Excitation-Inhibition Balance in the Hippocampal Network

In order to understand which cellular mechanism are responsible for the observed prevention and rescue of gamma oscillation power by Pimozide and Penfluridol, we proceeded to investigate potential changes in the excitation-inhibition balance in pyramidal cells. Previously, we showed that Aβ acts to shift the network excitation-inhibition balance by increasing the excitatory postsynaptic currents (EPSCs) and reducing the inhibitory postsynaptic currents (IPSCs) (8). In this study, we recorded spontaneous synaptic activity in hippocampal CA3 pyramidal cells activated with 100 nM KA (20 min) in a submerged recording chamber. Two sets of identical experiments were performed, one using Pimozide (10 μM), one using Penfluridol (5 μM). Application of Aβ (1 μM) resulted in increased glutamate receptor-mediated EPSCs (FIG. 6A): slight or no increase was seen in frequency, while amplitude and charge transfer were significantly increased after Aβ application (Table 1 and FIGS. 6B,C for charge transfer). We found that application of either Pimozide or Penfluridol completely rescued the Aβ induced increase in EPSCs frequency (if present), amplitude and charge transfer (Table 1 and FIGS. 6B,C for charge transfer).

In contrast to its action on EPSCs, application of Aβ resulted, as previously shown, in decreased GABA receptor mediated IPSCs (FIG. 6D). The frequency was not affected by Aβ, while amplitude and charge transfer were both significantly decreased (Table 2 and FIGS. 6E,F for charge transfer). In this case, neither Pimozide nor Penfluridol were able to rescue the effect of Aβ in any of the studied parameters: (Table 2 and FIGS. 6E,F for charge transfer). The selective effect of both Pimozide and Penfluridol on EPSCs but not on IPSCs might explain why the rescue effect on gamma oscillation power is not quite 100%.

T-type Ca²⁺ Channel Inhibition Rescues the Aβ-Induced Desynchronization of Pyramidal Cell Action Potentials

Having studied the synaptic effects of two T-type Ca²⁺ channel inhibitors, we then proceeded to examine their ability to modulate the synchronization of hippocampal neuronal activity. We performed concomitant recordings of field potentials and single unit recordings to analyze the synchronization of action potentials with the phase of the gamma oscillations (27). In parallel with the already described reduction in the power of gamma oscillations after Aβ treatment, Aβ also induced an increase in the AP firing frequency of PC (Pmzd: control: 2.37±0.5 Hz, Aβ: 3.06±0.59 Hz, n=7, p=0.039; Penfl: control: 1.61±0.34 Hz, Aβ: 1.86±0.39 Hz, n=8, p=0.0039, FIGS. 7C,F). To determine the spike phase of PC during a gamma oscillation, spike-phase coupling was calculated. As shown in FIGS. 7A and D, the application of Aβ changed the preferred phase-angle (Pmzd: control: 4.307±0.126 radians, Aβ: 3.813±0.007 radians, n=7, p=0.0078, FIG. 7B; Penfl: control: 4192±0.174 radians, Aβ: 3.685±0.164 radians, n=8, p=0.0039, FIG. 7E). This is observed as a slight shift to the right in the AP-phase distribution (7A,D). The desynchronization of PC activity is also evidenced by the reduction of the summation vector length (Pmzd: control: 0.3764±0.048, Aβ: 0.226±0.05, n=7, p=0.0078, FIG. 7B; Penfl: control: 0.3976±0.054, Aβ: 0.2661±0.05566, n=8, p=0.0078, FIG. 7E), which is represented by the change in the length of the arrow in FIGS. 7A and D.

The application of either T-type Ca²⁺ channel inhibitor after the application of Aβ rescued the AP count (Pmzd: 2.23±0.51 Hz, n=7, p=0.3438; Penfl: 1.79±0.43 Hz, n=8, p=0.098, FIGS. 7C,F) as well as the phase angle (Pmzd: 4.180±0.15 radians, n=7, p=0.23, FIG. 7B; Penfl: 4.09±0.2 radians, n=8, p=0.055, FIG. 7E) and the summation vector length (Pmzd: 0.335±0.072, n=7, p=0.234, FIG. 7C; Penfl: 0.355±0.05, n=8, p=0.09, FIG. 7E). The change in phase angle can be observed as a slight shift back to the left in the AP-phase distribution and the increase of the vector length as an increase in the length of the arrow (7A,D).

This shows that inhibition of the T-type Ca²⁺ channels results in the rescue of the Aβ-induced increase in AP firing and in the rescue of the substantial desynchronization of action potentials in PC caused by Aβ.

Chemically-Different T-type Ca²⁺ Channel Inhibitors Also Have Rescue Effects on Gamma Oscillations Degraded by Aβ

Because Pimozide and Penfluridol share a similar chemical base structure we tested two additional T-type Ca²⁺ channel inhibitors, each with its own distinct chemical structure unrelated to the others: NNC55-0396 (28) and ML-218 (29). Both additional T-type Ca²⁺ channel inhibitors were able to rescue gamma oscillation power after Aβ-induced degradation (NNC55-0396 10 μM: 1.85±0.15 of Aβ, n=5, p=0.0152; ML-218 5 μM: 2.16±0.53 of Aβ, n=5, p=0.0476, FIG. 5C, pink and light blue, respectively). These results further strengthened our finding that T-type Ca²⁺ channel inhibition is the mechanism responsible for the treatment success seen in the Drosophila and mouse models.

Electrophysiological Characterization of Induced Pluripotent Stem (iPS) Cell-Derived Neurons

To be able to evaluate the effects of T-type Ca²⁺ channel inhibitors on human neurons we first set up IPS cell-derived neuronal cultures in the laboratory. Since there have been few publications and no characterization of iPS cell neuronal cultures derived from AD patients we performed a full functional characterization of healthy and patient derived iPS cell cultures. Neurons were generated from iPS cells derived from a healthy patient (AF22) and from a patient with familial Alzheimer's disease (ADPII) as described in Materials and Methods. We studied resting membrane potential (RMP), membrane input resistance (Rᵢₙ) and membrane time constant (τ) at four different time points (T1: day 36-45, T2: day 46-55, T3: day 56-65, T4: day 66-75). With maturation, the expression of some ion channels (for instance K⁺ channels (Kv) and Na⁺ channels (Nav)) induces changes in the passive membrane properties of neurons (30). As shown in FIG. 8Aa the resting membrane potential of AF22 cells became progressively negative over time (T1: −26.17±1.431 mV (N=44); T2: −38.35±1.514 mV (N=46); T3: −41.74±1.196 mV (N=50); T4: −43.47±1.132 mV (N=49)). Over time AF22 also changed their Rᵢₙ (FIG. 8Ab), which progressively decreased, consistently with the increased expression of channels embedded in the membrane (T1: 153.5±17.82 MΩ; T2: 114.6±16.81 MΩ; T3: 113.5±14.23 MΩ; T4: 81.76±8.852 MΩ). No changes were seen over time in the membrane time constant (T1: 525.5±47.39 μs; T2: 524.8±36.89 μs; T3: 562.2±26.81 μs; T4: 525.1±37.05 μs, FIG. 8Ac).

Results for the ADPII cell line were strikingly different (FIG. 8B): An initial decrease in the RMP was seen but after 50 days of differentiation all the cells started to degenerate and went back to their original state of immature neurons (T1: −32.04±1.686 mV (N=49); T2: −36.93±1.338 mV (N=42); T3: −33.48±1.763 mV (N=59); T4: −21.28±0.9124 mV (N=50), FIG. 8Ba). No significant changes were observed in the Rᵢₙ (FIG. 8Bb) or in τ (FIG. 8Bc) over time, except for T4, where the cells were clearly degenerating (Rᵢₙ—T1: 95.17±11.67 MΩ; T2: 94.71±11.74 MΩ; T3: 82.73±9.345 MΩ; T4: 120±14.92 MΩ; τ—T1: 378.1±30.80 μs; T2: 358.4±32.77 μs; T3: 384.1±30.29 μs; T4: 494.5±39.40 μs).

During the maturation process the neurons also become capable of generating action potentials after an induced depolarization and, later in development, spontaneously. We observed a small percentage of AF22 cells (6.8%) capable of generating spontaneous APs at T1. This number increased with time, reaching 44% of the cells by T3. As for ADPII cell, at T1 8.16% of the total patched cells was spontaneously firing. Slowly they percentage increased (T2: 26.19%), but at 60 days of differentiation only 16.95%, number that slowly decreased to 14% by T4 (data not shown). This, together with the changes in passive membrane properties, shows that ADPII cells degenerate after 50-60 days of differentiation, and that they represent a useful tool to test potential pharmacological treatments.

T-Type Ca²⁺ Channel Inhibitors Rescue the Changes in Passive Membrane Properties and AP Firing Observed in the ADPII Cell Line

Having characterized electrophysiologically the iPS cells derived from a patient with familial Alzheimer's disease, we proceeded to use them as an assay to test our compounds of interest. The treatment was started during T3: both AF22 and ADPII coated coverslips were treated either with vehicle control DMSO or with 1 μM Pimozide or 1 μM Penfluridol for 48 hours. Cells were then recorded as done for the characterization of the two lines. Rᵢₙ (FIGS. 9Ab, 9Bb) and τ (FIGS. 9Ac, 9Bc) did not show any significant changes in either the AF22 or the ADPII line with any drug treatment (Rᵢₙ—AF22, DMSO: 98,37±17.60 MΩ (N=54); AF22, Pmzd: 93.75±10.65 MΩ (N=54); AF22, Penfl: 92.61±10.90 MΩ (N=50); ADPII, DMSO: 101.6±11.08 MΩ (N=54); ADPII, Pmzd: 93.67±6.954 MΩ (N=54); ADPII, Penfl: 103.6±12.69 MΩ (N=50); τ—AF22, DMSO: 571.6±41.42 μs; AF22, Pmzd: 578.7±30.50 μs; AF22, Penfl: 599.2±51.87 μs; ADPII, DMSO: 520.4±36.05 μs; ADPII, Pmzd: 517.4±27.55 μs; ADPII, Penfl: 547.9±39.01 μs).

In contrast, RMP (FIG. 9Aa, FIG. 9Ab) in the ADPII line was drastically rescued back to healthy control values when cells where treated with either of the two T-type Ca²⁺ channel inhibitors ((ADPII, DMSO: −30.13±1.229 my; ADPII, Pmzd: −42.26±1.341 mV, p<0.0001); (ADPII, DMSO: −30.13±1.229 mV; ADPII, Penfl: −42.29±1.124, p<0.0001)). No negative effects where seen on AF22 cells when treated with just DMSO (AF22, DMSO: −45.07±1.261 mV), Pimozide (AF22, Pmzd: −44.91±1.319 mV) or Penfluridol (AF22, Penfl: −42.99±1.134 mV) (FIGS. 9Aa,Ba).

In addition we studied the ability to generate spontaneous AP in the treated cells: while no significant changes were seen in the AF22 line in presence of any of the drugs (AF22, no treatment: 44%; AF22, DMSO: 43.75%; AF22, Pmzd: 44.9%; AF22, Penfl: 50%), significant improvements in the ADPII line were observed (ADPII, no treatment: 16.95%; ADPII, DMSO: 28%; ADPII, Pmzd: 52%, Penfl: 43.18%, data not shown).

After treating the patient-derived AD neurons with two T-type Ca²⁺ channel inhibitors, we could rescue the electrophysiological parameters back to control levels, while the healthy control cultures showed no side effects from the drugs themselves.

Discussion

The results described in this study demonstrate that the inhibition of T-type calcium channels might be an effective therapeutic approach for Alzheimer's disease and, potentially, other amyloidogenic brain disorders.

We believe that the reduction of the Aβ aggregates seen in this study might be due to the activation of the proteasome as a consequence of the T-type calcium channels inhibition. The ubiquitin-proteasome system is the main cellular machinery responsible for the degradation of misfolded, defective and aggregation-prone proteins, and its function becomes gradually impaired in many neurodegenerative disorders as well as in normal aging (40, 41, 42). As shown in this study, Pimozide's action on different receptors (dopaminergic and serotoninergic) has no beneficial effects on Aβ-induced cellular and network degradation. The T-type calcium channel inhibition seems to be at the origin of the preventive and restorative effects reported.

In this study three different models of Alzheimer's disease were used: the Drosophila melanogaster model allows the expression of a human form of Aβ₁₋₄₂ (Glu22Gly substitution, Arctic mutation, Arc2E flies) that accelerates aggregation and greatly increases neurotoxicity (44). The very clear and well-described phenotype, the rapid progression of the disease and the low cost of fly cultures favour a rapid screening of potential compounds both at cellular and behavioural level. Even though the phenotype of these AD flies is very severe and they manifest a locomotor dysfunction at very early ages, the Pimozide treatment yields to a great improvement in their climbing performance. More interestingly, we demonstrated that a functional improvement was possible due to a reduction in the Aβ aggregates burden in vivo.

Since measurements related to cognitive decline cannot be obtained from Drosophila, we decided to evaluate the drugs in a more befitting model. Rhythmic electrical activity in the gamma frequency range can serve as a functional biomarker for diagnosis in the clinic, and being easily measurable in mice as well (in vitro and in vivo) serves as a useful tool to test the efficacy of drugs in preventing or rescuing the effects of Aβ. In this study we replicated previously published results (8) on the Aβ effects on gamma oscillations and excitatory/inhibitory balance, and we proved that all these aspects could be rescued inhibiting the T-type calcium channels.

To bring all these results a step closer to what the ultimate goal of AD research is, we used iPS cells derived from AD patients, differentiating them into neurons. Studying the neurophysiological properties of live single neurons in the human brain is challenging and until recently has been restricted to animal models (45). The use of iPSC-derived neurons is a bridge between these animal models and the human brain itself. This model in fact recapitulates in vitro the complexity of functional human brain circuits. However, being iPS cells a relatively new tool in research, a lot more basic characterization needs to be performed.

We believe our work has led to the discovery of a novel mechanism that restores function impaired by an amyloidogenic peptide in three different AD models. Our approach differs from many other treatment strategies since we use small-molecule compounds that have been already FDA approved and have a long usage history in the fight against various brain-associate diseases unrelated to neurodegenerative diseases. Moreover, these compounds easily cross the blood-brain barrier. Our results offer a straightforward path towards translation to clinical trials and, if successful there, clinical practice. Novel therapies against neurodegenerative amyloid diseases are sourly needed because of the cost these diseases place on patients, their families and society.

Materials and Methods

Drosophila

Genetics and Stocks

Line w elav-Gal4^{c155}UAS-GFP was kindly provided by Alberto Ferrús (Cajal Institute, Madrid) (46) whereas the w; UAS-Arc2E stock carrying a human Aβ42 Arctic mutation (Glu22Gly substitution) was obtained from Damien C. Crowther (Neuroscience, Innovative Medicines and Early Development, AstraZeneca, Granta Park, Cambridge, CB21 6GH, UK) (20, 21). Control flies were of the genotype w; elav-Gal4^{c155} UAS-GFP and w; UAS-Arc2E. Expression of the human Artic mutation was achieved through the UAS/GAL4 system (47) to obtain w elavGAL4^{c155}UAS-GFP/w; UAS-Arc2E experimental flies. The elav-GAL4 allows a paraneural expression of the Arctic mutation.

All control and experimental flies were maintained on standard flour/agar fly food (H₂O, pure D(+)Glucose (Panreac), Instant Yeast (Anchor), Agar (Pronadisa), commercial flour (Gallo), Propionic Acid (Merck)) or with Pimozide 5 μM added to the standard food. All flies were maintained at 23-25° C. with a 12 h:12 h light:dark cycle. Food vials were changed every 2-3 days. Drug treatment (Pimozide, 5 μM (in DMSO, final dilution in standard food)) started at day 1 after hatching and continued throughout the all study length (35 days).

Negative Geotaxis Climbing Assay

Control (w elavGAL4^{c155} UAS-GFP and w; UAS-Arc2E) flies as well as experimental (w elavGAL4^{c155}UAS-GFPlw; UAS-Arc2E and w elavGAL4^{c155}UAS-GFP/w; UAS-Arc2E treated with Pimozide) flies were all kept at 23-25° C. divided in groups of 10-15 in 9 cm plastic vials with new food every 2-3 days. Both wild type and experimental flies were divided in groups with food containing the test compound from day 1 after hatching for all the length of the study (35 days) and groups with standard food containing just the vehicle in which the compound was dissolved. Viable flies were counted daily to assess differences in longevity between wild type and experimental flies and between experimental not treated or treated with the compounds. Flies display a negative geotaxis response when given a mechanical stimulus. When tapped to the bottom of a vial, flies normally orient themselves rapidly and begin to climb to the top. By assaying the fly's ability to climb over a 10 cm line in an 18 cm vial in set time period (10 sec) we were able to compare broad nervous system function of reflex behaviors between flies with different genotypes and/or treated with different drugs. The number of flies at the top or bottom of the vial (i.e. flies able to cross a 2 cm line vs. flies not able to reach the line and/or cross it) was scored after 10 seconds. 10 trials were performed for each condition and each time point. The data shown represent results from a cohort of flies tested every 10 days for 35 days. Data are presented as average±SEM. Results were tested for significance using ANOVA with Bonferroni Post Test to compare all different groups.

Whole-Mount Immunohistochemistry and Confocal Microscopy

Flies at 35 days of age of all genotypes were dissected (proboscis were removed from decapitated heads) in PBS buffer. Whole brains were fixed in 4% PFA (in PBS, pH 7.4) for 45 minutes: PFA solution was changed every 10 minutes. Following fixation, brains were washed for 30 minutes in 300 μM Glycine to avoid background staining due to the use of PFA. Brains were then washed 3 times with ice cold PBS containing 0.2% Triton X-100 for a total of 30 minutes to permeabilize the membranes. Subsequently they were blocked in 3% normal goat serum (NGS) for 2 hours at room temperature with gentle shake. Fly brains were then incubated overnight at 4° C. with gentle shake in mouse anti Aβ (6E10, BioLegend) diluted 1:800 in blocking buffer. After four further washes of 15 minutes at room temperature in PBST with gentle shake, brains were incubated in goat anti-mouse IgG Alexa 647 (Invitrogen). After 4 washes (15 minutes each) in PBST and 4 washes in PBS at room temperature with gentle shake, the brains were mounted in Vectashield (Vectorlabs) anti-fade mounting medium.

Confocal serial scanning images were acquired at 1-μm intervals with a Leica Confocal Microscope TCS SP5 II (Mannheim, Germany). Images were processed with using ImageJ (NIH): number of aggregates and respective sizes were measured and statistic was performed with GraphPad Prism (GraphPad Software, USA).

Mice

Experiments were performed in accordance with the ethical permit granted by Norra Stockholms Djurförsöksetiska Nämnd to André Fisahn (N45/13). C57BU6 male mice (postnatal days 14-30, supplied from Charles River Laboratories, Germany) were used in this study. Animals were deeply anesthetized using isofluorane before being sacrificed by decapitation.

Drugs and Chemicals

All chemical compounds used in intracellular and extracellular solutions were obtained from Sigma-Aldrich Sweden AB (Stockholm, Sweden). Receptor antagonists and channel blockers were obtained from Tocris Bioscience (Bristol, UK) or Sigma-Aldrich Sweden AB (Stockholm, Sweden). Pimozide (Pmzd), Penfluridol (Penfl) and ML-218 were dissolved in DMSO 100%. SB-269970, L745,870 Trihydrochloride (L-745), NNC55-0396 and Kainate (KA) were dissolved in Milli-Q water.

Recombinant Aβ₁₋₄₂ was used in this study. Expression and purification of Aβ was previously reported (8). Briefly, Met-Aβ₁₋₄₂ was expressed in Escherichia coil BL21 from synthetic genes and purified in batch format using ion exchange and passed through a 30000 Da Vivas-pin concentrator filter (Sartorius Stedim Biotech GmbH) to remove large aggregates. Purified peptide was concentrated to 50-100 μM, aliquoted in low-bind Eppendorf tubes (Axygene) and stored at −20° C. until use. Before use Aβ was thawed on ice and briefly sonicated 10 min before application.

Hippocampal Slice Preparation

The brain was dissected out and placed in ice-cold artificial cerebrospinal fluid (ACSF) modified for dissection containing (in mM) 80 NaCl, 24 NaHCO₃, 25 glucose, 1.25 NaH₂PO₄, 1 ascorbic acid, 3 Na pyruvate, 2.5 KCl, 4 MgCl₂, 0.5 CaCl2, 75 sucrose and bubbled with carbogen (95% O₂ and 5% CO₂). Horizontal sections (350 μm thick) of the ventral hippocampi of both hemispheres were prepared with a Leica VT1200S vibratome (Leica Microsystems). Immediately after cutting, slices were transferred into a humidified interface holding chamber containing standard ACSF (in mM): 124 NaCl, 30 NaHCO₃, 10 glucose, 1.25 NaH₂PO₄, 3.5 KCl, 1.5 MgCl₂, 1.5 CaCl₂, continuously supplied with humidified carbogen. The chamber was held at 34° C. during slicing and subsequently allowed to cool down to room temperature (˜22° C.) for a minimum of 1 hour.

Electrophysiology

Recordings were carried out in hippocampal area CA3 with borosilicate glass microelectrodes, pulled to a resistance of 3-7 MΩ. Local field potential (LFP) were recorded using microelectrodes filled with ACSF placed in CA3 stratum pyramidale. LFP oscillations were elicited by applying kainic acid (100 nM) to the extracellular bath, Interface chamber LFP recordings were performed with a 4-channel M102 amplifier (University of Cologne, Germany). Submerged chamber LFP recordings and patch-clamp recordings were performed using a Multiclamp 700B (Molecular Devices, CA, USA). In order to maintain stable LFP oscillations all recordings were performed at 34° C. with a perfusion rate of 3-5 ml per minute of aerated ACSF containing 100 nM Kainate. The oscillations were allowed to stabilize for at least 20 minutes before any recordings were performed.

Patch-clamp (whole-cell) recordings were performed from visually identified CA3 PC using The SliceScope (Scientifica, UK). Action Potentials (APs) were recorded from pyramidal cells in area CA3 as single units (submerged chamber) using standard ACSF-containing patch electrodes. For EPSC recordings (Vₕ=−70 mV) a potassium-based intracellular solution was used (in mM): 122.5 K-gluconate, 17.5 KCl, 4 ATPNa, 0.4 GTPNa, 10 HEPES, 0.2 EGTA, 2 MgCl, set to pH 7.2-7.3 with KOH, osmolarity 270-280 mOsm. For IPSC recordings (Vₕ=0 mV) a cesium-based intracellular solution was used (in mM): 140 CsMeSO₄, 10 HEPES, 0.2 EGTA, 4 MGCl, 2 ATPNa, 0.2 GTPNa, 5 QX-314, set to pH 7.2-7.4 with CsOH, osmolarity 270-280 mOsm.

The signals were sampled at 10 kHz, conditioned using a Hum Bug 50 Hz noise eliminator (Quest Scientific, North Vancouver, BC, Canada), software low-pass filtered at 1 kHz, digitized and stored using a Digidata 1440A and pCLAMP 10.4 software (Molecular Devices, CA, USA).

Data Analysis

Power spectra density plots (from 60 s long LFP recordings) were calculated in averaged Fourier-segments of 8192 points using Axograph X (Kagi, Berkeley, Calif., USA). Gamma oscillation power was calculated by integrating the power spectral density between 20 and 80 Hz. Coefficient of rhythmicity (Cr) was calculated in order to assess the rhythmicity of gamma oscillations (26, 27) and was defined as Cr=(α−β)/(α+β) were α corresponds to the value of the height of the second peak and β to the first valley in the normalized autocorrelogram. Cr ranges between 0 and 1: the higher the coefficient the more rhythmic the oscillation is. Only recordings having Cr≥0.01 were considered rhythmic.

EPSCs and IPSCs were detected off-line using MiniAnalysis software (Synaptosoft, Decatur, Ga., USA). Charge transfer, event amplitude and inter-event-interval (IEI) were analyzed using Microsoft Excel for Mac 2011 (Microsoft Office) and GraphPad Prism (GraphPad Software, USA) with the result representing average values taken over 1 min periods.

Spike phase-coupling analysis was performed on concomitant LFP recordings and single unit recordings using MATLAB custom-written routines in order to relate the PC spiking activity to ongoing gamma oscillations (48). To do this LFP recordings were pre-processed using a band pass filter set to 20-60 Hz (highpass: RC-single pole, lowpass: RC-single pole) using Clampfit 10.7. AP were detected using an amplitude threshold and the instantaneous phase of gamma oscillation was calculated using a Hilbert transform in order to determine the phase-angle at which each action potential occurred during ongoing oscillations. Phase-angles and gamma oscillations-phases were represented in polar plots and expressed in radians with the peak of the oscillation cycle corresponding to 0π and the valley corresponding to ±π in the polar plots. In order to search for the synchronization level of AP firing, AP phase-angles frequency-distribution were normalized, a Gaussian function was fitted and the half-width at half-maximum was then calculated as a measure of the synchronization level: the more AP are fired on the same phase-angle the more synchronized the neuronal activity is. The preferred phase-angle was calculated by averaging the AP phase-angles distribution of all the experiments and is represented by an arrow in the polar-plots. To test whether neurons fired in a phase-related manner all concomitant recordings were tested for circular uniformity using Rayleigh's test. Only recording with p values below 0.05 were considered for the analysis.

Statistical Analysis

All statistical analysis was performed using GraphPad Prism. Results are reported as mean±SEM. Time course data were binned and analyzed over 1 min periods. All data was then normalized to the average of the 5 min control recording before treatment application. Gamma power time-course is represented in absolute values and tests for statistical significance were performed on the last five minutes recordings of each condition. EPSCs and IPSCs time-courses are represented normalized for comparison purposes between the different experimental conditions.

Tests for statistical significance were performed on absolute values in all experiments using Wilcoxon matched-pairs test for paired data and Mann-Whitney U test for unpaired data. Significance levels were set as * p<0.05; ** p<0.01; *** p<0.001.

Patient Cell Lines

Patient-Specific Neuroepithelial Stem Cells

Patient-specific neuroepithelial stem (NES) cell lines were generated and validated by The Swedish National IFS Core Facility and the Falk Laboratory at the Karolinska Institute in Stockholm, Sweden. Skin fibroblasts from a healthy patient (AF22) and a patient with familial Alzheimer's disease (ADPII) carrying a mutation in the APP gene (APP-V717I) were reprogrammed to induced pluripotent stem cells (iPSCs), then to NES as previously described (49). Three vials of frozen low passage AF22-NES and ADPII-NES were obtained from different fully validated batches for each line. The donors of skin fibroblasts employed in this study provided written informed consent concerning the sampling, generation, and use of the iPSC derived NES cell lines (AF22 and ADPII). Ethical permission for human cell reprogramming was granted (dnr 2012/208-31/3, addendum 2012/856-32) and all experiments performed were in accordance with the regulations at the Karolinska Institute and in Sweden.

NES Cell Culture

All cells were cultured in 37° C. with 5% CO₂. All cell culture flasks and plates were freshly coated with poly-L-ornithine (20 μg/mL, Sigma-Aldrich) and laminin (20 μg/mL, Sigma-Aldrich) in PBS just prior to use. Low passage AF22-NES and ADPII-NES were cultured in T12.5 (VWR) and T25 cell culture flasks (Corning) for expansion. Cell density and passage number were carefully maintained to avoid spontaneous differentiation. Flasks were split at a 1:2 ratio with either TrypLE Express (Life Technologies) or Trypsin-EDTA (Thermo Fisher Scientific) and soy-bean trypsin inhibitor (Life Technologies), washed in NES Wash Media ((DMEM/F12 with Glutamax (Gibco), cell culture grade bovine serum albumin (1:100, Sigma-Aldrich) sterile filtered through 0.22 μM membrane (Thermo Fisher Scientific)), centrifuged for 3 minutes (1000×g), counted (BioRad TC20 Automated Cell Counter), and plated in a freshly coated flask at density of 50,000 per square centimeter. Stem cells were fed daily by removing two thirds of the stem cell media and carefully replacing it with fresh warm stem cell media containing: DMEM/F12 with Glutamax (Gibco); penicillin/streptomycin (1:100, Gibco); 100× N2 supplement (1:100, Gibco); 50× B27 supplement (1:1000, Gibco); human recombinant bFGF (10 ng/ml, Life Technologies); and human recombinant EGF (10 ng/ml, Peprotech).

Neuronal Differentiation

Low passage NES-AF22 and NES-ADPII stem cells were plated on freshly coated 12 mm glass coverslips (Thermo) in 24 well plates (Corning) at a density of 50,000 cells per square centimeter (1.2×10′5 cells per well) in NES differentiation medium containing: DMEM/F12 with Glutamax (Gibco); penicillin/streptomycin (1:100, Gibco); 100× N2 supplement (1:100, Gibco); and 50× B27 supplement without vitamin A (1:100, Gibco) for the first 14 days of differentiation. The day of plating is considered differentiation Day 0. Differentiating cells were fed every 48 hours during the first 14 days of differentiation and every 72 hours after day 14. At differentiation day 14, the B27 supplement includes vitamin A (1:100, Gibco) and laminin (1:1000, Sigma) is added to the NES differentiation medium. Wild type (AF22) and AD (ADPII) patient cells were differentiated until specified differentiation time points (T1 Day 36-45; T2 Day 46-55; T3 Day 56-65; and T4 Day 66-75) when they were used for electrophysiology, treated with compounds, and/or used for immunohistochemistry.

Pimozide and Penfluridol Treatment

Pimozide and Penfluridol (Sigma) powder were dissolved to 10 mM stock concentrations in pure cell grade DMSO (Sigma), then aliquoted, and stored at −20° C. until use. Fresh stocks (10 mM) were further diluted 1:1 in DNase/RNase free water, heated and vortexed. Warm differentiation media was used to dilute the 5 mM DMSO:water solution to 100 μM, then finally to 1 μM. Differentiating wild type (AF22) and AD (ADPII) coverslips were treated with vehicle control DMSO and 1 μM Pimozide or Penfluridol for 48 hours during the T3 differentiation time point (Day 56-65) prior to electrophysiological or immunohistochemical assessment.

### EXAMPLE 1 REFERENCES

1. C. Qiu, M. Kivipelto, E. von Strauss, Epidemiology of Alzheimer's Disease: Occurrence, Determinants, and Strategies toward Intervention. Dialogues Clin. Neurosci. 11, 111-128 (2009).
2. C. Takizawa, P. L. Thompson, A. van Walsem, C. Faure, W. C. Maier, Epidemiological and economic burden of Alzheimer's disease: a systematic literature review of data across Europe and the United States of America. J. Alzheimers Dis. 43, 1271-1284 (2015).
3. J. Hardy, The amyloid hypothesis for Alzheimer's disease: a critical reappraisal. J. Neurochem. 100, 1129-1134 (2009).
4. A. Kurz, R. Peerneczky, Amyloid clearance as a treatment target against Alzheimer's disease. J. Alzheimers Dis. 2, 61-73 (2011).
5. W. Singer, Synchronization of cortical activity and its putative role in information processing and learning. Annu. Rev. Physiol. 55, 349-374 (1993).
6. J. E. Driver, C. Racca, M. O. Cunningham, S. K. Towers, C. H. Davies, M. A. Whittington, F. E. LeBeau, Impairment of Hippocampal Gamma (γ)-Frequency Oscillations in Vitro in Mice Overexpressing Human Amyloid Precursor Protein (APP). Eur. J. Neurosci. 26, 1280-1288 (2007).
7. H. Balleza-Tapia, A. Huanosta-Gutiérrez, A. Márquez-Ramos, N. Arias, F. Peña, Amyloid β Oligomers Decrease Hippocampal Spontaneous Network Activity in an Age-Dependent Manner. Curr. Alzheimer Res. 7, 453-462 (2010).
8. F. R. Kurudenkandy, M. Zilberter, H. Biverstål, J. Presto, D. Honcharenko, R. Strömberg, J. Johansson, B. Winblad, A. Fisahn, Amyloid-β-induced action potential desynchronization and degradation of hippocampal gamma oscillation is prevented by interference with peptide conformation change and aggregation. J. Neurosci. 34, 11416-11425 (2014).
9. J. Naslund, V. Haroutunian, R. Mohs, K. L. Davis, P. Davies, P. Greengard, J. D. Buxbaum, Correlation between Elevated Levels of Amyloid Beta-Peptide in the Brain and Cognitive Decline. Jama 283, 1571-1577 (2000).
10. D. M. Walsh, D. J. Selkoe, A beta oligomers—a decade of discovery. J. Neurochem. 101, 1172-1184 (2007).
11. Y. Ihara, M. Morishima-Kawashima, R. Nixon, The ubiquitin-proteasome system and the autophagic-lysosomal system in Alzheimer disease. Cold Spring Harb. Perspect Med. 2 pii: a006361 (2012).
12. E. Cheng, H. S. Shin, T-type Ca2+ channels in normal and abnormal brain functions. Physiol. Rev. 93, 961-992 (2013).
13. B. J. Kopecky, R. Liang, J. Bao, T-type calcium channel blockers as neuroprotective agents. Pflugers Arch. 466, 757-765 (2014)
14. D. Hermann, M. Mezler, M. K. Muller, K. Wicke, G. Gross, A. Draguhn, C. Bruehl, V. Nimmrich, Synthetic Aβ oligomers (Aβ(1-42) globulomer) modulate presynaptic calcium currents: prevention of Aβ-induced synaptic deficits by calcium channel blockers. Eur. J, Pharmacol. 702, 44-55 (2013).
15. M. Ramsden, Z. Henderson, H. A. Pearson, Modulation of Ca2+ channel currents in primary cultures of rat cortical neurons by amyloid beta protein (1-40) is dependent on solubility status. Brain Res. 956, 254-261 (2002).
16. V. Nimmrich, U. Ebert, Is Alzheimer's disease a result of presynaptic failure? Synaptic dysfunctions induced by oligomeric beta-amyloid. Rev. Neurosci, 20, 1-12 (2009).
17. M. Bancila, J. C. Copin, Y. Daali, B. Schatlo, Y. Gasche, P. Bijlenga, Two structurally different T-type Ca²⁺ channel inhibitors, mibefradil and Pimozide, protect CA1 neurons from delayed death after global ischemic in rats. Fundam. Clin. Pharmacol. 25, 469-478 (2010).
18. E. Richelson, T. Souder, Binding of antipsychotic drugs to human brain receptors focus on newer generation compounds. Life Sci. 68, 29-39 (2000).
19. C. M. Santi, F. S. Cayabyab, K. G. Sutton, J. E. McRory, J. Mezeyova, K. S. Hamming, D. Parker, A. Stea, T. P. Snutch, Differential inhibition of T-type calcium channels by neuroleptics. J. Neurosci. 22, 396-403 (2002).
20. D. C. Crowther, K. J. Kinghom, E. Miranda, R. Page, J. A. Curry, F. A. Duthie, D. C. Gubb, D. A. Lomas, Intraneuronal Abeta, non-amyloid aggregates and neurodegeneration in a Drosophila model of Alzheimer's disease. Neuroscience 132, 123-135 (2005).
21. K. Iijima, H. C. Chiang, S. A. Hearn, I. Hakker, A. Gatt, C. Shenton, L. Granger, A. Leung, K. Iijima-Ando, Y. Zhong, Aβ42 mutants with different aggregation profiles induce distinct pathologies in Drosophila. PLoS One 3, e1703 (2008).
22. P. J. Lovell, S. M. Bromidge, S. Dabbs, D. M. Duckworth, I. T. Forbes, A. J. Jennings, F. D. King, D. N. Middlemiss, S. K. Rahman, D. V. Saunders, L. L. Collin, J. J. Hagan, J. Riley, D. R. Thomas, A novel, potent and, selective 5-HT(7) antagonist: ®-3-(2-(2-(4-methylpiperidin-1-yl)ethil)pyrrolidine-1-sulfonyl) phenol (SB-269970). J. Med. Chem. 43, 342-345 (2000).
23. C. F. Caley, S. S. Weber, Sulpiride: an antipsychotic with selective dopaminergic antagonist properties. Ann. Pharmacother. 29, 152-160 (1995).
24. S. Patel, S. Freedman, K. L. Chapman, F. Emms, A. E. Fletcher, R. Marwook, G. Mcallister, J. Myers, N. Curtis, J. J. Kulagowski, P. D. Leeson, M. Ridgill, M. Graham, S. Matheson, D. Rathbone, A. P. Watt, L. J. Bristow, N. M. Rupniak, E. Baskin, J. J. Lynch, C. I. Regal, Biological profile of L-745,870, a selective antagonist with high affinity for the dopamine D4 receptor. J. Pharmacol. Exp. Ther. 283, 636-647 (1997).
25. J. J. Enyeart, B. A. Biagi, B. Milner, Preferential block of T-type calcium channels by neuroleptics in neural crest-derived rat and human C cell lines. Mol. Pharmacol. 42, 364-372 (1992).
26. L. Cangiano, S. Grillner, Fast and slow locomotor burst generation in the hemispinal cord of the lamprey. J. Neurophys. 89, 2931-1942 (2003).
27. R. Andersson, M. Lindskog, A. Fisahn, Histamine H3 receptor activation decreases kainite-induced hippocampal gamma oscillations in vitro by action potential desynchronization in pyramidal neurons. J. Physiol. 588, 1241-1249 (2010).
28. P. H. Bui, A. Quesada, A. Handforth, O. Hankinson, The mibefradil derivative NNC55-0396, a specific T-type calcium channel antagonist, exhibits less CYP3A4 inhibition than mibefradil. Drug. Metab. Dispos. 36, 1291-1299 (2008).
29. Z. Xiang, A. D. Thompson, J. T. Brogan, M. L. Schulte, B. J. Melancon, D. Mi, L. M. Lewis, B. Zou, L. Yang, R. Morrison, T. Santomango, F. Byers, K. Brewer, J. S. Aldrich, H. Yu, E. S. Dawson, M. Li, O. McManus, C. K. Jones, J. S. Daniels, C. R. Hopkins, X. S. Xie, P. J. Conn, C. D. Weaver, C. W. Lindsley, The discovery and characterization of ML218: a novel, centrally active T-type calcium channel inhibitor with robust effects in STN neurons and in a rodent model of Parkinson's disease. ACS Chem. Neurosci. 2, 730-742 (2011).
30. D. Pré, M. W. Nestor, A. A. Sproul, S. Jacob, P. Koppensteiner, V. Chinchalongporn, M. Zimmer, A. Yamamoto, S. A. Noggle, O. Arancio, A time course analysis of the electrophysiological properties of neurons differentiated from human induced pluripotent stem cells (iPSCs). PLoS One 9, e103418(2014).
31. W. V. Goodison, V. Frisardi, P. G. Kehoe, Calcium channel blockers and Alzheimer's disease: potential relevance in treatment strategies of metabolic syndrome. J. Alzheimers Dis. 30, 269-282 (2012)
32. Z. S. Khachaturian, Hypothesis on the regulation of cytosol calcium concentration and the aging brain. Neurobiol. Aging 8, 345-346 (1987).
33. N. Arispe, H. B. Pollard, E. Rojas, Giant multilevel cation channels formed by Alzheimer's disease amyloid beta-protein (Abeta(1-40)) in bilayer membranes. Proc. Natl. Acad. Sci. USA 90, 10573-10577 (1993).
34. A. Demuro, E. Mina, R. Kayed, S. C. Milton, I. Parker, C. G. Glabe, Calcium dysregulation and membrane disruption as a ubiquitous neurotoxic mechanism of soluble amyloid oligomers. J. Biol. Chem. 280, 17294-19300 (2005).
35. H. Kadowaki, H. Nishito, F. Urano, C. Sadamitsu, A. Matsuzawa, K. Takeda, H. Masutani, J. Yodoi, Y. Urano, T. Nagano, H. Ichijo, Amyloid beta induces neuronal cell death through ROS-mediated ASK1 activation.o Cell Death Differ. 12, 19-24 (2005).
36. J. T. Yu, R. C. Chang, L. Tan, Calcium dysregulation in Alzheimer's disease: from mechanisms to therapeutic opportunities. Prog Neurobiol. 89, 240-255. (2009).
37. N. Pierrot, P. Ghisdal, A. S. Caumont, J. N. Octave, Intraneuronal amyloid-beta 1-42 production triggered by sustained increase of cytosolic calcium concentration induces neuronal death. J. Neurochem. 88, 1140-1150 (2004).
38. J. R. Lopez, A. Lyckman, S. Oddo, F. M. Laferla, H. W. Quefurth, A. Shtifman, Increased intraneuronal resting [Ca2+] in adult Alzheimer's disease mice. J. Neurochem. 105, 262-271 (2009).
39. T. S. Anekonda, J. F. Quinn, Calcium channel blocking as a therapeutic strategy for Alzheimer's disease: the case for israpidine. Biochim. Biophys. Acta 1812, 1584-1590 (2011)
40. D. C. Rubinsztein, The roles of intracellular protein-degradation pathways in neurodegeneration, Nature. 443,780-786 (2006)
41. S. Oddo, The ubiquitin-proteasome system in Alzheimer's disease. J. Cell Mol. Med. 12, 363-373 (2008).
42. B. M. Riederer, G. Leuba, A. Vernay, I. M. Riederer, The role of the ubiquitin proteasome system in Alzheimer's disease. Exp. Biol. Med. (Maywood). 236, 268-276 (2011)
43. Y. Leestemaker, A. de Jong, K. F. Witting, R. Penning, K. Schuurman, B. Rodenko, E. A. Zaal, B. van de Kooij, S. Laufer, A. J. R. Heck, J. Borst, W. Scheper, C. R. Berkers, H. Ovaa, Proteasome activation by small molecules. Cell. Chem. Biol. 24, 725-736 (2017).
44. B. M. Whalen, D. J. Selkoe, D. M. Hartley, Small non-fibrillar assemblies of amyloid beta-protein bearing the Arctic mutation induce rapid neuritic degeneration. Neurobiol Dis 20, 254-266 (2005).
45. C. Bardy, M. van den Hurk, B. Kakaradov, J. A. Erwin, B. N. Jaeger, R. V. Hernandez, T. Eames, A. A. Paucar, M. Gorris, C. Marchand, R. Jappelli, J. Barron, A. K. Bryant, M. Kellogg, R. S. Lasken, B. P. F. Rutten, H. W. M. Steinbusch, G. W. Yeo, F. H. Gage, Predicting the functional states of human iPSC-derived neurons with single-cell RNA-seq and electrophysiology. Mol. Psych. 21, 1573-1588 (2016).
46. D. M. Lin, C. S. Goodman, Ectopic and increased expression of Fasciclin II alters motoneuron growth cone guidance. Neuron 13, 507-523 (1994).
47. A. H. Brand, N. Perrimon, Targeted gene expression as a means of altering cell fates and generating dominant phenotypes. Development. 118, 401-415 (1993).
48. R. Andersson, A. Johnston, A. Fisahn, Dopamine D4 receptor activation increases hippocampal gamma oscillations by enhancing synchronization of fast-spiking interneurons. PLoS One 7, e40906 (2012).
49. A. Falk, P. Koch, J. Kesavan, Y. Takashima, J. Ladewig, M. Alexander, O. Wiskow, J. Tailor, M. Trotter, S. Pollard, A. Smith, O. Brüstle, Capture of neuroepithelial-like stem cells from pluripotent stem cells provides a versatile system for in vitro production of human neurons. PLoS One 7, e29597 (2012).
50. S. N. Djakovic, L. A. Schwarz, B. Barylko, G. N. DeMartino, G. N. Patrick, Regulation of the proteasome by neuronal activity and calcium/calmodulin-dependent protein kinase II. J. Biol. Chem. 284, 26655-26665 (2009).

### EXAMPLE 2

### T-Type Calcium Channel Inhibition—A Novel Therapeutic Target for Amyloid Diseases in the Brain

Objective

Pimozide is not specific for T-type calcium channels and is known and used for its action on dopaminergic receptor subtypes, serotoninergic receptors and T- and L-type calcium receptors. Penfluridol is also primarily known for its actions on dopaminergic receptor subtypes as well as actions on calcium channels that favour T-type over L-type.

The results of FIG. 4 exclude the possibility that the positive treatment effects are due to actions on dopaminergic or serotoninergic receptors because none of these experiments yielded any treatment effect. By using two T-type calcium channel-specific inhibitor (NNC55-0396 and ML-218) the inventors demonstrate that the target mechanism is the inhibition of T-type calcium channels (FIG. 5). Both T-type channel inhibitors replicated the results obtained with pimozide and penfluridol in mouse hippocampal slices.

Drosophila behavioural and histological experiments as well as the gamma oscillation rescue experiments in mouse hippocampal slices have been performed using an inhibitor of L-type calcium channels: verapamil. These behavioural tests show that inhibiting L-type calcium channels does not lead to a gain-of-function. Furthermore, the results show that inhibition of L-type calcium channels is unable to rescue gamma oscillations previously degraded by Aβ.

Results

L-Type Ca²⁺ Channel Inhibition Does Not Rescue Aβ-Induced Impairments in the Drosophila Climbing Assay

Wild type flies (welavGAL4^{c155} or UAS-Arc2E) and the progeny of the two control lines (ElavArc2E, overexpressing panneurally the human Aβ₁₋₄₂ with the Arctic mutation) flies were all kept at 23-25° C. divided in groups of 10-15 in 9 cm plastic vials with new food every 2-3 days. Both wild type and experimental flies were divided in groups with food containing the test compound (verapamil) from day 1 after hatching for all the length of the study (35 days) and groups with standard food containing just the vehicle in which verapamil was dissolved. Viable flies were counted daily to assess differences in longevity between wild type and experimental flies and between experimental not treated or treated with the compounds.

Climbing assays were performed on all flies every 5 days during the 35 days experimental period. Briefly, flies display a negative geotaxis response when given a mechanical stimulus. When tapped to the bottom of a vial, flies normally orient themselves rapidly and begin to climb to the top. By assaying the fly's ability to climb over a 10 cm line in an 18 cm vial in set time period (10 sec) we were able to compare broad nervous system function of reflex behaviours between flies with different genotypes and/or treated with different drugs. Climbing disability occurred in at least 50% of the ElavArc2E flies starting at 15 days after hatching and degenerating until day 35. Unlike in flies that were treated with pimozide no rescue in climbing ability could be observed in ElavArc2E flies treated with the L-type calcium channel inhibitor verapamil (5 μM; 15 days after hatching, ElavArc2E: 48.3±0.4% reached the top, ElavArc2E+verapamil: 49.7.2±0.5% reached the top, p=0.152; FIG. 11A). These behavioural tests show that inhibiting L-type calcium channels does not lead to a gain-of-function.

L-type Ca²⁺ Channel Inhibition Does Not Rescue Aβ-Plague Pathology in the Drosophila Immunohistochemistry Assay

At 35 days after hatching, flies were dissected and immunohistochemistry was performed on entire brains to study the number and size of amyloid plaques. All neurons were constitutively expressing GFP and the expression of the human-form of amyloid-β was panneural. Unlike in flies that were treated with pimozide no reduction of the number or the size of Aβ aggregates was evident in the mushroom bodies of the Drosophila's brains after treatment with verapamil (ElavArc2E: 0.52±0.04 μm², 0.017±0.003 aggregates/μm²; ElavArc2E+verapamil: 0.50±0.07 μm², 0.016±0.004 aggregates/pm²; p=0.090 for plaque size and p=0.104 for plaque frequency; FIG. 11B).

L-type Ca²⁺ Channel Inhibition is Not the Mechanism for the Rescue of the Aβ-Induced Impairment of Network Dynamics in Mice Brain Slices

Previously we have characterized the effects of acute Aβ on the hippocampal circuitry of WT mice. We reported that a physiological concentration of Aβ (50 nM) acutely administered degrades gamma oscillation in the hippocampal network (Kurudenkandy et al. 2014). After demonstrating the ability of T-type calcium channel inhibitors to rescue cognition-relevant gamma oscillations in mouse hippocampal slices after Aβ-induced degeneration we investigated whether the use of the L-type calcium channel inhibitor verapamil would have a similar effect or not.

Hippocampal slices were placed on a submerged recording chamber where gamma oscillations were induced by bath perfusion of 100 nM Kainate and allowed to stabilize for 20-30 minutes. Continuous recordings of LFP gamma oscillations were performed in 3 different conditions. All groups were initially activated with kainate. In a control group of slices oscillations were recorded for 40 minutes after their stabilization without any other compound addiction (control, n=5; see FIG. 5). All the other groups were perfused with Aβ 1 μM after the stabilization of the oscillations for a minimum of 20 minutes. For these experiments the concentration of Aβ applied was increased to 1 μM in order to overcome the method-dependent lower signal amplitude due to the submerged conditions (Kurudenkandy et al, 2014). After 20 minutes one group was left for additional 20 minutes in the same perfusion solution containing only Aβ (Aβ, n=5; see FIG. 5); for the third group the perfusion solution was enriched with 5 μM verapamil (Aβ+verapamil, n=5, FIG. 12) for additional 20 minutes to study the potential rescue effects.

Compared to control gamma oscillation power (4.73±0.75±10⁻⁹V², n=5, FIG. 5 and FIG. 12), hippocampal slices perfused with Aβ had a constant and fast decay of gamma oscillations, resulting after 40 minutes in a significant decrease of gamma oscillations power (0.23±0.05 of control, n=5, p=0.004, see FIG. 5 and FIG. 12). Gamma power measurement after application of verapamil showed no significant rescue of the impaired gamma power (Aβ+verapamil: 0.25±0.08 of control, n=5, p=0.004; FIG. 12). Our results show that inhibition of L-type calcium channels is unable to rescue gamma oscillations previously degraded by Aβ.

Materials and Methods

Drosophila

Genetics and Stocks

The binary UAS/Gal4 expression system was used throughout (Brand & Perrimon, 1993). Control flies were of the genotype welav-GAL4^{c155}GFP or UAS-Arc2E. Expression of the transgenes was achieved using the UAS/GAL4 system: UAS flies were crossed with flies expressing Gal4 under the control of a neuronal promoter (elav^{c155}). Experimental flies were of the following genotype: elav-Gal4/UAS-Arc2E (the most prone-to-aggregation Aβ₁₋₄₂). Elav-GAL4 drives panneural expression, so the experimental files were expressing panneurally the Arctic mutation.

All control and experimental flies were maintained on standard flour/agar fly food (H₂O, pure D(+)Glucose (Panreac), Instant Yeast (Anchor), Agar (Pronadisa), commercial flour (Gallo), Propionic Acid (Merck)) or with Pimozide 5 μM added to the standard food. All flies were maintained at 23-25° C. with a 12 h:12 h light:dark cycle. Food vials were changed every 2-3 days. Drug treatment (Pimozide, 5 μM (in DMSO, final dilution in standard food)) started at day 1 after hatching and continued throughout the all study length (35 days).

Negative Geotaxis Climbing Assay

Approximately 10 flies were placed in an empty plastic vial. The vial was gently tapped to knock the flies to the bottom and the flies were recorded during their subsequent climbing to the top of the vial (negative geotaxis). The number of flies at the top or bottom of the vial (i.e. flies able to cross a 2 cm line vs. flies not able to reach the line and/or cross it) was scored after 10 seconds. 10 trials were performed for each condition and each time point. The data shown represent results from a cohort of flies tested every 10 days for 35 days.

Data are presented as average±SEM. Results were tested for significance using ANOVA with Bonferroni Post Test to compare all different groups.

Whole-Mount Immunohistochemistry and Confocal Microscopy

Flies at 35 days of all genotypes were dissected (proboscis were removed from decapitated heads) in PBS buffer. Whole brains were fixed in 4% PFA (in PBS, pH 7.4) for 45 minutes: PFA solution was changed every 10 minutes. Following fixation, brains were washed for 30 minutes in 300 μM Glycine to avoid background staining due to the use of PFA. Brains were then washed 3 times with ice cold PBS containing 0.2% Triton X-100 for a total of 30 minutes to permeabilize the membranes. Subsequently they were blocked in 3% normal goat serum (NGS) for 2 hours at room temperature with gentle shake. Fly brains were then incubated overnight at 4° C. with gentle shake in mouse anti Aβ (6E10, BioLegend) diluted 1:800 in blocking buffer. After four further washes of 15 minutes at room temperature in PBST with gentle shake, brains were incubated in goat anti-mouse IgG Alexa 647 (Invitrogen). After 4 washes (15 minutes each) in PBST and 4 washes in PBS at room temperature with gentle shake, the brains were mounted in Vectashield (Vectorlabs) anti-fade mounting medium.

Confocal serial scanning images were acquired at 1 μm intervals with a Leica Confocal Microscope TCS SP5 II (Mannheim, Germany). Images were processed with using ImageJ (NIH): number of aggregates and respective sizes were measured and statistic was performed with GraphPad Prism (GraphPad Software, USA).

Mice

Experiments were performed in accordance with the ethical permit granted by Norra Stockholms Djurförsöksetiska Nämnd to André Fisahn (N45/13). C57BL/6 male mice (postnatal days 14-30, supplied from Charles River Laboratories, Germany) were used in this study. Animals were deeply anesthetized using isofluorane before being sacrificed by decapitation.

Drugs and Chemicals

All chemical compounds used in intracellular and extracellular solutions were obtained from Sigma-Aldrich Sweden AB (Stockholm, Sweden). Recombinant Aβ₁₋₄₂ was used in this study. Expression and purification of Aβ was previously reported (Kurudenkandy et al., 2014). Briefly, Met-A62 ₁₋₄₂ was expressed in Escherichia coli BL21 from synthetic genes and purified in batch format using ion exchange and passed through a 30000 Da Vivas-pin concentrator filter (Sartorius Stedim Biotech GmbH) to remove large aggregates. Purified peptide was concentrated to 50-100 μM, aliquoted in low-bind Eppendorf tubes (Axygene) and stored at −20° C. until use. Before use Aβ was thawed on ice and briefly sonicated 10 min before application.

Hippocampal Slice Preparation

The brain was dissected out and placed in ice-cold artificial cerebrospinal fluid (ACSF) modified for dissection containing (in mM) 80 NaCl, 24 NaHCO₃, 25 glucose, 1.25 NaH₂PO₄, 1 ascorbic acid, 3 Na pyruvate, 2.5 KCl, 4 MgCl₂, 0.5 CaCl2, 75 sucrose and bubbled with carbogen (95% O₂ and 5% CO₂). Horizontal sections (350 μm thick) of the ventral hippocampi of both hemispheres were prepared with a Leica VT1200S vibratome (Leica Microsystems). Immediately after cutting, slices were transferred into a humidified interface holding chamber containing standard ACSF (in mM): 124 NaCl, 30 NaHCO₃, 10 glucose, 1.25 NaH₂PO₄, 3.5 KCl, 1.5 MgCl₂, 1.5 CaCl₂, continuously supplied with humidified carbogen. The chamber was held at 34° C. during slicing and subsequently allowed to cool down to room temperature (˜22° C.) for a minimum of 1 hour.

Electrophysiology

Recordings were carried out in hippocampal area CA3 with borosilicate glass microelectrodes, pulled to a resistance of 3-7 MΩ. Local field potential (LFP) were recorded using microelectrodes filled with ACSF placed in CA3 stratum pyramidale. LFP oscillations were elicited by applying kainic acid (100 nM) to the extracellular bath. Interface chamber LFP recordings were performed with a 4-channel M102 amplifier (University of Cologne, Germany). Submerged chamber LFP recordings and patch-clamp recordings were performed using a Multiclamp 700B (Molecular Devices, CA, USA). In order to maintain stable LFP oscillations all recordings were performed at 34° C. with a perfusion rate of 3-5 ml per minute of aerated ACSF containing 100 nM Kainate. The oscillations were allowed to stabilize for at least 20 minutes before any recordings were performed.

The signals were sampled at 10 kHz, conditioned using a Hum Bug 50 Hz noise eliminator (Quest Scientific, North Vancouver, BC, Canada), software low-pass filtered at 1 kHz, digitized and stored using a Digidata 1440A and pCLAMP 10.4 software (Molecular Devices, CA, USA).

Data Analysis

Power spectra density plots (from 60 s long LFP recordings) were calculated in averaged Fourier-segments of 8192 points using Axograph X (Kagi, Berkeley, Calif., USA). Gamma oscillation power was calculated by integrating the power spectral density between 20 and 80 Hz.

Statistical Analysis

All statistical analysis was performed using GraphPad Prism. Results are reported as mean±SEM. Tests for statistical significance were performed on absolute values in all experiments using Wilcoxon matched-pairs test for paired data and Mann-Whitney U test for unpaired data. Significance levels were set as * p<0.05; ** p<0.01; *** p<0.001.

Embodiments of the Invention

The invention will now be described by the following numbered embodiments.
1. A calcium channel inhibitor for use in treating and/or preventing an amyloid disease of the nervous system in an individual.
2. Use of a calcium channel inhibitor in the manufacture of a medicament for treating and/or preventing an amyloid disease of the nervous system in an individual.
3. A method for treating and/or preventing an amyloid disease of the nervous system in an individual.
4. A calcium channel inhibitor for use according to Embodiment 1, or a use according to Embodiment 2, or a method according to Embodiment 3, wherein the amyloid disease of the nervous system is characterised by protein aggregation and/or protein misfolding.
5. A calcium channel inhibitor for use, or a use, or a method, according to Embodiment 4 wherein protein aggregation and/or protein misfolding causes the formation of one or more amyloid body, aggregate and/or assembly.
6. A calcium channel inhibitor for use, or a use, or a method, according to any of Embodiments 1 to 5 wherein the amyloid disease of the nervous system is selected from the group comprising: Alzheimer's disease (AD), Parkinson's disease (PD), Huntington's disease (HD), amyotrophic lateral sclerosis (ALS), Lewy-body dementia (LBD), a spongiform encephalopathy (such as Creutzfeldt Jakob Disease).
7. A calcium channel inhibitor for use, or a use, or a method, according to any preceding embodiment wherein the calcium channel inhibitor decreases cellular calcium in one or more cell.
8. A calcium channel inhibitor for use, or a use, or a method, according to any preceding embodiment wherein the calcium channel inhibitor is capable of increasing proteolysis.
9. A calcium channel inhibitor for use, or a use, or a method, of any preceding embodiment wherein the calcium channel inhibitor is capable of activating the proteasome.
10. A calcium channel inhibitor for use, or a use, or a method, according to any preceding embodiment wherein the calcium channel inhibitor is capable of reducing protein aggregation and/or protein misfolding.
11. A calcium channel inhibitor for use, or a use, or a method, according to any preceding embodiment wherein the calcium channel inhibitor is capable of reducing the number and/or size of amyloid aggregates in the nervous system of the individual.
12. A calcium channel inhibitor for use, or a use, or a method, according to Embodiment 11 wherein the amyloid aggregates are amyloid plaques, such as Aβ plaques.
13. A calcium channel inhibitor for use, or a use, or a method, according to any preceding embodiment wherein the calcium channel inhibitor is capable of preventing the loss of and/or restoring cognitive function.
14. A calcium channel inhibitor for use, or a use, or a method, according to Embodiment 13 wherein cognitive function is measured by determining neuronal oscillations in the brain of the individual.
15. A calcium channel inhibitor for use, or a use, or a method, according to Embodiment 14 wherein the neuronal oscillations are in the gamma-frequency and/or theta-frequency range.
16. A calcium channel inhibitor for use, or a use, or a method, according to any preceding embodiment wherein the calcium channel inhibitor is capable of fully or partially restoring action potential synchronization in the nervous system of the individual.
17. A calcium channel inhibitor, use or method of Embodiment 16 wherein action potential desynchronization is caused by protein aggregation and/or protein misfolding.
18. A calcium channel inhibitor for use, or a use, or a method, according to any preceding embodiment wherein the calcium channel inhibitor comprises a voltage gated calcium channel (VGCC) inhibitor.
19. A calcium channel inhibitor for use, or a use, or a method, according to Embodiment 18 wherein the VGCC inhibitor comprises an inhibitor of a T-type VGCC; or comprises an inhibitor of a T-type VGCC and an L-type VGCC.
20. A calcium channel inhibitor for use, or a use, or a method, according to any preceding embodiment wherein the calcium channel inhibitor is selected from the group comprising: a diphenylbutylpiperidine, a benzimidazole, 3-azabicyclo hexane, a quinazolin-2-one, a piperidine, a pyridine, a pyrazine, a piperazine (for example, a di-tert-butylphenyl piperazine or a piperazinylalkylpyrazole), an amino acid (for example, a (1-H-indol-3yl) ethylamine amino acid, a 3-(phenyl)acrylate ethylamine amino acid or an α,α spirocyclic amino acid), an N-piperidinyl acetamide, a 4-aminomethyl-piperidine, a bicyclic pyrimidine (for example, a 1,4-bisaminomethyl-cyclohexyl or a 4-(aminomethyl)-cyclohexylamine), a dihydropyrimidine, a dihydropyrimidone, a sulfonamide derivative, a substituted thiazole, a spiroazetidine, a spiroazetidinone, an oxadiazole (for example, a 5-methyl-oxadiazole), a benzhydryl, a benzenesulfonamide, a 3,4-dihydroquinazoline, a 2,4-dioxo-tetrahydroquinazoline, a 4-oxo-2-thioxo-tetrahydroquinazoline, a 1,3-dioxoisoindole, a 3-oxo-isoindoline, a morpholin-2-one, a 2-guanidino-thiazole, a 2-imino-1,3-thiazoline; a pyrrolidine (and open chain analogues thereof), Ethosuximide, Trimethadione, Zonisamide, Amlodipine, Aranidipine, Azelnidipine, Barnidipine, Benidipine, Efonidipine, Mibefradil, Nicardipine, Nimodipine, Lomerizine, A1048400, KYS05044, ML218, NNC 55-0396, RQ-00311610, TTA-A2, TTA-P2, VH04, Z941/944, pimozide, penfluridol, NNC55-0396 and ML-218.
21. A pharmaceutical composition comprising a calcium channel inhibitor as defined in any one of Embodiments 1-20, and a pharmaceutically acceptable diluent, carrier or excipient.
22. A pharmaceutical composition according to Embodiment 21, which further comprises one or more therapeutic agent for treating an amyloid disease of the nervous system.
23. A pharmaceutical composition according to Embodiment 21 or 22, for use in treating an amyloid disease of the nervous system in an individual.
24. A kit comprising:
   (i) an inhibitor as defined in any one of Embodiments 1-20;
   (ii) a pharmaceutically acceptable diluent, carrier or excipient; and/or
   (ii) at least one additional therapeutic agent.
25. A method for identifying an agent for treating and/or preventing an amyloid disease of the nervous system in an individual, the method comprising the steps of:
   (i) providing a candidate calcium channel inhibitor to be tested; and
   (ii) testing the candidate inhibitor in a model of neurodegenerative disease.
26. The method of Embodiment 25 further comprising the step of testing whether the candidate calcium channel inhibitor is capable of increasing proteolysis and/or activating the proteasome.
27. A calcium channel inhibitor for use, or a use, or a method, substantially as described herein, with reference to the accompanying description, examples and drawings.
28. A pharmaceutical composition, or a kit, or a pharmaceutical composition for use, or a kit for use, substantially as described herein, with reference to the accompanying description, examples and drawings.

1. A calcium channel inhibitor for use in treating and/or preventing an amyloid disease of the nervous system in an individual, wherein the calcium channel inhibitor is capable of preventing the loss of and/or restoring cognitive function, and wherein the calcium channel inhibitor is an inhibitor of a T-type voltage gated calcium channel (VGCC).

2. Use of a calcium channel inhibitor in the manufacture of a medicament for treating and/or preventing an amyloid disease of the nervous system in an individual, wherein the calcium channel inhibitor is capable of preventing the loss of and/or restoring cognitive function, and wherein the calcium channel inhibitor is an inhibitor of a T-type voltage gated calcium channel (VGCC).

3. A method for treating and/or preventing an amyloid disease of the nervous system in an individual comprising administering a calcium channel inhibitor to an individual, wherein the calcium channel inhibitor is capable of preventing the loss of and/or restoring cognitive function, and wherein the calcium channel inhibitor is an inhibitor of a T-type voltage gated calcium channel (VGCC).

4. A calcium channel inhibitor for use according to claim 1, or a use according to claim 2, or a method according to claim 3, wherein the amyloid disease of the nervous system is characterised by protein aggregation and/or protein misfolding.

5. A calcium channel inhibitor for use, or a use, or a method, according to claim 4 wherein protein aggregation and/or protein misfolding causes the formation of one or more amyloid body, aggregate and/or assembly.

6. A calcium channel inhibitor for use, or a use, or a method, according to any of claims 1 to 5 wherein the amyloid disease of the nervous system is selected from the group comprising: Alzheimer's disease (AD), Parkinson's disease (PD), Huntington's disease (HD), amyotrophic lateral sclerosis (ALS), Lewy-body dementia (LBS), a spongiform encephalopathy (such as Creutzfeldt Jakob Disease).

7. A calcium channel inhibitor for use, or a use, or a method, according to any preceding claim wherein the calcium channel inhibitor decreases cellular calcium in one or more cell.

8. A calcium channel inhibitor for use, or a use, or a method, according to any preceding claim wherein the calcium channel inhibitor is capable of increasing proteolysis.

9. A calcium channel inhibitor for use, or a use, or a method, of any preceding claim wherein the calcium channel inhibitor is capable of activating the proteasome.

10. A calcium channel inhibitor for use, or a use, or a method, according to any preceding claim wherein the calcium channel inhibitor is capable of reducing protein aggregation and/or protein misfolding.

11. A calcium channel inhibitor for use, or a use, or a method, according to any preceding claim wherein the calcium channel inhibitor is capable of reducing the number and/or size of amyloid aggregates in the nervous system of the individual.

12. A calcium channel inhibitor for use, or a use, or a method, according to claim 11 wherein the amyloid aggregates are amyloid plaques, such as Aβ plaques.

13. A calcium channel inhibitor for use, or a use, or a method, according to claim 12 wherein cognitive function is measured by determining neuronal oscillations in the brain of the individual.

14. A calcium channel inhibitor for use, or a use, or a method, according to claim 13 wherein the neuronal oscillations are in the gamma-frequency and/or theta-frequency range.

15. A calcium channel inhibitor for use, or a use, or a method, according to any preceding claim wherein the calcium channel inhibitor is capable of fully or partially restoring action potential synchronization in the nervous system of the individual.

16. A calcium channel inhibitor for use, or a use or a method of claim 15 wherein action potential desynchronization is caused by protein aggregation and/or protein misfolding.

17. A calcium channel inhibitor for use, or a use, or a method, according to any preceding claim wherein the calcium channel inhibitor is not pimozide, niguldipine, nicardipine, amiodarone and/or loperamide.

18. A calcium channel inhibitor for use, or a use, or a method, according to any of claims 1-16 wherein the calcium channel inhibitor is selected from the group comprising: a diphenylbutylpiperidine, a benzimidazole, 3-azabicyclo hexane, a quinazolin-2-one, a piperidine, a pyridine, a pyrazine, a piperazine (for example, a di-tert-butylphenyl piperazine or a piperazinylalkylpyrazole), an amino acid (for example, a (1-H-indol-3y1) ethylamine amino acid, a 3-(phenyl)acrylate ethylamine amino acid or an a,a spirocyclic amino acid), an N-piperidinyl acetamide, a 4-aminomethyl-piperidine, a bicyclic pyrimidine (for example, a 1,4-bisaminomethyl-cyclohexyl or a 4-(aminomethyl)-cyclohexylamine), a dihydropyrimidine, a dihydropyrimidone, a sulfonamide derivative, a substituted thiazole, a spiroazetidine, a spiroazetidinone, an oxadiazole (for example, a 5-methyl-oxadiazole), a benzhydryl, a benzenesulfonamide, a 3,4-dihydroquinazoline, a 2,4-dioxo-tetrahydroquinazoline, a 4-oxo-2-thioxo-tetrahydroquinazoline, a 1,3-dioxoisoindole, a 3-oxo-isoindoline, a morpholin-2-one, a 2-guanidine-thiazole, a 2-imino-1,3-thiazoline; a pyrrolidine (and open chain analogues thereof), Ethosuximide, Trimethadione, Zonisamide, Amlodipine, Aranidipine, Azelnidipine, Barnidipine, Benidipine, Efonidipine, Mibefradil, Nicardipine, Nimodipine, Lomerizine, A1048400, KYS05044, ML218, NNC 55-0396, RQ-00311610, TTA-A2, TTA-P2, VH04, Z941/944, pimozide, penfluridol, NNC55-0396 and ML-218.

19. A pharmaceutical composition comprising a calcium channel inhibitor as defined in any one of claims 1-18, and a pharmaceutically acceptable diluent, carrier or excipient.

20. A pharmaceutical composition according to claim 19, which further comprises one or more therapeutic agent for treating an amyloid disease of the nervous system.

21. A pharmaceutical composition according to claim 19 or 20, for use in treating an amyloid disease of the nervous system in an individual.

22. A kit comprising:
(i) an inhibitor as defined in any one of claims 1-18;
(ii) a pharmaceutically acceptable diluent, carrier or excipient; and/or
(ii) at least one additional therapeutic agent.

23. A method for identifying an agent for treating and/or preventing an amyloid disease of the nervous system in an individual, the method comprising the steps of:
(i) providing a candidate calcium channel inhibitor to be tested; and
(ii) testing the candidate inhibitor in a model of neurodegenerative disease.

24. The method of claim 23 further comprising the step of testing whether the candidate calcium channel inhibitor is capable of increasing proteolysis and/or activating the proteasome.

25. A calcium channel inhibitor for use, or a use, or a method, substantially as described herein, with reference to the accompanying description, examples and drawings.

26. A pharmaceutical composition, or a kit, or a pharmaceutical composition for use, or a kit for use, substantially as described herein, with reference to the accompanying description, examples and drawings.
